(19) United States
(12) Patent Application Publication
(10) Pub. No.: US 2019/0292189 A1
(43) Pub. Date: Sep. 26, 2019

(54) LIM KINASE INHIBITORS

(71) Applicant: VIRONGY, LLC, Manassas, VA (US)

(72) Inventors: Yangbo FENG, Manassas, VA (US); Yuntao WU, Manassas, VA (US)

(21) Appl. No.: 16/302,635

(22) PCT Filed: May 17, 2017
(86) PCT No.: PCT/US2017/033144

§ 371 Date: Nov. 17, 2018

Related U.S. Application Data
(60) Provisional application No. 62/338,040, filed on May 18, 2016.

(51) Int. Cl. C07D 487/04

(52) U.S. Cl. CPC C07D 487/04

(57) ABSTRACT

A dynamic actin cytoskeleton is necessary for viral entry, intracellular migration, and virion release. For the human immunodeficiency virus (HIV), during viral entry, the virus triggers early actin activity through hijacking chemokine coreceptor signaling, which activates a viral dependency host factor cofilin and its kinase, the LIM domain kinase (LIMK). Although knockdown of human LIMK1 with siRNA inhibits HIV infection, no specific small molecule inhibitor of LIMK is available. Here we describe the design and development of novel classes of small molecule inhibitors of human LIMK, based on different molecular scaffolds, for inhibiting infection by HIV, Ebola, and other viruses. Compounds of the invention can also be used for treatment of sexually transmitted diseases such as Herpes and Chlamydia.

### CROSS REFERENCE

This application claims benefit of U.S. Provisional Application No. 62/338,040, filed May 18, 2016, the content of which application is incorporated herein by reference in its entirety.

### STATEMENT OF GOVERNMENT SUPPORT

This invention was made with government support under EY021799 awarded by the National Institutes of Health. The government has certain rights in the invention.

### BACKGROUND

LIM-kinase (Limk) is a serine-threonine protein kinase. Two isoforms were identified as LIM kinase 1 (LIMK1, Limk1) and LIM kinase 2 (LIMK2, Limk2)¹⁻⁴ Limk1 and Limk2 are highly homologous and share 50% overall identity. Both isoforms consist of two amino-terminal LIM domains, adjacent PDZ and proline/serine-rich regions, followed by a carboxyl-terminal protein kinase domain.⁵ Limk1 was found to be expressed widely in embryonic and adult tissues, with notably high expression in the brain, kidney, lung, stomach and testis.⁶ Limk2 was found to be expressed in almost all embryonic and adult tissues examined with the exceptions of glial cell, the testis, and kidney glomeruli.⁷ Upon activation by upstream signals, Limk phosphorylates its substrate cofilin at the Ser-3 residue, thereby inactivating it, and leading to dynamic regulation of actin cytoskeleton.⁸⁻¹² Accumulated evidences suggest that Limk activity is associated with a variety of diseases including Williams Syndrome,¹³ Alzheimer Disease (AD),^{14, 15} psoriatic epidermal lesions,¹⁶ primary pulmonary hypertension (PPH),^{17, 18} intracranial aneurysms (IA),¹⁹ ocular hypertension/glaucoma,²⁰ HIV and other viral infections,²¹⁻²⁴ and cancers and cancer cell migration/invasion.²⁵⁻³¹

The LIMK1 gene is located in a region on human chromosome 7 (7q11.23) and is haplodeleted, along with 24 other genes, in people with Williams Syndrome (WS), a rare neurodevelopment genetic disorder associated with mild mental retardation and cardiovascular problem. The deletion of elastin (ELN) has been explicitly linked to most of the cardiovascular defects in WS. Though, the neurodevelopmental genotype-phenotype correlation is still uncertain, and may be related to the hemizygosity of WBSCR11, CYLN2, GTF2I, NCF1, and perhaps LIMK1. Specifically, LIMK1 knockout in mouse has been linked to alterations of hippocampal spine morphological and spatial learning. Nevertheless, LIMK1-null mice and children with human WS do not have the severe multiple developmental disorders normally seen in other developmental genetic diseases, suggesting that blocking LIMK1 is not fatal, and short-term or localized LIMK inhibition is likely tolerable in adults. Therefore, LIMK is considered a valuable target for treating various human diseases such as metastatic cancer, Alzheimer's disease, and drug addiction. For inhibiting HIV infection, LIMK1 can be stably knockdown (80-90%) by shRNA in human CD4 T cells, which renders T cells resistant to HIV infection. However, for anti-viral drug development, only a few small molecules have been shown to non-specifically modulate LIMK activity, and none has demonstrated a desirable high selectivity.

Recent molecular biology studies reported that Limk1 was over-expressed in cancerous prostate cells and tissues,²⁶ reduced expression of Limk1 retarded PC3ASL cells' proliferation by arresting cells at G2/M phase,²⁶ altered expression of Limk1 changed cell morphology and organization of actin cytoskeleton in PC3 cells,²⁶ increased expression of Limk1 was associated with accumulation of chromosomal abnormalities and development of cell cycle defects in cells that naturally express lower concentrations of Limk1,²⁷ reduced expression of Limk1 abolished the invasive behavior of prostate cancer cells,²⁷ and expression of Limk1 is higher in prostate tumors with higher Gleason Scores and incidence of metastasis.²⁷ All these observations suggest the possibility of up-regulated Limk1 as a cellular oncogene, and inhibition of Limk1 activity in cancerous prostate cells and tissues could lead to reduction of phosphorylated cofilin and decrease of the cells' motility and thus the invasiveness of tumor cells and their evolution to metastasis. Therefore, small molecule inhibitors of Limk1 could be potential therapeutic agents for prostate cancers. Recent studies also suggest that use of Limk inhibitors may provide a novel way to target the invasive machinery in GBM (glioblastoma multiforme).³²⁻³⁴

HIV-1 binding and entry into host cells are strongly impaired by the inhibition of actin polymerization.^{24, 35} Wu et al. demonstrated that HIV-mediated Limk activation is through gp120-triggered transient activation of the Rac-PAK-Limk pathway, and that knockdown of Limk through siRNA decreased filamentous actin, increased CXCR4 trafficking, and diminished viral DNA synthesis.²³ Wen et al. showed that LIM kinases modulate retrovirus particle release and cell-cell transmission events.²⁴ This research suggests that HIV hijacks Limk to control the cortical actin dynamics for the onset of viral infection of CD4 T cells. Therefore, Limk inhibitors are supposed to have high potentials as therapeutics in anti-HIV infection applications.²³

To the best of our knowledge, few small molecule Limk inhibitors have been reported in the literature.²⁸ Bristol-Myers Squibb pharmaceuticals (BMS) disclosed potent Limk1 inhibitors based on an aminothiazole scaffold.^{36, 37} Tel-Aviv University recently published an oxazole based Limk1/2 inhibitor (T56-Limki) from computer-aided drug design, which was found to be effective against cancer metastasis for treatment of neurofibromatosis.³⁴ A group of scientists from Australia reported 4-aminobenzothieno[3,2-d] pyrimidine based Limk1 inhibitors from high-through-put screen (HTS) showing activity in the micromolar range.^{38, 39} Recently, a Japanese group also reported a Limk inhibitor (Damnacanthal or Dam, natural product based) from HTS campaigns, and this compound (Dam) has a Limk1 inhibition IC₅₀ of ˜800 nM.³¹ Lexicon pharmaceuticals revealed a class of Limk inhibitors based on a piperidine urea or guanidine scaffold for the treatment of ocular hypertension and associated glaucoma.²⁰ More recently, the same group of Lexicon scientists reported a novel class of Type-III binding Limk2 inhibitors that are based on a sulfonamide scaffold.⁴⁰

### SUMMARY

The present invention is directed, in various embodiments, to a method of modulating a LIM kinase (LIMK), comprising contacting the LIM kinase with an effective amount or concentration of a compound of formula (I)

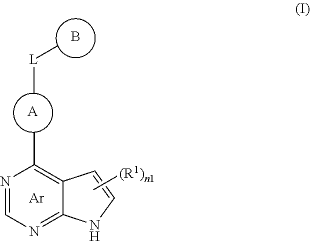

wherein

R¹ is halo, cyano (C₁-C₆)alkoxy, (C₁-C₆)alkyl, (C₂-C₆)alkenyl, or (C₂-C₆)alkynyl, wherein any alkyl, alkenyl, or alkynyl can be unsubstituted or substituted; wherein any one or two methylene groups thereof can be substituted with any of an independently selected NR′, S, O, C(═S), C(═O), OC(═O), OC(═O)O, OC(═O)NR′, C(═O)C(═O), SO₂NR′, S(O), S(O)₂, or C(═O)NR′NR′, wherein each independently selected R′ is H or (C₁-C₆)alkyl or (C₃-C₇)cycloalkyl;

n1=0, 1, or 2;

ring A is a 6-membered saturated ring comprising one or two nitrogen atoms, wherein the nitrogen atoms are disposed at the positions of ring A bonded to linker L, or to ring B when L is a direct bond, and to the ring system Ar; or ring A is a 5- or 6-membered aryl ring, a 5- or 6-membered heteroaryl ring, or a fused 6:5 heteroaryl ring system; ring A is substituted with n2 R² groups, wherein R² is halo, halo(C₁-C₆)alkyl, cyano, (C₁-C₆)alkoxy, (C₁-C₆)alkyl, (C₂-C₆)alkenyl, or (C₂-C₆)alkynyl, wherein any alkyl, alkenyl, or alkynyl can be unsubstituted or substituted; wherein any one or two methylene groups thereof can be substituted with any of an independently selected NR′, S, O, C(═S), C(═O), OC(═O), OC(═O)O, OC(═O)NR′, C(═O)C(═O), SO₂NR′, S(O), S(O)₂, or C(═O)NR′NR′, wherein each independently selected R′ is H or (C₁-C₆)alkyl or (C₃-C₇)cycloalkyl;

n2=0, 1, or 2;

L is a direct bond between ring A and ring B, or L is N(R³)C(═O)N(R³), wherein each R³ is independently H or (C₁-C₆)alkyl, wherein the R³ alkyl can be substituted with hydroxyl, (C₁-C₆)alkoxyl, amino, mono- or di-(C₁-C₆)alkylamino, or a 4- to 7-membered heterocyclyl ring;

ring B is a 5- or 6-membered aryl ring, a 5-membered or a 6-membered heteroaryl ring, or a fused 6:5 heteroaryl ring system; ring B is substituted with n4 R⁴ groups, wherein R⁴ is halo, cyano, (C₁-C₆)alkyl, (C₂-C₆)alkenyl, (C₂-C₆)alkynyl, (C₁-C₆)alkoxy, (C₁-C₆)alkoxycarbonyl, (C₃-C₇)cycloalkoxycarbonyl, R′NHC(═O), or R′₂NC(═O), wherein any alkyl, alkenyl, or alkynyl can be unsubstituted or substituted; wherein any one or two methylene groups thereof can be substituted with any of an independently selected NR′, S, O, C(═S), C(═O), OC(═O), OC(═O)O, OC(═O)NR′, C(═O)C(═O), SO₂NR′, S(O), S(O)₂, or C(═O)NR′NR′, wherein each independently selected R′ is H or (C₁-C₆)alkyl or (C₃-C₇)cycloalkyl;

or a pharmaceutically acceptable salt thereof.

In various embodiments, a method of the invention can use a compound of formula (IA)

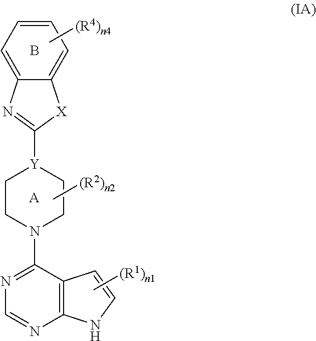

wherein

Y is CR³ or N;

X is NR³, CH₂, CHR¹, O, or S;

wherein R¹, n1, R², n2, R³, R⁴, and n4, are as defined herein.

In various embodiments, a method of the invention can use a compound of formula (IB)

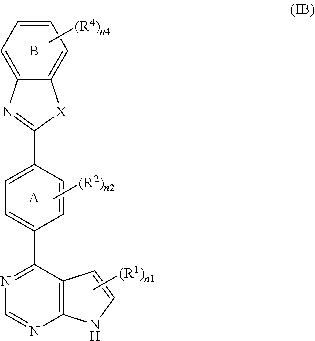

X is NR³, CH₂, CHR¹, O, or S;

wherein R¹, n1, R², n2, R³, R⁴, and n4, are as defined herein.

In various embodiments, a method of the invention can use any of the effective LIM kinase inhibitors disclosed and claimed herein.

The invention further can provide, in various embodiments, a method of treating a condition in a patient, wherein modulating a LIM kinase is medically indicated, comprising administering to the patient an effective amount of a compound of formula (I). For instance, the condition can be a viral infection, a metastatic condition, Alzheimer's disease, glaucoma, psoriasis, or a drug addiction. More specifically, the viral infection can be a human immunodeficiency viral (HIV) infection, an Ebola viral (EBOV) infection, a Rift Valley Fever viral (RVFV) infection, a Venezuelan equine encephalitis viral (VEEV) infection, or a Herpes Simplex 1 viral (HSV-1) infection.

The invention can provide, in various embodiments, bioactive compounds and formulations thereof for the treatment of sexually transmitted diseases, such as those caused by the Herpes virus, or bacteria such as Chlamydia.

In various embodiments, the invention provides a compound of formula (I), of formula (IA), of formula (IB), or any of the effective LIM kinase modulatory compounds disclosed and claimed herein.

### BRIEF DESCRIPTION OF THE FIGURES

FIG. 1. Transition from ROCK inhibition to LIMK inhibition for the phenyl urea based scaffold of kinase inhibitors.

FIG. 2. Screening LIMK inhibitors for anti-HIV activity. a, An HIV Rev-dependent indicator T cell, Rev-CEM-GFP-Luc, was pretreated with LIMK inhibitors or DMSO for 1 hour, and then infected with HIV-1(NL4-3) for 3 hours. Cells were washed to remove the virus and the inhibitors, and incubated for 48 hours. HIV-dependent GFP expression was measured by flow cytometry. Propidum iodide (PI) was added during flow cytometry to simultaneously measure cell viability. Only viable cells were used for GFP quantification. The relative infection rate in drug treated versus DMSO treated cells (100%) were plotted using the relative percent of GFP+ cells. b, The IC50 of R10015 was measured by luciferase assay following drug treatment and infection of Rev-CEM-GFP-Luc (red triangle). For comparison, cells were also treated with 100 μM of R10015, and infected with VSV-G pseudotype HIV (black dot). c, The cytotoxicity of R10015 was simultaneously measured by propidium iodide (PI) stained and flow cytometry. d, flow cytometry results demonstrating that R10015 inhibits HIV-1(NL4-3), but not HIV-1(VSVG), Rev-CEM-GFP-Luc was used.

FIG. 3. Screening LIMK inhibitors for anti-HIV activity. An HIV Rev-dependent indicator T cell, Rev-CEM-GFP-Luc, was pretreated with LIMK inhibitors or DMSO for 1 hour, and then infected with HIV-1(NL4-3) for 3 hours. Cells were washed to remove the virus and the inhibitors, and incubated for 48 hours. HIV-dependent GFP expression was measured by flow cytometry. Propidum iodide (PI) was added during flow cytometry to simultaneously measure cell viability. Only viable cells (gate R1) were used for GFP quantification. The percents of GFP+ cells are shown.

FIG. 4. Substitutions on the urea NH attached to the central phenyl group were not tolerated.

FIG. 5. Docking of 18b to the crystal structure of Limk1 (PDB ID 3S95). A) Schematic view showing key interactions. B) Surface view showing the binding pockets.

FIG. 6. A: Western blot analysis of p-cofilin in PC-3 prostate cancer cell lines stimulated by HGF treated with 18b. Similar cell potency was also observed for compound 18f in PC-3 cells. B: Western blot analysis of p-cofilin in CEM-SS T cell lines for 18p, 18r, and 18x.

FIG. 7. Effect of Limk inhibitors on invasion of PC-3 cell. Comparison of cell invasion (left panel) and phase contrast images (right panel) by treatment of 18b (1 μM) or 18f (1 μM) for 48 hours in PC-3 cells. The results are shown as mean±SD of one representative experiment (from three independent experiments) performed in triplicate. Statically significant differences are indicated (*) p<0.05.

FIG. 8. Effect of Limk1 inhibitors on migration of PC-3 cells. Comparison of the average (%) of wound closure (left panel) and phase contrast images (right panel) by treatment with indicated concentration of 18b (A) or 18f (B) for 24 hours in PC-3 cells. The results are shown as mean±SD of one representative experiment (from three independent experiments) performed in triplicate. Statistically significant differences are indicated (NS) no significance, and (*) p<0.01. Scale bar: 20 μm.

FIG. 9. IOP lowering effect of 18w on rat eyes. Topical dosing at 50 μg. Data were averaged from 6 determinations (based on 6 rats).

FIG. 10. Chemical and Biochemical characterization of R10015. a, Chemical structure of R10015 and its docking into the crystal structure of LIMK1 (PDB ID 3S95, Chain A). The binding motif of R10015 shows that it is a typical Type-I ATP-competitive kinase inhibitor. b, The 10-dose inhibition curve of R10015 against the recombinant LIMK1 enzyme (IC50=38±5 nM). c, R10015 blocks cofilin serine 3 phosphorylation in human T cells. CEM-SS T cells treated with R10015 (100 μM) for a time course, and the phosphorylation of cofilin, LIMK, and PAK1/2 were measured by Western Blot. Levels of each protein or GAPDH were also measured for loading control. d, R10015 inhibits Jurkat T cell chemotaxis in responding to SDF-1. e, R10015. f, R10015 inhibits chemotactic actin activity. Resting CD4 T cells were treated with R10015, and then exposed to SDF-1 for a time course. Actin polymerization were measured by FITC-phalloidin staining. g, the relative intensity of F-actin staining was also plotted.

FIG. 11. R10015 inhibits HIV-1 DNA synthesis, nuclear migration, and virion release. a, Effects of R10015 on surface CD4 and CXCR4 expression. CEM-SS T cells were treated with R10015 (100 μM), and then stained for surface CD4 or CXCR4. b, R10015 did not inhibit viral entry. CEM-SS T cells were treated with R10015, and then infected with BlaM-Vpr tagged HIV-1(NL4-3) or HIV-1(VSV-G) to measure viral entry. c. R10015 inhibits viral DNA synthesis. CEM-SS T cells were treated with R10015 for 1 hour, and then infected with a single-cycle HIV-1(Env) for 2 hours in the presence of R10015. Following infection, cells were washed to remove HIV-1 and R10015. Viral DNA synthesis was measured by real-time PCR. d. R10015 inhibits 2-LTR circle DNA formation. Cells were similar treated with R10015 and infected. 2-TLR circles were quantified by real-time PCR. e. R10015 inhibits virion release. Cells were infected with HIV-1(Env) for 2 hours, washed, incubated for 12 hours, and then treated with R10015. Virion release was quantified by measuring p24 in the supernatant.

FIG. 12. R10015 inhibits HIV-1, EBOV, VEEV, RVFV, and HSV-1 infection. a, R10015 inhibits HIV latent infection of resting CD4+ T cells. Cells were treated with R10015 (100 μM) or DMSO for 1 hour, infected with HIV-1(NL4-3) for 2 hours, washed, cultured for 5 days in the absence of R10015, and then activated with CD3/CD28 beads. Viral p24 release was measured. b, CD25 and CD69 surface staining demonstrate that R10015 did not inhibit T cell activation. c, R10015 inhibits R5 HIV-1 latent infection of CD45RO+ memory CD4 T cells. Cells were similarly treated with R10015, infected with HIV-1(AD8), washed, incubated, and then activated. d, CD69 surface staining were performed for control on R10015 effects on T cell activation. e to g, R10015 inhibits EBOV infection. HFF-1 cells were treated with R10015 for 2 hours, infected with EBOV (Zaire) (MOI, 2.5) for 48 hours in the presence of R10015. Cells were fixed and stained for the EBOV GP protein with an Alexa 488-labelled antibody (g, green), or with Hoechst for the nuclei (g, blue). The relative GP protein staining was converted to percentage inhibition using the infected, drug-untreated cells as the control (a). Cell viability was calculated based on the number of nuclei per well in comparison with uninfected control (b). g is the confocal imaging of stained cells with infection and drug treatment. h, R10015 inhibits HSV-1 infection. Vero cells were pretreated with R10015 (100 μM) or DMSO for 2 hours, and then infected with serially diluted HSV-1 for 2 hours. Following infection, cells were washed, and cultured in the absence of R10015. Viral plaques were stained quantified with Giemsa stain. i, R10015 inhibits VEEV infection, Vero cells were treated with R10015 or DMSO, infected with a luciferase reporter virus, VEEV-Luc(TC-83), VEEV(TC-83), or VEEV(TrD) (MOI, 0.1), R10015 was added into the medium post infection. No drug-induced cytotoxicity was observed during the infection period (data not shown). Viral supernatants were collected 24 hours post infection, and analyzed with luciferase or plaque assay. j, R10015 inhibits RVFV infection. Vero cells were similarly treated with R10015, infected with RVFVLuc (MP12) (MOI, 0.1), and analyzed with luciferase assay.

### DETAILED DESCRIPTION

As used in the specification and the appended claims, the singular forms “a,” “an” and “the” include plural referents unless the context clearly dictates otherwise.

The term “about” as used herein, when referring to a numerical value or range, allows for a degree of variability in the value or range, for example, within 10%, or within 5% of a stated value or of a stated limit of a range.

All percent compositions are given as weight-percentages, unless otherwise stated.

As used herein, “individual” (as in the subject of the treatment) or “patient” means both mammals and non-mammals. Mammals include, for example, humans; non-human primates, e.g. apes and monkeys; and non-primates, e.g. dogs, cats, cattle, horses, sheep, and goats. Non-mammals include, for example, fish and birds.

The term “disease” or “disorder” or “malcondition” are used interchangeably, and are used to refer to diseases or conditions wherein a LIM kinase (LIMK) plays a role in the biochemical mechanisms involved in the disease or medical condition or symptom(s) thereof such that a therapeutically beneficial effect can be achieved by acting on LIMK, e.g. with an effective amount or concentration of a synthetic ligand of the invention. “Acting on” LIMK, or “modulating” LIMK, can include binding to LIMK and/or inhibiting the bioactivity of LIMK and/or allosterically regulating the bioactivity of LIMK in vivo.

The expression “effective amount”, when used to describe therapy to an individual suffering from a disorder, refers to the quantity or concentration of a compound of the invention that is effective to inhibit or otherwise act on LIMK in the individual's tissues wherein LIMK involved in the disorder, wherein such inhibition or other action occurs to an extent sufficient to produce a beneficial therapeutic effect.

“Treating” or “treatment” within the meaning herein refers to an alleviation of symptoms associated with a disorder or disease, or inhibition of further progression or worsening of those symptoms, or prevention or prophylaxis of the disease or disorder, or curing the disease or disorder. Similarly, as used herein, an “effective amount” or a “therapeutically effective amount” of a compound of the invention refers to an amount of the compound that alleviates, in whole or in part, symptoms associated with the disorder or condition, or halts or slows further progression or worsening of those symptoms, or prevents, or provides prophylaxis for, the disorder or condition. In particular, a “therapeutically effective amount” refers to an amount effective, at dosages and for periods of time necessary, to achieve the desired therapeutic result. A therapeutically effective amount is also one in which any toxic or detrimental effects of compounds of the invention are outweighed by the therapeutically beneficial effects.

Phrases such as “under conditions suitable to provide” or “under conditions sufficient to yield” or the like, in the context of methods of synthesis, as used herein refers to reaction conditions, such as time, temperature, solvent, reactant concentrations, and the like, that are within ordinary skill for an experimenter to vary, that provide a useful quantity or yield of a reaction product. It is not necessary that the desired reaction product be the only reaction product or that the starting materials be entirely consumed, provided the desired reaction product can be isolated or otherwise further used.

It is to be further understood that where descriptions of various embodiments use the term “comprising,” those skilled in the art would understand that in some specific instances, an embodiment can be alternatively described using language “consisting essentially of” or “consisting of.”

By “chemically feasible” is meant a bonding arrangement or a compound where the generally understood rules of organic structure are not violated; for example a structure within a definition of a claim that would contain in certain situations, e.g., a pentavalent carbon atom that would not exist in nature would be understood to not be within the claim. The structures disclosed herein, in all of their embodiments are intended to include only “chemically feasible” structures, and any recited structures that are not chemically feasible, for example in a structure shown with variable atoms or groups, are not intended to be disclosed or claimed herein.

When a substituent is specified to be an atom or atoms of specified identity, “or a bond”, a configuration is referred to when the substituent is “a bond” that the groups that are immediately adjacent to the specified substituent are directly connected to each other in a chemically feasible bonding configuration.

All single enantiomer, diastereomeric, and racemic forms of a structure are intended, unless a particular stereochemistry or isomeric form is specifically indicated. In several instances though an individual stereoisomer is described among specifically claimed compounds, the stereochemical designation does not imply that alternate isomeric forms are less preferred, undesired, or not claimed. Compounds used in the present invention can include enriched or resolved optical isomers at any or all asymmetric atoms as are apparent from the depictions, at any degree of enrichment. Both racemic and diastereomeric mixtures, as well as the individual optical isomers can be isolated or synthesized so as to be substantially free of their enantiomeric or diastereomeric partners, and these are all within the scope of the invention.

As used herein, the terms “stable compound” and “stable structure” are meant to indicate a compound that is sufficiently robust to survive isolation to a useful degree of purity from a reaction mixture, and formulation into an efficacious therapeutic agent. Only stable compounds are contemplated herein.

When a group is recited, wherein the group can be present in more than a single orientation within a structure resulting in more than single molecular structure, e.g., a carboxamide group C(═O)NR, it is understood that the group can be present in any possible orientation, e.g., X—C(═O)N(R)—Y or X—N(R)C(═O)—Y, unless the context clearly limits the orientation of the group within the molecular structure.

When a group, e.g., an “alkyl” group, is referred to without any limitation on the number of atoms in the group, it is understood that the claim is definite and limited with respect the size of the alkyl group, both by definition; i.e., the size (the number of carbon atoms) possessed by a group such as an alkyl group is a finite number, bounded by the understanding of the person of ordinary skill as to the size of the group as being reasonable for a molecular entity; and by functionality, i.e., the size of the group such as the alkyl group is bounded by the functional properties the group bestows on a molecule containing the group such as solubility in aqueous or organic liquid media. Therefore, a claim reciting an “alkyl” or other chemical group or moiety is definite and bounded, as the number of atoms in the group cannot be infinite and is limited by ordinary understanding.

It has become widely recognized by those skilled in the art that the incorporation of isotopic forms of an atom may impart useful properties. For example, a deuterium atom (²H) may be specifically introduced in place of a hydrogen atom which would otherwise represent the natural distribution of hydrogen isotopes, mostly ¹H. The use of one or more such isotopic substitutions may alter the properties of the resultant composition, including alterations in relevant properties in a treated animal, such as a longer half-life or duration of action of the composition. The isotope may also enable methods to detect the amount of the composition in affected tissue, such as by detection of radiation from isotopes such as ³H and ¹⁴C. Chemical methods for incorporating isotopes (examples including, but not limited to, ²H, 3H, ¹³C, ¹⁴C) are well-known in the art and the claims of this invention encompass such isotopic forms.

In general, “substituted” refers to an organic group as defined herein in which one or more bonds to a hydrogen atom contained therein are replaced by one or more bonds to a non-hydrogen atom such as, but not limited to, a halogen (e.g., F, Cl, Br, or I); an oxygen atom in groups such as hydroxyl groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxylamines, nitriles, nitro groups, nitroso groups, N-oxides, hydrazides, azides, and enamines; and other heteroatoms in various other groups. Non-limiting examples of substituents that can be bonded to a substituted carbon (or other) atom include F, Cl, Br, I, OR, CN, NO, NO₂, ONO₂, azido, CF₃, OCF₃, R, O (oxo), S (thiono), methylenedioxy, ethylenedioxy, N(R), SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, or C(═NOR)R wherein R can be hydrogen or a carbon-based moiety, and wherein the carbon-based moiety can itself be further substituted; for example, R can be hydrogen, alkyl, acyl, cycloalkyl, aryl, aralkyl, heterocyclyl, heteroaryl, or heteroarylalkyl, wherein any alkyl, acyl, cycloalkyl, aryl, aralkyl, heterocyclyl, heteroaryl, or heteroarylalkyl can be further independently mono- or multi-substituted with J, or with some or all of the above-listed functional groups, or with other functional groups; or wherein two R groups bonded to a nitrogen atom or to adjacent nitrogen atoms can together with the nitrogen atom or atoms form a heterocyclyl, which can be further mono- or independently multi-substituted with J, or with some or all of the above-listed functional groups, or with other functional groups.

In various embodiments, a substituent can be any of halo, (C1-C6)alkyl, (C1-C6)alkoxy, (C1-C6)haloalkyl, hydroxy(C1-C6)alkyl, alkoxy(C1-C6)alkyl, (C1-C6)alkanoyl, (C1-C6)alkanoyloxy, cyano, nitro, azido, R₂N, R₂NC(O), R₂NC(O)O, R₂NC(O)NR, (C1-C6)alkenyl, (C1-C6)alkynyl, (C6-C10)aryl, (C6-C10)aryloxy, (C6-C10)aroyl, (C6-C10)aryl(C1-C6)alkyl, (C6-C10)aryl(C1-C6)alkoxy, (C6-C10)aryloxy(C1-C6)alkyl, (C6-C10)aryloxy(C1-C6)alkoxy, (3- to 9-membered)heterocyclyl, (3- to 9-membered)heterocyclyl(C1-C6)alkyl, (3- to 9-membered)heterocyclyl(C1-C6)alkoxy, (5- to 10-membered)heteroaryl, (5- to 10-membered)heteroaryl(C1-C6)alkyl, (5- to 10-membered)heteroaryl(C1-C6)alkoxy, or (5- to 10-membered)heteroaroyl. For example, R independently at each occurrence can be H, (C1-C6)alkyl, or (C6-C10)aryl, wherein any alkyl or aryl group is substituted with 0-3 J.

In various embodiments, a substituent can be any of halo, (C1-C6)alkyl, (C1-C6)alkoxy, (C1-C6)haloalkyl, hydroxy(C1-C6)alkyl, alkoxy(C1-C6)alkyl, (C1-C6)alkanoyl, (C1-C6)alkanoyloxy, cyano, nitro, azido, R₂N, R₂NC(O), R₂NC(O)O, R₂NC(O)NR, (C1-C6)alkenyl, (C1-C6)alkynyl, (C6-C10)aryl, (C6-C10)aryloxy, (C6-C10)aroyl, (C6-C10)aryl(C1-C6)alkyl, (C6-C10)aryl(C1-C6)alkoxy, (C6-C10)aryloxy(C1-C6)alkyl, (C6-C10)aryloxy(C1-C6)alkoxy, (3- to 9-membered)heterocyclyl, (3- to 9-membered)heterocyclyl(C1-C6)alkyl, (3- to 9-membered)heterocyclyl(C1-C6)alkoxy, (5- to 10-membered)heteroaryl, (5- to 10-membered)heteroaryl(C1-C6)alkyl, (5- to 10-membered)heteroaryl(C1-C6)alkoxy, or (5- to 10-membered)heteroaroyl. For example, R independently at each occurrence can be H, (C1-C6)alkyl, or (C6-C10)aryl, wherein any alkyl or aryl group is substituted with 0-3 J.

When a substituent is monovalent, such as, for example, F or Cl, it is bonded to the atom it is substituting by a single bond. When a substituent is more than monovalent, such as O, which is divalent, it can be bonded to the atom it is substituting by more than one bond, i.e., a divalent substituent is bonded by a double bond; for example, a C substituted with O forms a carbonyl group, C═O, which can also be written as “CO”, “C(O)”, or “C(═O)”, wherein the C and the O are double bonded. When a carbon atom is substituted with a double-bonded oxygen (═O) group, the oxygen substituent is termed an “oxo” group. When a divalent substituent such as NR is double-bonded to a carbon atom, the resulting C(═NR) group is termed an “imino” group. When a divalent substituent such as S is double-bonded to a carbon atom, the results C(═S) group is termed a “thiocarbonyl” or “thiono” group.

Alternatively, a divalent substituent such as O or S can be connected by two single bonds to two different carbon atoms. For example, O, a divalent substituent, can be bonded to each of two adjacent carbon atoms to provide an epoxide group, or the O can form a bridging ether group, termed an “oxa” or “oxy” group, between adjacent or non-adjacent carbon atoms, for example bridging the 1,4-carbons of a cyclohexyl group to form a [2.2.1]-oxabicyclo system. Further, any substituent can be bonded to a carbon or other atom by a linker, such as (CH₂)ₙ or (CR₂)ₙ wherein n is 1, 2, 3, or more, and each R is independently selected.

Another divalent substituent is an alkylidene carbon, represented as C═ and signifying that the carbon atom so indicated, which also bears two additional groups, is double bonded to a third group. For example, (CH₃)₂C═ indicates an isopropylidene group bonded to another carbon or nitrogen atom.

C(O) and S(O)₂ groups can also be bound to one or two heteroatoms, such as nitrogen or oxygen, rather than to a carbon atom. For example, when a C(O) group is bound to one carbon and one nitrogen atom, the resulting group is called an “amide” or “carboxamide.” When a C(O) group is bound to two nitrogen atoms, the functional group is termed a “urea.” When a C(O) is bonded to one oxygen and one nitrogen atom, the resulting group is termed a “carbamate” or “urethane.” When a S(O)₂ group is bound to one carbon and one nitrogen atom, the resulting unit is termed a “sulfonamide.” When a S(O)₂ group is bound to two nitrogen atoms, the resulting unit is termed a “sulfamide.”

Substituted alkyl, alkenyl, alkynyl, cycloalkyl, and cycloalkenyl groups as well as other substituted groups also include groups in which one or more bonds to a hydrogen atom are replaced by one or more bonds, including double or triple bonds, to a carbon atom, or to a heteroatom such as, but not limited to, oxygen in carbonyl (oxo), carboxyl, ester, amide, imide, urethane, and urea groups; and nitrogen in imines, hydroxyimines, oximes, hydrazones, amidines, guanidines, and nitriles.

Substituted ring groups such as substituted cycloalkyl, aryl, heterocyclyl and heteroaryl groups also include rings and fused ring systems in which a bond to a hydrogen atom is replaced with a bond to a carbon atom, or to a substituent group as defined above. Therefore, substituted cycloalkyl, aryl, heterocyclyl and heteroaryl groups can also be substituted with alkyl, alkenyl, and alkynyl groups, or with the substituent groups listed above or other substituent groups know to persons of ordinary skill in the art.

By a “ring system” as the term is used herein is meant a moiety comprising one, two, three or more rings, which can be substituted with non-ring groups or with other ring systems, or both, which can be fully saturated, partially unsaturated, fully unsaturated, or aromatic, and when the ring system includes more than a single ring, the rings can be fused, bridging, or spirocyclic. Ring systems can be mono- or independently multi-substituted with substituents as are described above. By “spirocyclic” is meant the class of structures wherein two rings are fused at a single tetrahedral carbon atom, as is well known in the art.

As to any of the groups described herein, which contain one or more substituents, it is understood, of course, that such groups do not contain any substitution or substitution patterns which are sterically impractical and/or synthetically non-feasible. In addition, the compounds of this disclosed subject matter include all stereochemical isomers arising from the substitution of these compounds.

When a number of carbon atoms in a group, e.g., an alkyl, alkenyl, alkynyl, cycloalkyl, aryl, etc., is specified as a range, each individual integral number representing the number of carbon atoms is intended. For example, recitation of a (C₁-C₄)alkyl group indicates that the alkyl group can be any of methyl, ethyl, propyl, isopropyl, butyl, sec-butyl, isobutyl, or tert-butyl. It is understood that a specification of a number of carbon atoms must be an integer.

When a number of atoms in a ring is specified, e.g., a 3- to 9-membered cycloalkyl or heterocyclyl ring, the cycloalkyl or heterocyclyl ring can include any of 3, 4, 5, 6, 7, 8, or 9 atoms. A cycloalkyl ring is carbocyclic; a heterocyclyl ring can include atoms of any element in addition to carbon capable of forming two or more bonds, e.g., nitrogen, oxygen, sulfur, and the like. The number of atoms in a ring is understood to necessarily be an integer.

Alkyl groups include straight chain and branched carbon-based groups having from 1 to about 20 carbon atoms, and typically from 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the substituent groups listed above, for example, amino, hydroxy, cyano, carboxy, nitro, thio, alkoxy, and halogen groups. Exemplary alkyl groups include, but are not limited to, straight or branched hydrocarbons of 1-6, 1-4, or 1-3 carbon atoms, referred to herein as C₁₋₆alkyl, C₁₋₄alkyl, and C₁₋₃alkyl, respectively. Exemplary alkyl groups include, but are not limited to, methyl, ethyl, propyl, isopropyl, 2-methyl-1-butyl, 3-methyl-2-butyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, etc.

Cycloalkyl groups are groups containing one or more carbocyclic ring including, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined above.

Representative substituted cycloalkyl groups can be mono-substituted or substituted more than once, such as, but not limited to, 2,2-, 2,3-, 2,4- 2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group, i.e., a cycloalkyl including one or more carbon-carbon double bond.

The terms “carbocyclic,” “carbocyclyl,” and “carbocycle” denote a ring structure wherein the atoms of the ring are carbon, such as a cycloalkyl group or an aryl group. In some embodiments, the carbocycle has 3 to 8 ring members, whereas in other embodiments the number of ring carbon atoms is 4, 5, 6, or 7. Unless specifically indicated to the contrary, the carbocyclic ring can be substituted with as many as N−1 substituents wherein N is the size of the carbocyclic ring with, for example, alkyl, alkenyl, alkynyl, amino, aryl, hydroxy, cyano, carboxy, heteroaryl, heterocyclyl, nitro, thio, alkoxy, and halogen groups, or other groups as are listed above. A carbocyclyl ring can be a cycloalkyl ring, a cycloalkenyl ring, or an aryl ring. A carbocyclyl can be monocyclic or polycyclic, and if polycyclic each ring can be independently be a cycloalkyl ring, a cycloalkenyl ring, or an aryl ring.

(Cycloalkyl)alkyl groups, also denoted cycloalkylalkyl, are alkyl groups as defined above in which a hydrogen or carbon bond of the alkyl group is replaced with a bond to a cycloalkyl group as defined above.

Alkenyl groups include straight and branched chain and cyclic alkyl groups as defined above, except that at least one double bond exists between two carbon atoms. Thus, alkenyl groups have from 2 to about 20 carbon atoms, and typically from 2 to 12 carbons or, in some embodiments, from 2 to 8 carbon atoms. Examples include, but are not limited to vinyl, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others. Exemplary alkenyl groups include, but are not limited to, a straight or branched group of 2-6 or 3-4 carbon atoms, referred to herein as C₂₋₆alkenyl, and C₃₋₄alkenyl, respectively. Exemplary alkenyl groups include, but are not limited to, vinyl, allyl, butenyl, pentenyl, etc.

Cycloalkenyl groups include cycloalkyl groups having at least one double bond between 2 carbons. Thus for example, cycloalkenyl groups include but are not limited to cyclohexenyl, cyclopentenyl, and cyclohexadienyl groups. Cycloalkenyl groups can have from 3 to about 8-12 ring members, whereas in other embodiments the number of ring carbon atoms range from 3 to 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like, provided they include at least one double bond within a ring. Cycloalkenyl groups also include rings that are substituted with straight or branched chain alkyl groups as defined above.

(Cycloalkenyl)alkyl groups are alkyl groups as defined above in which a hydrogen or carbon bond of the alkyl group is replaced with a bond to a cycloalkenyl group as defined above.

Alkynyl groups include straight and branched chain alkyl groups, except that at least one triple bond exists between two carbon atoms. Thus, alkynyl groups have from 2 to about 20 carbon atoms, and typically from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

Aryl groups are cyclic aromatic hydrocarbons that do not contain heteroatoms in the ring. An aromatic compound, as is well-known in the art, is a multiply-unsaturated cyclic system that contains 4n+2 π electrons where n is an integer. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined above. Representative substituted aryl groups can be mono-substituted or substituted more than once, such as, but not limited to, 2-, 3-, 4-, 5-, or 6-substituted phenyl or 2-8 substituted naphthyl groups, which can be substituted with carbon or non-carbon groups such as those listed above.

Aralkyl, also termed arylalkyl, groups are alkyl groups as defined above in which a hydrogen or carbon bond of an alkyl group is replaced with a bond to an aryl group as defined above. Representative aralkyl groups include benzyl and phenylethyl groups and fused (cycloalkylaryl)alkyl groups such as 4-ethyl-indanyl. Aralkenyl group are alkenyl groups as defined above in which a hydrogen or carbon bond of an alkyl group is replaced with a bond to an aryl group as defined above.

Heterocyclyl groups or the term “heterocyclyl” includes aromatic and non-aromatic ring compounds containing 3 or more ring members, of which one or more ring atom is a heteroatom such as, but not limited to, N, O, and S. Thus a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms sums up to equal the total number of ring atoms. Ring sizes can also be expressed by the total number of atoms in the ring, e.g., a 3- to 10-membered heterocyclyl group, counting both carbon and non-carbon ring atoms. A heterocyclyl ring can also include one or more double bonds. A heteroaryl ring is an embodiment of a heterocyclyl group. The term “heterocyclyl group” includes fused ring species including those comprising fused aromatic and non-aromatic groups. For example, a dioxolanyl ring and a benzodioxolanyl ring system (methylenedioxyphenyl ring system) are both heterocyclyl groups within the meaning herein. The term also includes polycyclic, e.g., bicyclo- and tricyclo-ring systems containing one or more heteroatom such as, but not limited to, quinuclidyl.

Heterocyclyl groups can be unsubstituted, or can be substituted as discussed above. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed above.

Heteroaryl groups are aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure, which is a multiply-unsaturated cyclic system that contains 4n+2 π electrons wherein n is an integer A heteroaryl group designated as a C₂-heteroaryl can be a 5-ring (i.e., a 5-membered ring) with two carbon atoms and three heteroatoms, a 6-ring (i.e., a 6-membered ring) with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms.

The term “alkoxy” or “alkoxyl” refers to an oxygen atom connected to an alkyl group, including a cycloalkyl group, as are defined above. Examples of linear alkoxy groups include but are not limited to methoxy, ethoxy, n-propoxy, n-butoxy, n-pentyloxy, n-hexyloxy, and the like. Examples of branched alkoxy include but are not limited to isopropoxy, sec-butoxy, tert-butoxy, isopentyloxy, isohexyloxy, and the like. Exemplary alkoxy groups include, but are not limited to, alkoxy groups of 1-6 or 2-6 carbon atoms, referred to herein as C₁₋₆alkoxy, and C₂₋₆alkoxy, respectively. Exemplary alkoxy groups include, but are not limited to methoxy, ethoxy, isopropoxy, etc.

An alkoxy group can include one to about 12-20 carbon atoms bonded to the oxygen atom, and can further include double or triple bonds, and can also include heteroatoms. For example, an allyloxy group is an alkoxy group within the meaning herein. A methoxyethoxy group is also an alkoxy group within the meaning herein, as is a methylenedioxy group in a context where two adjacent atoms of a structure are substituted therewith.

The term “cycloalkoxy” as used herein refers to a cycloalkyl group attached to oxygen (cycloalkyl-O—). Examples of cyclic alkoxy include but are not limited to cyclopropyloxy, cyclobutyloxy, cyclopentyloxy, cyclohexyloxy, and the like. Exemplary cycloalkoxy groups include, but are not limited to, cycloalkoxy groups of 3-6 carbon atoms, referred to herein as C₃₋₆cycloalkoxy groups. Exemplary cycloalkoxy groups include, but are not limited to, cyclopropoxy, cyclobutoxy, cyclohexyloxy, and the like.

The terms “halo” or “halogen” or “halide” by themselves or as part of another substituent mean, unless otherwise stated, a fluorine, chlorine, bromine, or iodine atom, preferably, fluorine, chlorine, or bromine.

A “haloalkyl” group includes mono-halo alkyl groups, poly-halo alkyl groups wherein all halo atoms can be the same or different, and per-halo alkyl groups, wherein all hydrogen atoms are replaced by the same or differing halogen atoms, such as fluorine and/or chlorine atoms. Examples of haloalkyl include trifluoromethyl, 1,1-dichloroethyl, 1,2-dichloroethyl, 1,3-dibromo-3,3-difluoropropyl, perfluorobutyl, and the like.

A “haloalkoxy” group includes mono-halo alkoxy groups, poly-halo alkoxy groups wherein all halo atoms can be the same or different, and per-halo alkoxy groups, wherein all hydrogen atoms are replaced by halogen atoms, such as fluoro. Examples of haloalkoxy include trifluoromethoxy, 1,1-dichloroethoxy, 1,2-dichloroethoxy, 1,3-dibromo-3,3-difluoropropoxy, perfluorobutoxy, and the like.

Standard abbreviations for chemical groups such as are well known in the art are used; e.g., Me=methyl, Et=ethyl, i-Pr=isopropyl, Bu=butyl, t-Bu=tert-butyl, Ph=phenyl, Bn=benzyl, Ac=acetyl, Bz=benzoyl, and the like.

A “salt” as is well known in the art includes an organic compound such as a carboxylic acid, a sulfonic acid, or an amine, in ionic form, in combination with a counterion. For example, acids in their anionic form can form salts with cations such as metal cations, for example sodium, potassium, and the like; with ammonium salts such as NH₄⁺ or the cations of various amines, including tetraalkyl ammonium salts such as tetramethylammonium, or other cations such as trimethylsulfonium, and the like. A “pharmaceutically acceptable” or “pharmacologically acceptable” salt is a salt formed from an ion that has been approved for human consumption and is generally non-toxic, such as a chloride salt or a sodium salt. A “zwitterion” is an internal salt such as can be formed in a molecule that has at least two ionizable groups, one forming an anion and the other a cation, which serve to balance each other. For example, amino acids such as glycine can exist in a zwitterionic form. A “zwitterion” is a salt within the meaning herein. The compounds of the present invention may take the form of salts. The term “salts” embraces addition salts of free acids or free bases which are compounds of the invention. Salts can be “pharmaceutically-acceptable salts.” The term “pharmaceutically-acceptable salt” refers to salts which possess toxicity profiles within a range that affords utility in pharmaceutical applications. Pharmaceutically unacceptable salts may nonetheless possess properties such as high crystallinity, which have utility in the practice of the present invention, such as for example utility in process of synthesis, purification or formulation of compounds of the invention. “Pharmaceutically or pharmacologically acceptable” include molecular entities and compositions that do not produce an adverse, allergic or other untoward reaction when administered to an animal, or a human, as appropriate. For human administration, preparations should meet sterility, pyrogenicity, and general safety and purity standards as required by FDA Office of Biologics standards.

In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described. Moreover, where features or aspects of the invention are described in terms of Markush groups, those skilled in the art will recognize that the invention is also thereby described in terms of any combination of individual members or subgroups of members of Markush groups. Thus, for example, if X is described as selected from the group consisting of bromine, chlorine, and iodine, and Y is described as selected from the group consisting of methyl, ethyl, and propyl, claims for X being bromine and Y being methyl are fully described.

If a value of a variable that is necessarily an integer, e.g., the number of carbon atoms in an alkyl group or the number of substituents on a ring, is described as a range, e.g., 0-4, what is meant is that the value can be any integer between 0 and 4 inclusive, i.e., 0, 1, 2, 3, or 4.

In various embodiments, the compound or set of compounds, such as are used in the inventive methods, can be any one of any of the combinations and/or sub-combinations of the above-listed embodiments.

In various embodiments, a compound as shown in any of the Examples, or among the exemplary compounds, is provided. Provisos may apply to any of the disclosed categories or embodiments wherein any one or more of the other above disclosed embodiments or species may be excluded from such categories or embodiments.

The compounds described herein can be prepared in a number of ways based on the teachings contained herein and synthetic procedures known in the art. In the description of the synthetic methods described below, it is to be understood that all proposed reaction conditions, including choice of solvent, reaction atmosphere, reaction temperature, duration of the experiment and workup procedures, can be chosen to be the conditions standard for that reaction, unless otherwise indicated. It is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated. The starting materials for the examples are either commercially available or are readily prepared by standard methods from known materials. All commercially available chemicals were obtained from Aldrich, Alfa Aesar, Wako, Acros, Fisher, Fluka, Maybridge or the like and were used without further purification, except where noted. Dry solvents are obtained, for example, by passing these through activated alumina columns.

The present invention further embraces isolated compounds of the invention. The expression “isolated compound” refers to a preparation of a compound of the invention, or a mixture of compounds the invention, wherein the isolated compound has been separated from the reagents used, and/or byproducts formed, in the synthesis of the compound or compounds. “Isolated” does not mean that the preparation is technically pure (homogeneous), but it is sufficiently pure to compound in a form in which it can be used therapeutically. Preferably an “isolated compound” refers to a preparation of a compound of the invention or a mixture of compounds of the invention, which contains the named compound or mixture of compounds of the invention in an amount of at least 10 percent by weight of the total weight. Preferably the preparation contains the named compound or mixture of compounds in an amount of at least 50 percent by weight of the total weight; more preferably at least 80 percent by weight of the total weight; and most preferably at least 90 percent, at least 95 percent or at least 98 percent by weight of the total weight of the preparation.

The compounds of the invention and intermediates may be isolated from their reaction mixtures and purified by standard techniques such as filtration, liquid-liquid extraction, solid phase extraction, distillation, recrystallization or chromatography, including flash column chromatography, or HPLC.

Within the present invention it is to be understood that a compound of the formula (I) or a salt thereof may exhibit the phenomenon of tautomerism whereby two chemical compounds that are capable of facile interconversion by exchanging a hydrogen atom between two atoms, to either of which it forms a covalent bond. Since the tautomeric compounds exist in mobile equilibrium with each other they may be regarded as different isomeric forms of the same compound. It is to be understood that the formulae drawings within this specification can represent only one of the possible tautomeric forms. However, it is also to be understood that the invention encompasses any tautomeric form, and is not to be limited merely to any one tautomeric form utilized within the formulae drawings. The formulae drawings within this specification can represent only one of the possible tautomeric forms and it is to be understood that the specification encompasses all possible tautomeric forms of the compounds drawn not just those forms which it has been convenient to show graphically herein.

It will be understood that when compounds of the present invention contain one or more chiral centers, the compounds may exist in, and may be isolated as single and substantially pure enantiomeric or diastereomeric forms or as racemic mixtures. The present invention therefore includes any possible enantiomers, diastereomers, racemates or mixtures thereof of the compounds of the invention.

Typical compositions include a compound of the invention and a pharmaceutically acceptable excipient which can be a carrier or a diluent. For example, the active compound will usually be mixed with a carrier, or diluted by a carrier, or enclosed within a carrier which can be in the form of an ampoule, capsule, sachet, paper, or other container. When the active compound is mixed with a carrier, or when the carrier serves as a diluent, it can be solid, semi-solid, or liquid material that acts as a vehicle, excipient, or medium for the active compound. The active compound can be adsorbed on a granular solid carrier, for example contained in a sachet. Some examples of suitable carriers are water, salt solutions, alcohols, polyethylene glycols, polyhydroxyethoxylated castor oil, peanut oil, olive oil, gelatin, lactose, terra alba, sucrose, dextrin, magnesium carbonate, sugar, cyclodextrin, amylose, magnesium stearate, talc, gelatin, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethylcellulose and polyvinylpyrrolidone. Similarly, the carrier or diluent can include any sustained release material known in the art, such as glyceryl monostearate or glyceryl distearate, alone or mixed with a wax.

The formulations can be mixed with auxiliary agents which do not deleteriously react with the active compounds. Such additives can include wetting agents, emulsifying and suspending agents, salt for influencing osmotic pressure, buffers and/or coloring substances preserving agents, sweetening agents or flavoring agents. The compositions can also be sterilized if desired.

The route of administration can be any route which effectively transports the active compound of the invention to the appropriate or desired site of action, such as oral, nasal, pulmonary, buccal, subdermal, intradermal, transdermal or parenteral, e.g., rectal, depot, subcutaneous, intravenous, intraurethral, intramuscular, intranasal, ophthalmic solution or an ointment, the oral route being preferred.

If a solid carrier is used for oral administration, the preparation can be tableted, placed in a hard gelatin capsule in powder or pellet form or it can be in the form of a troche or lozenge. If a liquid carrier is used, the preparation can be in the form of a syrup, emulsion, soft gelatin capsule or sterile injectable liquid such as an aqueous or non-aqueous liquid suspension or solution.

Injectable dosage forms generally include aqueous suspensions or oil suspensions which can be prepared using a suitable dispersant or wetting agent and a suspending agent Injectable forms can be in solution phase or in the form of a suspension, which is prepared with a solvent or diluent. Acceptable solvents or vehicles include sterilized water, Ringer's solution, or an isotonic aqueous saline solution. Alternatively, sterile oils can be employed as solvents or suspending agents. Preferably, the oil or fatty acid is non-volatile, including natural or synthetic oils, fatty acids, mono-, di- or tri-glycerides.

For injection, the formulation can also be a powder suitable for reconstitution with an appropriate solution as described above. Examples of these include, but are not limited to, freeze dried, rotary dried or spray dried powders, amorphous powders, granules, precipitates, or particulates. For injection, the formulations can optionally contain stabilizers, pH modifiers, surfactants, bioavailability modifiers and combinations of these. The compounds can be formulated for parenteral administration by injection such as by bolus injection or continuous infusion. A unit dosage form for injection can be in ampoules or in multi-dose containers.

The formulations of the invention can be designed to provide quick, sustained, or delayed release of the active ingredient after administration to the patient by employing procedures well known in the art. Thus, the formulations can also be formulated for controlled release or for slow release.

Compositions contemplated by the present invention can include, for example, micelles or liposomes, or some other encapsulated form, or can be administered in an extended release form to provide a prolonged storage and/or delivery effect. Therefore, the formulations can be compressed into pellets or cylinders and implanted intramuscularly or subcutaneously as depot injections. Such implants can employ known inert materials such as silicones and biodegradable polymers, e.g., polylactide-polyglycolide. Examples of other biodegradable polymers include poly(orthoesters) and poly(anhydrides).

For nasal administration, the preparation can contain a compound of the invention, dissolved or suspended in a liquid carrier, preferably an aqueous carrier, for aerosol application. The carrier can contain additives such as solubilizing agents, e.g., propylene glycol, surfactants, absorption enhancers such as lecithin (phosphatidylcholine) or cyclodextrin, or preservatives such as parabens.

For parenteral application, particularly suitable are injectable solutions or suspensions, preferably aqueous solutions with the active compound dissolved in polyhydroxylated castor oil.

Tablets, dragees, or capsules having talc and/or a carbohydrate carrier or binder or the like are particularly suitable for oral application. Preferable carriers for tablets, dragees, or capsules include lactose, corn starch, and/or potato starch. A syrup or elixir can be used in cases where a sweetened vehicle can be employed.

The compounds of the invention can be administered to a mammal, especially a human in need of such treatment, prevention, elimination, alleviation or amelioration of a malcondition. Such mammals include also animals, both domestic animals, e.g. household pets, farm animals, and non-domestic animals such as wildlife.

The compounds of the invention are effective over a wide dosage range. For example, in the treatment of adult humans, dosages from about 0.05 to about 5000 mg, preferably from about 1 to about 2000 mg, and more preferably between about 2 and about 2000 mg per day can be used. A typical dosage is about 10 mg to about 1000 mg per day. In choosing a regimen for patients it can frequently be necessary to begin with a higher dosage and when the condition is under control to reduce the dosage. The exact dosage will depend upon the activity of the compound, mode of administration, on the therapy desired, form in which administered, the subject to be treated and the body weight of the subject to be treated, and the preference and experience of the physician or veterinarian in charge.

Generally, the compounds of the invention are dispensed in unit dosage form including from about 0.05 mg to about 1000 mg of active ingredient together with a pharmaceutically acceptable carrier per unit dosage.

Usually, dosage forms suitable for oral, nasal, pulmonal or transdermal administration include from about 125 μg to about 1250 mg, preferably from about 250 μg to about 500 mg, and more preferably from about 2.5 mg to about 250 mg, of the compounds admixed with a pharmaceutically acceptable carrier or diluent.

Dosage forms can be administered daily, or more than once a day, such as twice or thrice daily. Alternatively dosage forms can be administered less frequently than daily, such as every other day, or weekly, if found to be advisable by a prescribing physician.

### Description

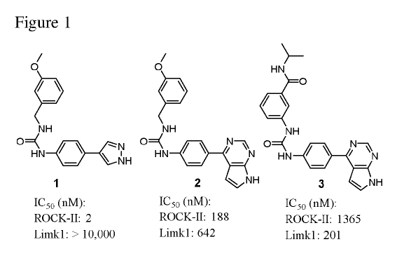

Our group reported a novel pyrazole-phenyl urea scaffold 1 (FIG. 1) as potent and selective Rho kinase (ROCK) inhibitors and their significant intraocular pressure (IOP) lowing effects on rat eyes.^{41, 42} Compound 1 had low Limk inhibition in counter-screen studies (IC₅₀>10 μM). However, SAR investigation revealed that replacement of the hinge-binding moiety pyrazole in 1 with a 4-yl-pyrrolopyrimidine (compound 2) significantly decreased its ROCK-II affinity (ROCK-II IC₅₀=188 nM of 2 vs. 2 nM of 1). On the other hand, compound 2 gained a modest Limk1 inhibition (Limk1 IC₅₀=642 nM vs. >10 μM for 1), revealing an interesting hinge-binder dependent kinase selectivity profile for this phenyl urea based scaffold. Further modification of compound 2 on its urea terminal side led to compound 3 (FIG. 1) which had an even weaker ROCK-II affinity (IC₅₀=1365 nM) but improved Limk1 biochemical potency (IC₅₀=201 nM). Interestingly, the 4-yl-pyrrolopyrimidine moiety in 2 and 3 is also present in Lexicon's piperidine urea/guanidine based Limk inhibitors, and is believed to be involved in hinge-binding interactions.²⁰

Encouraged by the selectivity bias of compound 3 against Limk1 and ROCK-II, we carried out further optimization for this bis-aryl urea scaffold (starting from 3), in the hope to discover highly potent, selective, and proprietary Limk inhibitors for various applications. Herein, we report the synthesis and structure-activity relationship (SAR) studies for this series of bis-aryl urea based Limk inhibitors.

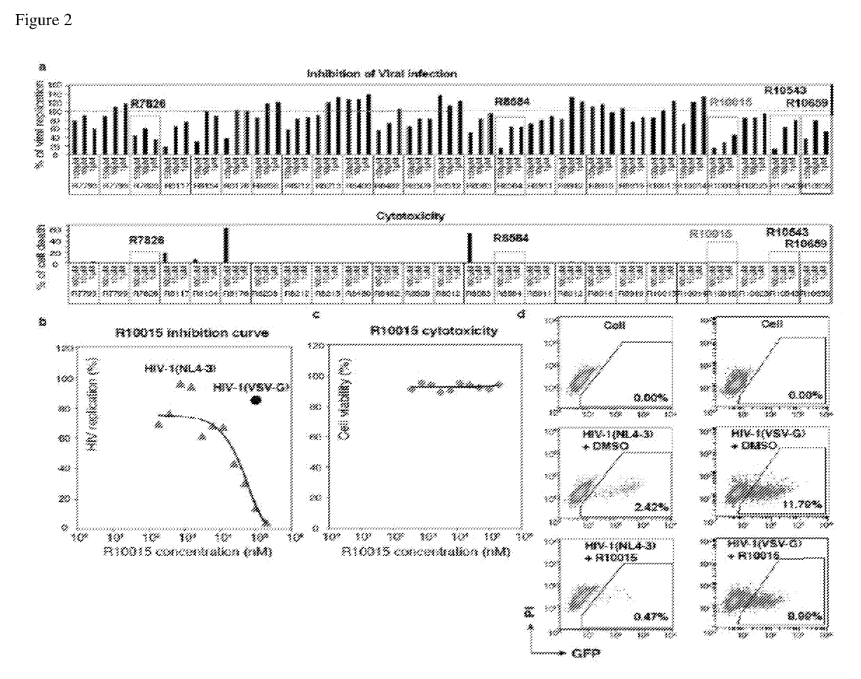

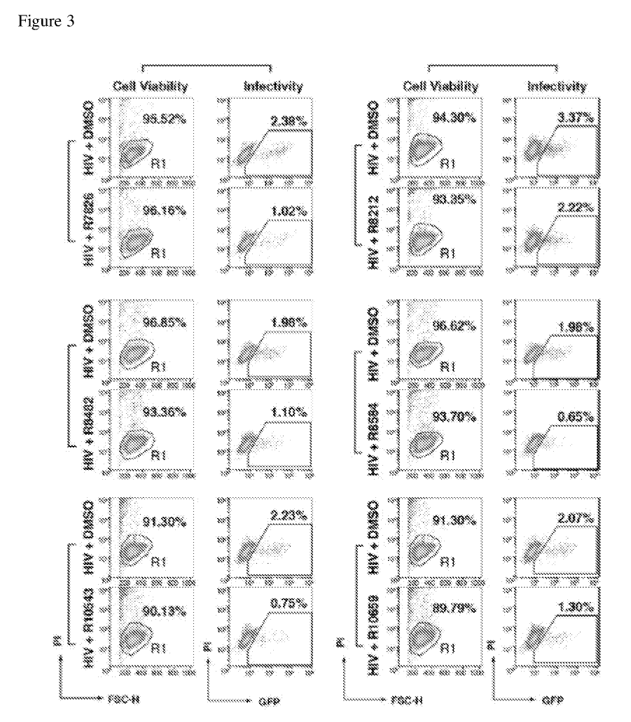

Further, during our preliminary medicinal chemistry efforts, we designed and developed a series of LIMK inhibitors from several scaffolds. We screened approximately 25 of these newly designed LIMK inhibitors using an HIV Rev-dependent indicator cell, Rev-CEM-GFP-Luc, which expressed both GFP and luciferase upon HIV infection. We identified 7 lead compounds (R10015, R8584, R8482, R8212, R7826, R10543, and R10659) that blocked the X4 HIV infection at dosages with no detectable cytotoxicity (FIGS. 2 and 3).

Inhibitors 3 and 7 were accessed through a short route as shown in Scheme 1. Coupling 3-aminobenzoic acid with propan-2-amine gave carbonyl amide 4 in the presence of HATU as coupling reagent and DIEA as base. Mixing intermediate 4 with 1-bromo-4-isocyanatobenzene derivatives in dichloromethane (DCM) produced bromides 5. Finally, targeted inhibitors 3 and 7 were synthesized through a Suzuki coupling with an appropriate aryl boronic acid pinacol ester or alternatively via a two-step palladium catalyzed borylation/Suzuki coupling sequence with an aryl halide. Final targeted Limk inhibitors were all purified by the high pressure reverse-phase liquid chromatograph (HPLC) methodology to give a purity of ≥95% based on UV absorption (254 nm).

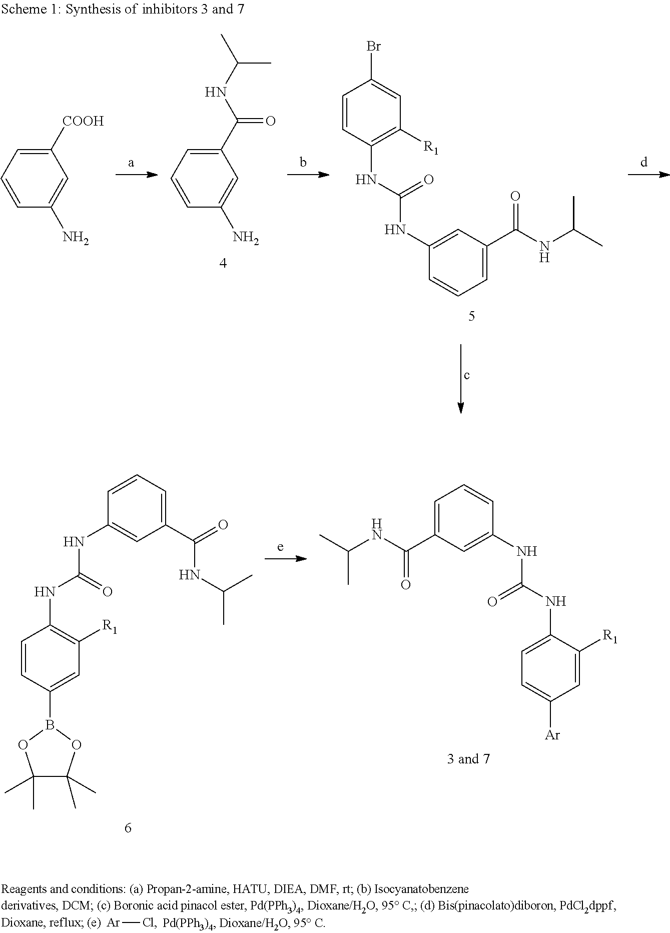

Pyrrolopyrimidines 10 were synthesized through the reaction of substituted anilines 8 with isocyanatobenzene derivatives in DCM at room temperature, followed by Pd-catalyzed borylation/Suzuki coupling reaction with 4-chloro-5-methyl-7H-pyrrolo[2,3-d]pyrimidine (Scheme 2).

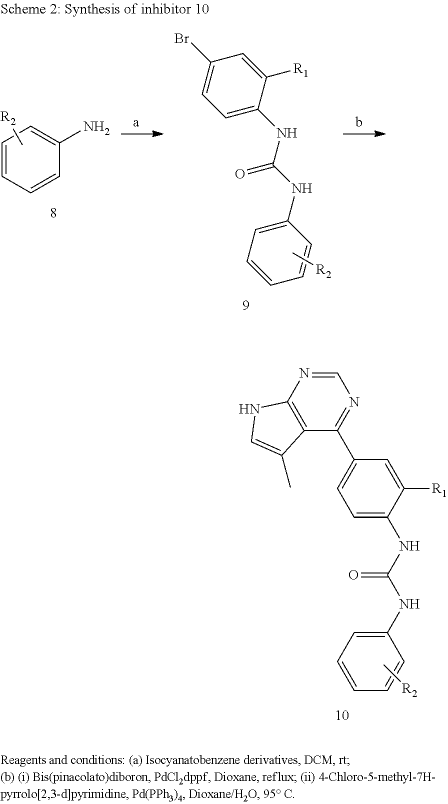

The synthesis of N-substituted (on the urea NH attached to the central phenyl ring) compounds 14 is described in Scheme 3. To make 12b and 12c (12a is commercially available, where a methyl group is attached to the alanine), N-(4-bromophenyl)oxazolidin-2-one 11 was first prepared by reacting 4-bromo-aniline with 2-chloroethyl carbonochloridate in the presence of K₂CO₃ in CH₃CN. Intermediate 11 and a secondary amine pyrrolidine or piperidine were then dissolved in DMSO and heated at 110° C. for 1 h in a microwave reactor to give N-substituted 4-bromo-aniline 12b (from pyrrolidine) and 12c (from piperidine). Mixing 12 and 1-isocyanato-4-methoxybenzene in DCM with stirring gave urea 13. Finally, bromide 13 underwent a Pd-catalyzed borylation/Suzuki coupling reaction sequence to produce 14.

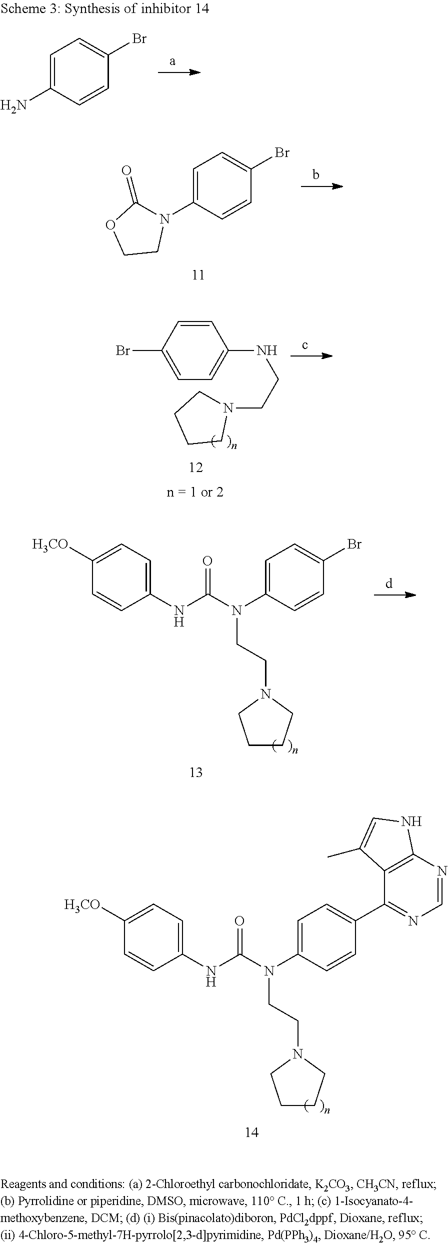

The preparation of N-substituted (on the urea NH attached to the terminal phenyl ring) compound 18 is shown in Scheme 4. Addition of 2-chloroethyl carbonochloridate to a mixture of anilines and pyridine in DCM gave N-phenyl-oxazolidin-2-one derivative 15. Then, refluxing 15 and KOH in EtOH produced N-hydroxylethyl aniline 16. Heating a mixture of iodobenzene, an N′,N′-disubstituted ethanamine, Pd(dba)₂, BINAP, and Cs₂CO₃ in dioxane gave secondary aniline 17. Finally, inhibitors 18 were synthesized from 16 or 17 by following the synthetic procedures described in Scheme 3.

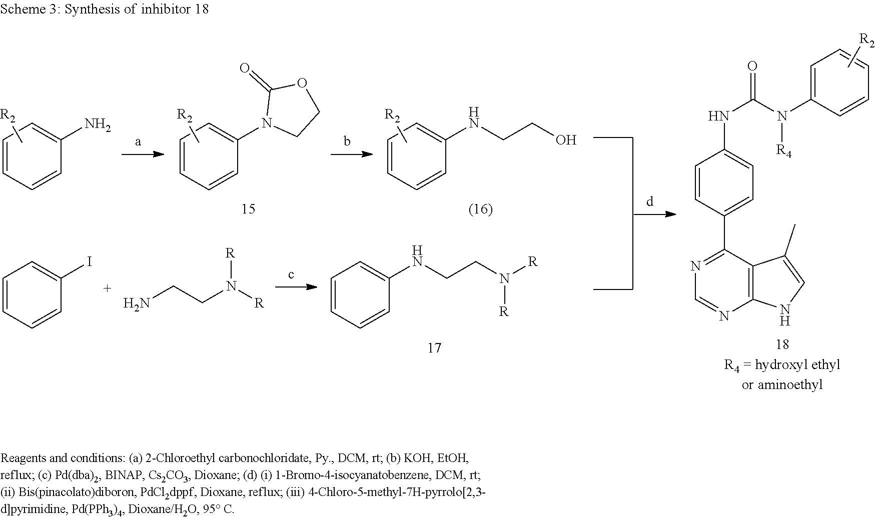

Compounds prepared were first screened in biochemical assays against Limk1 (Reaction Biology Corporation, http://www.reactionbiology.com) and ROCK-II. Selected potent Limk inhibitors were also counterscreened against ROCK-I, PKA, and JNK3, as well as four selected P450 isoforms (1A1, 2C9, 2D6, and 3A4). Potent and selective Limk inhibitors were then evaluated in cell-based assays for their inhibition of cofilin phosphorylation in A7r5 cells. Due to the potential applications of Limk inhibitors for treatment of cancer and HIV-infection, selected lead inhibitors were also assessed in prostate carcinoma (PC-3) cell lines stimulated by hepatocyte growth factor (HGF), and in HIV related CEM-SS T cells using Western blot analysis. To assess the drugability of these bis-aryl urea based Limk inhibitors, a few potent, selective, and membrane permeable compounds were further evaluated in in vitro and in vivo drug metabolism and pharmacokinetics (DMPK) studies.

Since the variation of hinge-binding moieties could induce significant differences in kinase inhibition potency and selectivity, as indicated in FIG. 2, we started SAR studies by varying the heteroaryl ring of 3 in order to discover the best hinge binding moiety for this bis-aryl urea scaffold of Limk inhibitors. As shown in Table 1, compounds with a simple 5- or 6-membered heteroaromatic ring as the hinge-binding moiety, such as pyrazole, pyridine, and aminopyrimidine, are all basically ROCK inhibitors (7a-7c, IC₅₀<200 nM) with low Limk1 inhibition (IC₅₀>10 μM). This observation was in accordance with our previous reports that pyrazole, pyridine, and aminopyrimide were suitable hinge-binding moieties for developing ROCK inhibitors. Application of [5,6]-fused aromatic rings, such as pyrrolopyridine (7d) and purinone (7e) still yielded compounds with good ROCK-II inhibition (IC₅₀=132 and 247 nM for 7d and 7e, respectively) but low Limk1 inhibition (IC₅₀>10 μM). However, the use of a 4-yl-purine moiety reversed the kinase selectivity between ROCK and Limk. Compound 7f exhibited a slightly higher potency for Limk1 inhibition (IC₅₀=1.5 μM) than for ROCK-II inhibition (IC₅₀=5.6 μM). Interestingly, changing hinge-binding group from the purine in 7f to a pyrrolopyrimidine ring in 3 significantly enhanced the Limk1 inhibition potency and the selectivity against ROCK. Moreover, substitution of a methyl group on the 5-position of the pyrrolopyrimidine ring (7g) further improved the inhibition potency over Limk1 (IC₅₀=62 nM vs. 201 nM for 3) and the selectivity against ROCK-II (IC₅₀=1608 nM). Interestingly, application of 6-methyl pyrrolopyrimidine and 5,6-dimethyl pyrrolopyrimidine rings (7h and 7i) gave slightly lower Limk1 inhibition (IC₅₀=80 nM) but better selectivity over ROCK-II. Therefore, further optimizations for other parts of 3 will use 5-methyl pyrrolopyrimidine as the hinge-binding moiety. However, the 5,6-dimethyl pyrrolopyrimidine moiety will also be used in preparing drug candidate Limk inhibitors since it could lead to higher selectivity and better DMPK properties (Tables 5, 6, and 7).

TABLE 1
SAR studies of the hinge-binding moiety.

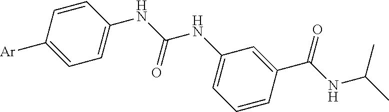

| | | IC₅₀^{a} (nM) | |
|---|---|---|---|
| Cmpd | Ar | Limk1 | ROCK-II |
| 7a | 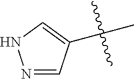 [SMILES: *c1cn[nH]c1] | >10000 | 45 |
| 7b | 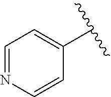 [SMILES: *c1ccncc1] | >10000 | 90 |
| 7c | 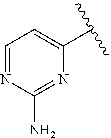 [SMILES: *c1ccnc(N)n1] | >10000 | 166 |
| 7d | 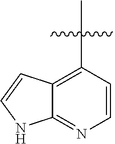 [SMILES: *c1ccnc2[nH]ccc12] | >10000 | 132 |
| 7e | 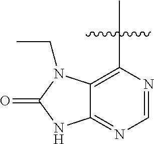 [SMILES: *c1ncnc2[nH]c(=O)n(CC)c12] | >10000 | 247 |
| 7f | 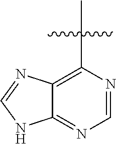 [SMILES: *c1ncnc2[nH]cnc12] | 1527 | 5570 |
| 3 | 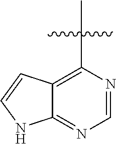 [SMILES: *c1ncnc2[nH]ccc12] | 201 | 1365 |
| 7g | 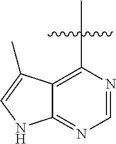 [SMILES: *c1ncnc2[nH]cc(C)c12] | 62 | 1608 |
| 7h | 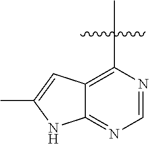 [SMILES: *c1ncnc2[nH]c(C)cc12] | 80 | >10000 |
| 7i | 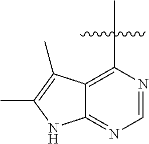 [SMILES: *c1ncnc2[nH]c(C)c(C)c12] | 80 | >10000 |

^{a}IC₅₀ were means of ≥2 experiments with errors within 40% of the mean.

For the convenience of compound synthesis, SAR studies for the central phenyl ring were mainly based on substitutions at its ortho-position (to the urea moiety). As shown in Table 2, three substitutions were evaluated. Compared to the non-substituted inhibitor 7g, the trifluoromethyl substitution yielded a compound (7j) that had a similar Limk1 inhibition potency (IC₅₀=60 nM vs. 62 nM for 7g) but lower selectivity (ROCK-II IC₅₀=976 nM vs. 1608 nM for 7g). However, substitution by a small F group (with a size close to that of a proton, compound 7k) led to both enhanced Limk1 inhibition (IC₅₀=18 nM) and improved selectivity against ROCK-II (based on IC₅₀ values, the selectivity over ROCK-II is 26-fold and 43-fold for 7g and 7k, respectively, Table 2). On the other hand, substitution by a large dimethylaminoethoxy side chain (71) significantly decreased both the Limk1 inhibition (IC₅₀=710 nM vs. 62 nM for 7g) and the selectivity over ROCK-II (Table 2). Therefore, an ortho-F-substitution on the central phenyl ring is the best choice for preparing a highly potent and selective Limk inhibitor.

TABLE 2
Effects of substitutions on the central phenyl ring.

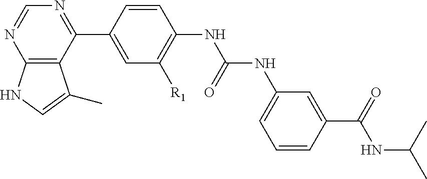

| | | IC₅₀ (nM)^{a} | |
|---|---|---|---|
| Cmpd | R₁ | Limk1 | ROCK-II |
| 7g | H | 62 | 1608 |
| 7j | CF₃ | 60 | 976 |
| 7k | F | 18 | 781 |
| 7l | 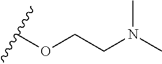 [SMILES: *OCCN(C)C] | 710 | 7083 |

^{a}IC₅₀ were means of ≥2 experiments with errors within 40% of the mean.

SAR was next investigated on the terminal phenyl ring of compound 7g, where a 5-methylpyrrolopyrimidine is used as the hinge-binding moiety and the central phenyl group is non-substituted for the convenience of organic synthesis. As shown in Table 3, removal of the 3-carbonyl amide from the terminal phenyl ring of 7g yielded a compound (10a) with a lower Limk1 inhibitory activity (IC₅₀=142 nM for 10a vs. 62 nM for 7g). Interestingly, replacing the 3-carboxyl amide with a F group (10b) significantly reduced the Limk1 inhibition (IC₅₀=315 nM), which is probably due to special F-bonding interactions between this F group and its surrounding protein residues under the p-Loop (see FIG. 5 of docking studies). This special F-bonding interaction might disturb the optimal binding conformation of these urea based Limk inhibitors. The same effects were also observed in several other Limk inhibitors (see Table 4). It is important to point out that this special effect of F-bonding interactions was not observed for F-substitutions on the central phenyl ring (7k, Table 2), indicating that this effect is dependent on the position of F-substitutions. Actually, we have observed similar negative effects (reducing kinase inhibition potency) of F-bonding interactions in developing our ROCK-II inhibitors and JNK3 inhibitors, where the F-substituted aromatic moieties are all bound to an area under the p-Loop inside the ATP-binding pocket of proteins kinases.

Unlike F-substitutions, replacing the 3-carboxyl amide with a methoxy group resulted in a Limk inhibitor (10d) that had a similar Limk1 inhibitory potency (IC₅₀=75 nM vs. 62 nM for 7g) and a slightly better selectivity over ROCK-II (Table 3). However, the 2-methoxy substitution (10c) significantly reduced the Limk1 inhibition activity (IC₅₀=283 nM). On the other hand, the 4-methoxy substitution (10e) enhanced both Limk1 inhibition (IC₅₀=35 nM) and selectivity against ROCK-II (IC₅₀>10 μM, selectivity >285-fold). Similar SAR patterns were also obtained for F-, Cl-, and methyl-substitutions on this terminal phenyl group (see Table 4), indicating that 4-substitution is the best fit for this scaffold in Limk inhibitions. Heteroaryl rings other than the benzene ring were also evaluated as the terminal aromatic moieties. For example, application of a 2-yl-thiazole (100 resulted in lower Limk1 inhibition (compared to 10a); and the use of a 2-yl-pyridine moiety (10g) almost inactivated the compound against both Limk1 and ROCK-II.

TABLE 3
SAR studies on the terminal aromatic ring
10

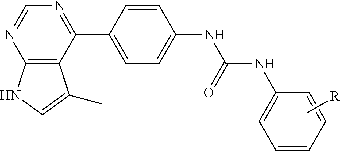

| | | IC₅₀ (nM)^{a} | |
|---|---|---|---|
| Cmpd | R | Limk1 | ROCK-II |
| 10a | H | 142 | 2358 |
| 10b | 3-F | 315 | 5421 |
| 10c | 2-OCH₃ | 283 | 6652 |
| 10d | 3-OCH₃ | 75 | 2572 |
| 10e | 4-OCH₃ | 35 | >10,000 |
| 10f | — | 203 | 2290 |
| 10g | — | 4507 | >10,000 |

^{a}IC₅₀ were means of ≥2 experiments with errors within 40% of the mean.

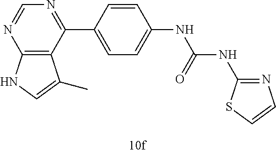 [SMILES: Cc1c[nH]c2ncnc(-c3ccc(NC(=O)Nc4nccs4)cc3)c12]

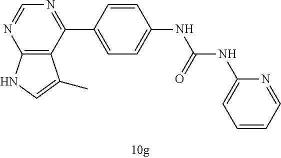 [SMILES: Cc1c[nH]c2ncnc(-c3ccc(NC(=O)Nc4ccccn4)cc3)c12]

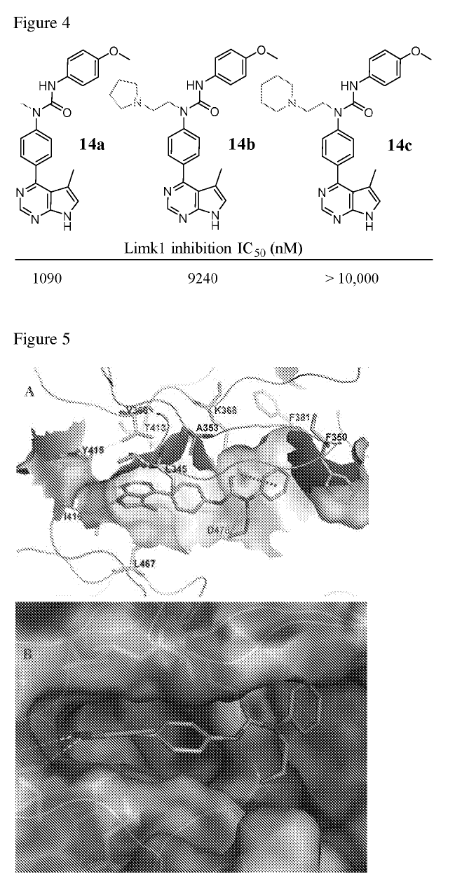

Investigation of the substitution effects on the two urea NH groups was the next focus in our SAR studies. For the urea NH attached to the central phenyl ring, neither small nor large substitutions including pyrrolidinoethyl and piperidinoethyl could be tolerated. As shown in FIG. 4, a simple methyl substitution (14a) would significantly reduce the Limk1 inhibitory potency (IC₅₀=1090 nM vs. 35 nM for 10e). Larger substitutions to this NH group gave even lower Limk1 inhibitions, as evidenced by the Limk1 IC₅₀ values of compounds 14b and 14c (FIG. 4). These results demonstrated that alkylation to this urea NH disturbed the optimal binding conformations, or the NH is involved in H-bonding interactions to the protein, thus resulted in a low Limk affinity (also see docking modes in FIG. 5).

In contrast to observations in FIG. 4, SAR studies demonstrated that substitutions on the urea NH group attached at the terminal phenyl ring were well tolerated, and excellent Limk inhibitors could be obtained through this modification. As shown in Table 4, a pyrrolidinoethyl substitution yielded a compound (18a) with slightly lower Limk1 inhibition (IC₅₀=368 nM vs. 142 nM for 10a). However, replacing the pyrrolidine ring with a hydroxyl group (18b) led to both a high Limk1 inhibitory activity (IC₅₀=43 nM vs. 142 nM for 10a) and a good selectivity over ROCK-II (IC₅₀=6565 nM vs. 2358 nM for 10a). Inspired by 18b, a small library of 4×3=12 analogs (of 18b), based on four functional groups (F—, Cl—, Methyl, and Methoxy) and three substitution patterns on the terminal phenyl ring (2-, 3-, 4-positions), were prepared and evaluated (compounds 18c to 18n, Table 4). Generally, the 4-substitution exhibited the highest and the 2-substitution gave the lowest Limk1 inhibitory activity. Selectivity against ROCK-II followed the same pattern with the 4-substitution being the highest and 2-substitution the lowest, no matter what was the substitution group. Among the 4-substituted Limk inhibitors, the 4-Cl analog had the best Limk1 inhibitory potency (18h, IC₅₀=25 nM) and its 4-F counterpart (18e, IC₅₀=86 nM) had the lowest Limk1 affinity probably due to the special F-bonding interactions, while the Limk1 inhibitory activity and the selectivity against ROCK-II for the methyl and methoxy analogs (18k and 18n) were in between.

To confirm that alkylation to this urea NH group could be well tolerated, two more substitutions were explored. As shown in Table 4, aminoethyl and N′,N′-dimethylaminoethyl substitutions were applied to both 4-Cl- and 4-methoxyphenyl ureas, and the resulting 4 compounds 18o to 18r all exhibited high Limk1 inhibitions. Compounds 18p to 18r were assessed in counter-screen studies, and 18q and 18r were found to have high selectivity against ROCK-II (IC₅₀>10 μM, selectivity is >210-fold and >500-fold for 18q and 18r, respectively) while that for 18p was only ˜21-fold. The lower Limk1 inhibition potency observed for 18a, as compared to 18q and 18r, might be due to its bulky pyrrolidine ring which might have disturbed the optimal binding conformation.

TABLE 4
SAR of the urea NH group attached to the terminal phenyl moiety.

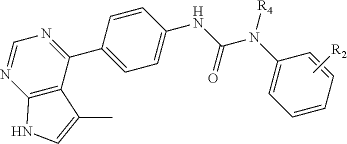

| | | | IC₅₀ (nM)^{a} | |
|---|---|---|---|---|
| Cmpd | R₂ | R₄ | Limk1 | ROCK-II |
| 18a | H | 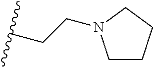 [SMILES: *CCN1CCCC1] | 368 | nd^{b} |
| 18b | H | 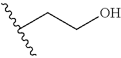 [SMILES: *CCO] | 43 | 6565 |
| 18c | 2-F | 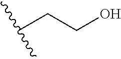 [SMILES: *CCO] | 132 | 1605 |
| 18d | 3-F | 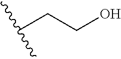 [SMILES: *CCO] | 101 | 1898 |
| 18e | 4-F | 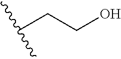 [SMILES: *CCO] | 86 | 3239 |
| 18f | 2-Cl | 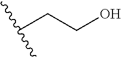 [SMILES: *CCO] | 58 | 3339 |
| 18g | 3-Cl | 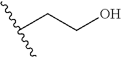 [SMILES: *CCO] | 67 | 11270 |
| 18h | 4-Cl | 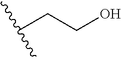 [SMILES: *CCO] | 25 | 4357 |
| 18i | 2-CH₃ | 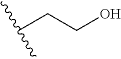 [SMILES: *CCO] | 350 | >10,000 |
| 18j | 3-CH₃ | 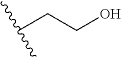 [SMILES: *CCO] | 151 | 8940 |
| 18k | 4-CH₃ | 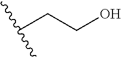 [SMILES: *CCO] | 37 | 5932 |
| 18l | 2-OCH₃ | 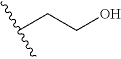 [SMILES: *CCO] | 913 | >10,000 |
| 18m | 3-OCH₃ | 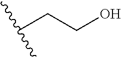 [SMILES: *CCO] | 100 | 3219 |
| 18n | 4-OCH₃ | 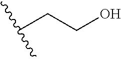 [SMILES: *CCO] | 53 | >10,000 |
| 18o | 4-OCH₃ | 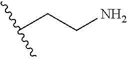 [SMILES: *CCN] | 27 | nd^{b} |
| 18p | 4-Cl | 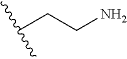 [SMILES: *CCN] | 21 | 460 |
| 18q | 4-OCH₃ | 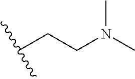 [SMILES: *CCN(C)C] | 47 | >10,000 |
| 18r | 4-Cl | 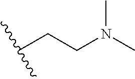 [SMILES: *CCN(C)C] | 20 | >10,000 |

^{a}IC₅₀ were means of ≥2 experiments with errors within 40% of the mean.
^{b}Not determined.

Computer modeling studies of lead compounds demonstrated that these bis-aryl urea based Limk inhibitors are all Type-I ATP-competitive kinase inhibitors. The docking mode of compound 18b in the crystal structure of Limk1 protein (PDB ID 3S95) is shown in FIG. 5. Key interactions in this motif include: two H-bonds between the pyrrolopyrimidine N/NH (N1 and N7) and hinge residue 1416; one plausible H-bond between N3 of pyrrolopyrimidine ring and the side chain OH group of residue T413 (not labeled in FIG. 5 since this H-bonding requires rotation movement of the T413 side chain); one H-bond between the urea carbonyl moiety and the side chain amino group of K368; one H-bond between the OH group and residue D478; cation-π interactions between the terminal phenyl ring and the side chain amino group of K368; hydrophobic interactions between the terminal phenyl ring and its surrounding residues under the P-loop. It is important to point out that hydrophobic interaction between the aromatic rings of pyrrolopyrimidine/central phenyl moieties and their surrounding side chains of protein residues also contributed to the high affinity of these Limk inhibitors.

The binding motif of compound 18b supported our observed SAR. For example, both mono- and bis-methyl substituted (to the 5- and/or 6-position), or even larger group substituted pyrrolopyrimidine rings were well tolerated due to the open space around this area, and these substitutions could enhance the inhibitor's Limk inhibition due to the extra interactions introduced by substitution(s). Substitution to the urea NH attached to the central phenyl ring led to inactive compounds because this substitution could disturb the orientation of the urea carbonyl group thus weakening its H-bonding to K368. On the other hand, substitutions to the urea NH adjacent to the terminal phenyl ring were well tolerated and could lead to enhanced Limk inhibition since there is enough space around this area and the substitution is directed toward the solvent. 4-Substitutions on the terminal phenyl group gave the most active Limk inhibitors (compared to the 2- and 3-substitutions) because there is a deep hydrophobic pocket around there. The H-bonding interaction between the pyrrolopyrimidine N3 and the side chain OH of T413 explained why compound 3 (Table 1) was a good Limk inhibitor while compound 7d had low Limk1 inhibition. The significant decrease of Limk1 inhibition in 7f as compared to 3 (Table 1) is probably due to the extra H-bonding interactions between N5 (of 70 and surrounding protein residues, which might disturb the optimal binding conformation of the ligand thus reduce its affinity toward Limk1.

Our SAR analysis and docking studies for this bis-aryl urea based scaffold of Limk inhibitors showed that both 5- and 6-methyl-4-yl-pyrimidines, and the 5,6-dimethyl-4-yl-pyrrolopyrimidine could serve well as hinge-binding moieties for Limk inhibition. Among them, the 5,6-dimethylpyrrolopyrimidine was the best considering that it could render much better selectivity (against ROCK) and higher microsomal stability (see Table 6). An ortho-F-substitution on the central phenyl ring (to the urea moiety) could improve the Limk inhibitory potency while still keeping high microsomal stability (Table 6). On the other hand, an F-substitution on the terminal phenyl ring reduced inhibitory potency against Limk1 probably due to the special F-bonding interactions (under the P-loop). SAR analysis also indicated that a 4-Cl or a 4-methyl substitution on the terminal phenyl group gave overall best Limk inhibitors. Remarkably, a substitution to the urea NH attached on the terminal phenyl side could improve both biochemical and cell potency, enhance selectivity, and more importantly, increase the inhibitor's DMPK properties and bioavailability (see Table 7 below).

TABLE 5
Biochemical and cell potency for optimized Limk inhibitors. 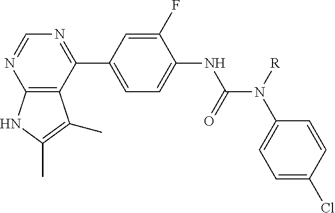
18s-18x

| | | Biochemical IC₅₀ (nM)^{a} | |
|---|---|---|---|
| cmpd | R | Limk1 | ROCK-II |
| 18s | 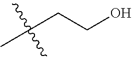 [SMILES: *CCO] | 21 | nd^{b} |
| 18t | 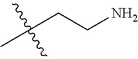 [SMILES: *CCN] | 21 | nd^{b} |
| 18w | 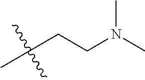 [SMILES: *CCN(C)C] | 19 | >20,000 |
| 18x | 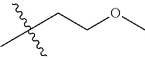 [SMILES: *CCOC] | 8 | >20,000 |

^{a}IC₅₀ were means of ≥2 experiments with errors within 50% of the mean.
^{b}Not determined.

To take advantage of the important SAR information above, Limk inhibitors that combine the best structural elements from SAR analysis were thus prepared and evaluated. Table 5 lists the structures and biochemical potency data for four representative compounds. In compounds 18s to 18w, a 4-yl-5,6-dimethylpyrrolopyrimidine was used as the hinge binding moiety for optimal microsomal stability and better selectivity; An ortho-F-substitution on the central phenyl ring and a 4-Cl substitution on the terminal phenyl group were employed in order to achieve higher Limk1 potency; Representative substitutions on the terminal urea NH were applied to further investigate the DMPK properties (see discussion for Tables 6 and 7). Indeed, these compounds all had excellent Limk1 potency (IC₅₀≤21 nM) and good selectivity against ROCK-II (IC₅₀>20 μM for 18w and 18x).

In order to examine the selectivity profile of these bis-aryl urea based Limk1 inhibitors, selected lead compounds were subjected to counter screening against ROCK-I, JNK3 and four representative cytochrome P450 isoforms. As summarized in Table 6, these Limk inhibitors all exhibited low inhibitory activity over tested kinases and P450 enzymes, except that 7i showed modest inhibition against enzyme 1A2 (77%) at 10 μM. In addition to counterscreens against ROCK and JNK3, lead inhibitor 18b was also profiled against a panel of 61 kinases (Reaction Biology Corporation, http://www.reactionbiology.com/webapps/site/). Results showed that 18b at 1.0 μM inhibited only Limk1 and STK16 with ≥80% inhibition (˜3% hit ratio), and hit also Aurora-a, Flt3, LRRK2, and RET with >50% inhibition (˜10% hit ratio).

These Limk inhibitors also had good to excellent stability in human and rat liver microsomes (Table 6) with good to excellent half-lives. It is important to point out that, compared to the mono-methyl substituted pyrrolopyrimidine based analog 7g, the 5,6-dimethyl pyrrolopyrimidine based Limk inhibitors 7i, 18s, and 18t exhibited a higher stability in both human and rat microsomes, and a higher selectivity against ROCK (see also Tables 2 and 5). However, when the hydroxyl or the amino group on 18s and 18t was methylated, as shown in 18w and 18x, there was a significant drop in the microsomal stability (Table 6). Apparently, the lower stability of 18w and 18x was mainly due to de-methylation on their side chain dimethylamino or methoxy groups. Other important SAR information from the selectivity profiling and stability data in Table 6 include, 1) all hydroxyethyl substituted (to the urea NH) compounds (18 series) had excellent stability in human liver microsomes with the exception of 18g (t_{1/2}=22 min only), 2) F-substitution on the central phenyl ring did not reduce the microsomal stability while still keeping the excellent selectivity (7k vs. 7g), 3) F-substitution on the terminal phenyl ring not only reduced the Limk1 inhibitory potency (compared to its Cl—, methyl, and methoxy substituted counterparts) but also deteriorated the microsomal stability (18e vs. 18b, 18h, 18k, and 18n), 4) 3-substitution on the terminal phenyl ring led to significant reduction of microsomal stability, as compared to its 4-substituted counterpart (18g vs. 18h and 18m vs. 18n), to the non-substituted analog (18b), and even to its 2-substituted analog (180.

TABLE 6
Selectivity, microsomal stability, and cell potency data for selected compounds.

| | Biochemical Inhibition IC₅₀ (nM)^{a} | | | | | |
|---|---|---|---|---|---|---|
| | | | P450% inh. at 10 μM | Microsomal Stability | | Cofilin Phosphorylation |
| | | | 1A2/2C9/ | t_{1/2} (min) | | In A7r5 Cells |
| Cmpd | ROCK-I | JNK3 | 2D6/3A4 | Human | Rat | (IC₅₀, nM)^{a} |
| 7g | 1283 | 7738 | 33/49/16/34 | 33 | 44 | >1000 |
| 7i | >20,000 | nd^{b} | 77/42/34/42 | 47 | >120 | nd^{b} |
| 7k | 2390 | nd^{b} | 29/35/13/32 | 27 | 55 | 4000 |
| 18b | 5536 | nd^{b} | −16/14/5/13 | 87 | 30 | 470 |
| 18e | 3920 | >10,000 | −4/13/3/25 | 44 | 52 | nd^{b} |
| 18f | 4390 | nd^{b} | −4/39/1/37 | 90 | 38 | 420 |
| 18g | 15,050 | nd^{b} | −19/31/2/42 | 22 | 20 | 372 |
| 18h | 5915 | >10,000 | −12/23/4/27 | >120 | 73 | 118 |
| 18k | 7628 | >10,000 | −5/24/−4/5 | 56 | 39 | 190 |
| 18m | 4317 | nd^{b} | 3/17/3/44 | 38 | 21 | nd^{b} |
| 18n | >20,000 | >10,000 | −8/8/−4/2 | 56 | 40 | 730 |
| 18s | nd^{b} | >10,000 | 40/13/8/16 | 99 | 44 | 210 |
| 18t | nd^{b} | >10,000 | 24/24/52/−11 | >120 | >120 | nd^{b} |
| 18w | nd^{b} | nd^{b} | 15/−23/20/−9 | 19 | 23 | 320 |
| 18x | nd^{b} | >10,000 | 23/50/15/52 | 15 | 13 | 250 |

^{a}IC₅₀ were means of ≥ 2 experiments with errors within 40% of the mean. ^{b}Not determined

In an effort to investigate the cell-based activity of these Limk1 inhibitors, we monitored the phosphorylation state of cofilin in several cell lines. Data in A7r5 cells (Table 6) showed that inhibitors without any substitutions on their urea NH group (7g, 7i, 7k) had a cell activity of IC₅₀ values only in the micromolar range. On the other hand, Limk inhibitors with their urea NH group (the one attached to the terminal aryl ring) substituted by a hydroxyethyl, or an aminoethyl, or a methoxyethyl, or a dimethylaminomethyl group (18b to 18x) had IC₅₀ values all in the sub-micromolar range, with the best one close to 100 nM (18h). In addition, SAR patterns shown in the cell-based potency were similar to those observed in biochemical potency and selectivity assays. For example, 4-Cl (18h) and 4-methyl (18k) substitutions produced compounds with better cell activity than 4-methoxy (18n) substitutions, and the 4-substitution exhibited the highest cell activity among 2-, 3-, and 4-substitutions (18f, 18g, and 18h) on the terminal phenyl ring.

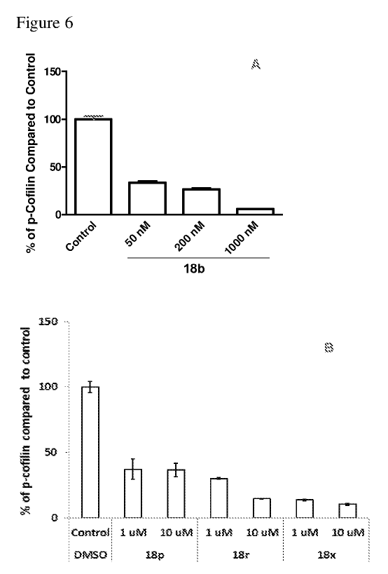

Since Limk inhibitors could find wide applications, such as in glaucoma, cancer, infection, and Alzheimer's disease (AD) etc., cofilin phosphorylation assays were also carried out for a few selected lead compounds in prostate carcinoma (PC3) cell lines stimulated by hepatocyte growth factor (HGF) and in HIV-related CEM-SS T cell lines. As shown in FIG. 6, inhibitor 18b exhibited significant inhibition even at a concentration of only 50 nM in Western blot analysis of cofilin phosphorylation in PC-3 cells (FIG. 6A). Similar cell-based potency was also observed for 18f and 18h in PC-3 cells. The phosphorylation status of p-cofilin in CEM-SS T cells for inhibitors 18p, 18r, and 18x is shown in FIG. 6B. Again >50% inhibition was seen for all these compounds at 1 μM, an inhibitory potency similar to that obtained in A7r5 cells. The results from these three tested cell lines demonstrated that the optimized Limk inhibitors had good cell permeation. Compounds 18p and 18r had almost the same biochemical Limk1 potency (IC₅₀ values were both ˜20 nM, Table 4). Apparently, the better cell potency observed for 18r than for 18p (FIG. 6B) is due to the free NH₂ group present in 18p, a structural element normally associated with deteriorated cell penetration.

In vivo pharmacokinetics (PK) studies were conducted for selected compounds during the whole optimization at various stages in order to identify structural elements that are favorable for in vivo applications, and/or to evaluate the feasibility of optimized Limk inhibitors for animal studies. PK properties of iv dosing (1 mg/kg) and the oral bioavailability (% F) for selected lead Limk inhibitors are listed in Table 7. Generally, a 2-hydroxyethyl side chain reduced the clearance (Cl) compared to the non-substituted (NH) urea derivatives (10a, 18b, 18h, 18k, 18n vs. 7g and 7k). In contrast, a side chain containing a terminal amino group increased the clearance significantly (18o, 18p, 18r vs. 18h, 18k, and 18n). Remarkably, the high clearance of compounds with an amino side chain could be reduced dramatically by introducing an F-substitution on the central phenyl ring, or by using a 5,6-dimethylpyrrolopyrimidine (instead of the 5-methylpyrrolopyrimidine) as the hinge-binding moiety, or a combination of both (18w vs. 18r). All Limk1 inhibitors listed in Table 7 had reasonable volume of distribution (Vd) values except a few which possessed an amino side chain and in which a 5-methylpyrrolopyrimidine was used as the hinge binding moiety (18o, 18p, and 18r). The much lower Cl and Vd values and higher AUC value for 18w as compared to those for 18r (and also for 18o and 18p) further demonstrated that an F-substitution on the central phenyl ring and the application of a 5,6-dimethylpyrrolopyrimidine as the hinge-binding moiety can improve the inhibitor's PK properties.

TABLE 7
Data for Plasma pharmacokinetics studies on rats.^{a}

| | Cl (iv) | | T_{1/2} | AUC | Cmax | F |
|---|---|---|---|---|---|---|
| | (mL/ | Y_{d} (iv) | (iv) | (iv) | (iv) | (%)^{c} |
| Cmpd | min/kg)^{b} | (L/kg)^{b} | (h)^{b} | (μM*h)^{b} | (μM)^{b} | (po) |
| 7g | 9.0 | 0.3 | 1.0 | 4.3 | 8.5 | 0 |
| 7k | 7.1 | 0.7 | 1.5 | 5.5 | 5.1 | 0 |
| 10a | 3.4 | 0.5 | 1.6 | 14.3 | 7.0 | 16 |
| 18b | 5.2 | 0.4 | 2.2 | 8.4 | 7.7 | 36 |
| 18f | 7.7 | 0.7 | 1.5 | 5.1 | 4.7 | 21 |
| 18h | 3.0 | 0.5 | 2.6 | 13.2 | 10.5 | 20 |
| 18k | 2.7 | 0.3 | 1.8 | 15.6 | 9.4 | 24 |
| 18n | 2.1 | 0.3 | 2.2 | 19.4 | 10.6 | 24 |
| 18o | 36.0 | 6.0 | 4.6 | 1.1 | 2.2 | 0 |
| 18p | 55.9 | 4.0 | 5.1 | 0.7 | 1.1 | 0 |
| 18r | 42.2 | 17.1 | 6.1 | 0.6 | 0.2 | 0 |
| 18s | 4.5 | 0.5 | 3.2 | 14.5 | 12.3 | 29 |
| 18w | 14.2 | 1.5 | 3.3 | 3.0 | 2.4 | nd^{d} |
| 18x | 6.9 | 1.2 | 3.4 | 5.1 | 3.5 | nd^{d} |

^{a}Data reported were the mean of three determinations, and the standard error was within 40% of the mean. ^{b}iv dosing: 1 mg/kg. ^{c}po dosing: 2 mg/kg. ^{d}Not determined.

The PK data in Table 7 showed that substitution to the urea NH group could generally increase the half-lives of these urea based Limk inhibitors (the 10 and 18 series vs. the 7 series). The AUC and Cmax properties for these compounds were also excellent except for the three inhibitors (18o, 18p, and 18r) which contained an amino side chain and no F-substitutions on their central phenyl ring and in which a 5-methyl pyrrolopyrimidine was used as the hinge-binding moiety. It is important to point out that, even with an amino side chain, inhibitor 18w still exhibited good AUC and Cmax values, probably due to the presence of both an F-substitution on the central phenyl ring and a 5,6-dimethylpyrrolopyrimidine moiety in its structure. Data in Table 7 also indicated that, while the non-substituted urea compounds (7g and 7k) had no oral bioavailability (% F) at all, all inhibitors containing a hydroxyethyl side chain could exhibit reasonable oral bioavailability. However, those inhibitors containing an amino side chain (18o, 18p, and 18r) had no oral bioavailability either, probably because of the high clearance (Cl) exhibited by these compounds.

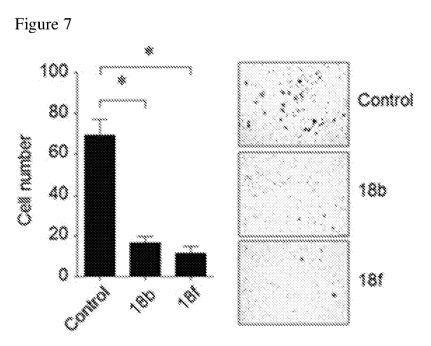

Since Limk1 expression is highly expressed in cancerous prostate cells and predominantly found in metastatic prostate tumor tissues, and is required for cancer cell migration and invasion, Limk1 is considered as a biomarker for prostate cancer progression. Limk1 is involved in Rac-induced actin cytoskeleton reorganization through inactivating phosphorylation of cofilin, and also mediated with focal adhesion complexes. Reorganization of cytoskeleton is an essential feature of motility, detachment, and invasion of cancer cells. Moreover, Limk1 expression is correlated with the aggressiveness of cancer cells, and Limk1 expression in metastatic PC-3 cells is higher than less-aggressive LNCaP and M21 cells. To confirm the role of Limk inhibitors on the invasion and migration of prostate cancers, we examined the effect of optimized Limk inhibitors in PC-3 cells using an in vitro invasion assay or in vitro migration assay. Thus, Transwell chambers were coated with GFR Matrigel, and PC-3 cells were seeded in the insert of the chamber as described in the Experimental Section. After incubating for 48 hours, the invasive PC-3 cells were counted and analyzed by hematoxylin staining under microscope. As shown in FIG. 7 for two representative inhibitors 18b or 18f, the invasion of PC-3 cells was significantly inhibited by the treatment of 1 μM Limk inhibitors (76% for 18b, and 83% for 18f, compared to the control).

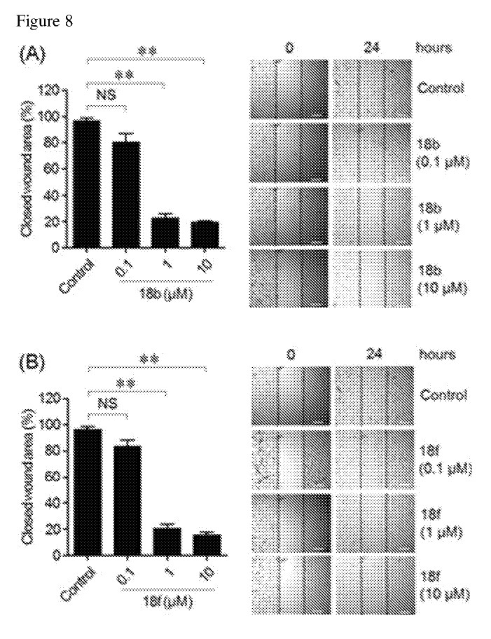

To verify the role of Limk inhibitors on migration of PC-3 cells, a wound was created by scratching in a cell monolayer as described in the Experimental Section. After incubating for 24 hours with treatment of inhibitors 18b or 18f, the closed wound area, indicating migrated cells, was analyzed by ImageJ software (Ver 1.48). As shown in FIG. 8, the migrated PC-3 cells were decreased significantly even at a concentration as low as 0.1 μM, and the migration was inhibited 74% by 1 μM of inhibitor 18b (16.5% (NS) by 0.1 μM, 74.0% (p<0.01) by 1 μM, and 77.5% (p<0.01) by 10 μM compared to the control). Similar inhibition potency was also obtained for inhibitor 18f (13.0% (NS) by 0.1 μM, 76.1% (p<0.01) by 1 μM, and 81.0% (p<0.01) by 10 μM, compared to the control). These results indicated that 18b or 18f had inhibitory effects on invasion and migration of metastatic PC-3 cells. Considering that both inhibitors had low inhibition against ROCK-I and ROCK-II (Tables 4 and 6), the inhibition of which could also lead to suppression of cell migration/invasion, results in FIGS. 7 and 8 also demonstrated that 18b and 18f must have played a role in Limk inhibitions in vitro.

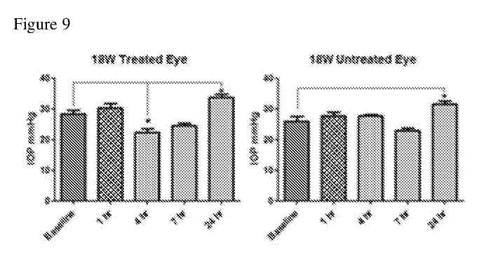

To demonstrate the potential application of these Limk inhibitors for the treatment of glaucoma, the intraocular pressure (IOP)-lowering effect of compound 18w was monitored after applying it topically on rat eyes (Brown Norway rats, n=6/group, housed under constant low-light conditions) followed a protocol described previously by our groups. Thus, compound 18w was applied to the right eyes of an elevated IOP rat model (initial IOP was ˜28 mmHg) using a dose of 50 μg (20 μL drop of a 0.25% solution). As shown in FIG. 9, significant decreases in IOP were detected at 4 h, slightly weakened at 7 h, and IOP returning to baseline at 24 h as compared to the vehicle. It must be pointed out that the IOP drop could not be due to ROCK inhibition since 18w had a high selectivity against ROCK (Table 5).

Through the application of a 4-yl-pyrrolopyrimidine as the hinge-binding moiety to replace the pyrazole group in ROCK inhibitor 1, we identified compounds with high Limk1 inhibition potency. Systematic SAR studies around this bis-aryl urea scaffold (3) have led to a series of potent and selective Limk inhibitors. Docking studies demonstrated that these bis-aryl urea Limk inhibitors exhibited a typical Type-I kinase binding motif. The optimized Limk inhibitors had high biochemical potency and high selectivity over ROCK-I, ROCK-II, and JNK3. Inhibitor 18b (also coded as SR-7826) was found to hit only Limk1 and STK16 with ≥80% inhibition at 1 μM against a panel of 61 kinases. The lead Limk inhibitors also had good cell-based potency in cofilin phosphorylation assays and in cell-based migration/invasion assays. In addition, they had fair to excellent in vitro and in vivo DMPK properties, such as a clean inhibition profile against select CYP-450 isoforms, a high stability in human and rat liver microsomes, and favorable PK properties in iv dosing (high AUC/Cmax, low Cl, and long half-lives) and fair to good oral bioavailability (18b, 18k, 18n, and 18s) in rats. For example, compounds 18s to 18x (also coded as SR-11157) all had excellent potency against Limk1 (IC₅₀s≤21 nM), good cell-based activity against cofilin phosphorylation in A7r5 cells (IC₅₀s≤320 nM), and high selectivity over ROCK and JNK. The optimized inhibitors, such as 18b and 18f, showed excellent activities in migration/invasion cell-based assays. In addition, significant IOP drop on rat eyes (>20%) was achieved for inhibitor 18w (also coded as SR-11124) after topical administration (at a dose of 50 μg).

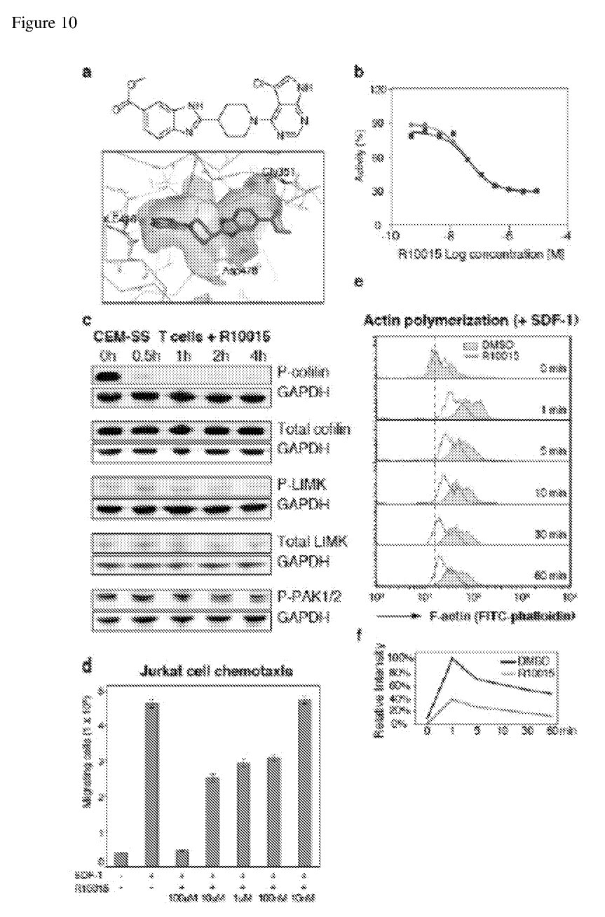

Further, lead LIMK inhibitors have been identified from three chemotypes. Scaffold A is based on the 4-yl-piperidine- or piperazine-benzimidazole derivatives; scaffold B is derived from phenylbenzimidazole analogs, and scaffold C is based on the bis-aryl urea moiety. All three scaffolds share a substituted or un-substituted 7H-pyrrolo[2,3-d] pyrimidine moiety (Table 10). Molecular docking studies in a crystal structure of LIMK1 (PDB ID 3S95) demonstrated that this pyrrolopyrimidine moiety functions as an ATP-site hinge binding group bound to the backbone of hinge residue Ile416 with H-bonding interactions from the NH/N of pyrrolopyrimidine. All compounds from these three scaffolds are Type-I ATP-competitive kinase inhibitors (FIG. 9). In addition to hinge interactions, the terminal aromatic moiety of these LIMK inhibitors is bound to a pocket under the P-loop with strong hydrophobic interactions (FIG. 10). Additional H-bonding interaction(s), which could contribute to the high affinity of these LIMK inhibitors, might also present in this binding motif depending on the functional substitution(s). For example, for R10015, there is also an H-bonding interaction between the ester carbonyl group and the backbone amide of residue G351 (FIG. 10).

TABLE 8
Structures of representative compounds and LIMK1 IC₅₀ values (nM)

| | | | LIMK1 |
|---|---|---|---|
| Compound | Scaffold | Structure | IC₅₀ (nM) |
| R8212 | A | 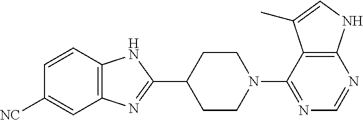 [SMILES: Cc1c[nH]c2ncnc(N3CCC(c4nc5cc(C#N)ccc5[nH]4)CC3)c12] | 44 ± 6 |
| R8482 | A | 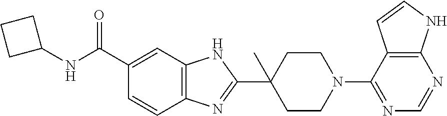 [SMILES: CC1(c2nc3ccc(C(=O)NC4CCC4)cc3[nH]2)CCN(c2ncnc3[nH]ccc23)CC1] | 41 ± 4 |
| R10015 | A | 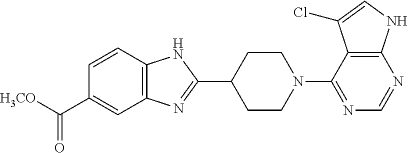 [SMILES: COC(=O)c1ccc2[nH]c(C3CCN(c4ncnc5[nH]cc(Cl)c45)CC3)nc2c1] | 38 ± 5 |
| R10543 | A | 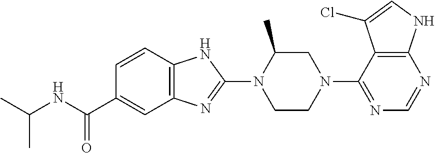 [SMILES: CC(C)NC(=O)c1ccc2[nH]c(N3CCN(c4ncnc5[nH]cc(Cl)c45)C[C@@H]3C)nc2c1] | 5 ± 2 |
| R10659 | A | 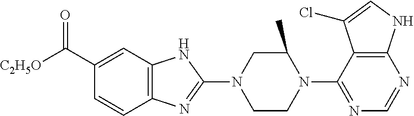 | 45 ± 6 |
| R8584 | B | 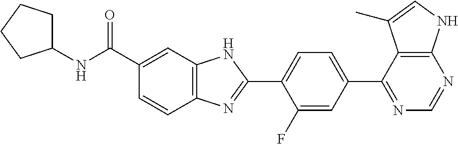 [SMILES: Cc1c[nH]c2ncnc(-c3ccc(-c4nc5ccc(C(=O)NC6CCCC6)cc5[nH]4)c(F)c3)c12] | 50 ± 7 |
| R7826 | C | 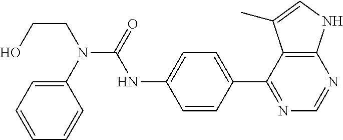 [SMILES: Cc1c[nH]c2ncnc(-c3ccc(NC(=O)N(CCO)c4ccccc4)cc3)c12] | 43 ± 4 |

We selected R10015 for further detailed mechanistic studies. The synthesis, purification, and biochemical characterization of R10015 were described in the Examples section and in FIG. 10. The biochemical IC₅₀ value of R10015 against purified human LIMK1 kinase activity was determined to be approximately 38 nM (FIG. 10b). Profiling against a panel of 62 kinases (Table 9) demonstrated that R10015 is highly selective with significant off-target inhibition against only LRRK2 and p70S6K (≥90% inhibition at 1 μM of R10015), and moderate inhibition over PKA (˜76%), ROCK2 (˜70%), and FLT3 (˜68%).

To further confirm the specificity of R10015 in cells, we treated human CEM-SS T cells for a time course, and detected a drastic inhibition of cofilin serine 3 phosphorylation (FIG. 2c). This inhibition is specific to the kinase activity of LIMK, as R10015 did not alter the total amount of cofilin or LIMK in cells. R10015 also did not inhibit the phosphorylation of LIMK itself by other kinases such as PAK1/2, whose activity is regulated by upstream Rho family GTPases such as Rac, cdc42, and Rho 4,22. The phosphorylation of PAK1/2 itself was also not inhibited by R10015 (FIG. 10c), demonstrating that upstream kinases and GTPases are not affected by R10015.

We further tested R10015 for inhibiting SDF-1-mediated chemotaxis and actin dynamics, which are known to be regulated by LIMK. We observed dosage-dependent inhibition of Jurkat T cell chemotaxis, and the IC50 is around 10 μM (FIG. 10d). Consistently, pre-treatment of resting CD4 T cells with R10015 markedly depressed SDF-1-mediated actin polymerization (FIGS. 10e and 10f), confirming that R10015 blocks LIMK-regulated actin dynamics. For inhibiting HIV infection of CD4 T cells, R10015 showed a half maximal inhibition concentration (IC50) of 14.9 μM, and the drug had no detectable cytotoxicity at all the concentrations tested (up to 200 μM) (FIGS. 9b and 9c). In addition, R10015 did not inhibit VSVG pseudotyped HIV even at 100 μM (FIGS. 9b and 9d), demonstrating that the inhibition of HIV did not result from non-specific cytotoxicity, and is indeed specific to viral processes related to HIV gp120-mediated fusion and entry, which have been known to require cortical actin dynamics 2; the VSV-G-mediated endocytosis bypasses the cortical actin, and thus is less susceptible to R10015. These results support that R10015 inhibits HIV through direct blockage of LIMK-regulated actin dynamics.

The drug can also be used for preventing sexually transmitted diseases (STD). It was found that R10015 also inhibited Herpes simplex virus. The drug can be formulated for topical use to prevent the sexual transmission of STD-causing viruses. In addition, it can also inhibit bacterial infection by blocking intracellular bacterial migration that relies on actin polymerization and LIMK activity. For example, Chlamydia is a sexually transmitted bacterial infection that can be treated with a compound of the invention.

TABLE 9
Profiling data study for R10015 at 1 μM against 60 kinases

| Kinase | % Activity |
|---|---|
| ABL1 | 89.2 |
| AKT2 | 73.1 |
| ALK | 95.5 |
| AMPK(A1/B1/G1) | 76.6 |
| Aurora A | 60.3 |
| BRAF | 91.9 |
| BTK | 90.2 |
| CAMK2b | 90.8 |
| CDK2/cyclin A | 82.7 |
| CDK5/p35 | 85.5 |
| CK1d | 85. |
| CK2a | 94.2 |
| c-Kit | 81.3 |
| c-MET | 82.1 |
| c-5rc | 92.4 |
| DAPK1 | 87.8 |
| EGFR | 93.2 |
| EPHA3 | 91.1 |
| ERK2/MAPK1 | 87.2 |
| FAK/PTK2 | 81.5 |
| FGFR1 | 86.5 |
| FLT3 | 32.2 |
| G5K3b | 67.8 |
| IGFIR | 90.0 |
| IKKa/CHUK | 90.5 |
| IKKb/IKBKB | 85.1 |
| IKKe/IKBKE | 84.1 |
| JAK3 | 61.1 |
| JNK1 | 95.2 |
| JNK3 | 85.7 |
| KDR/VEGFR2 | 87.2 |
| LIMK1 | 6.4 |
| LRRK2 (G20195) | 6.7 |
| MEK1 | 75.6 |
| MLCK/MYLK | 83.3 |
| MRCKa/CDC42BPA | 96.5 |
| mTOR/FRAP1 | 100.0 |
| NEK1 | 67.2 |
| P38a/MAPK14 | 98.6 |
| p70S6K/RPS6KB1 | 10.0 |
| PAK2 | 98.2 |
| PDGFRb | 78.6 |
| PIM2 | 97.6 |
| PKA | 24.0 |
| PKCa | 47.5 |
| PKN1/PRK1 | 58.6 |
| PLK2 | 103.2 |
| RET | 46.5 |
| ROCK1 | 42.3 |
| ROCK2 | 29.5 |
| R5K1 | 47.2 |
| SGK1 | 61.0 |
| SLK/STK2 | 108.0 |
| STK16 | 48.6 |
| STK33 | 56.5 |
| TAK1 | 82.4 |
| TAOK1 | 54.0 |
| TBK1 | 82.0 |
| TESK1 | 90.2 |
| TLK1 | 98.5 |
| WEE1 | 97.2 |
| ZIPK/DAPK3 | 98.5 |

Profiling was carried out at Reaction Biology Corporation following a protocol described at its website: http://www.reactionbiology.com/webapps/site/. The % data were the average from two determinations. ATP concentration used: 10 μM; Staurosporine was used as the positive control.

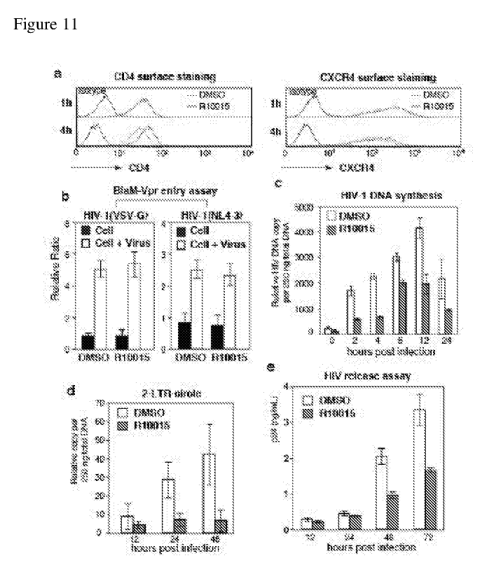

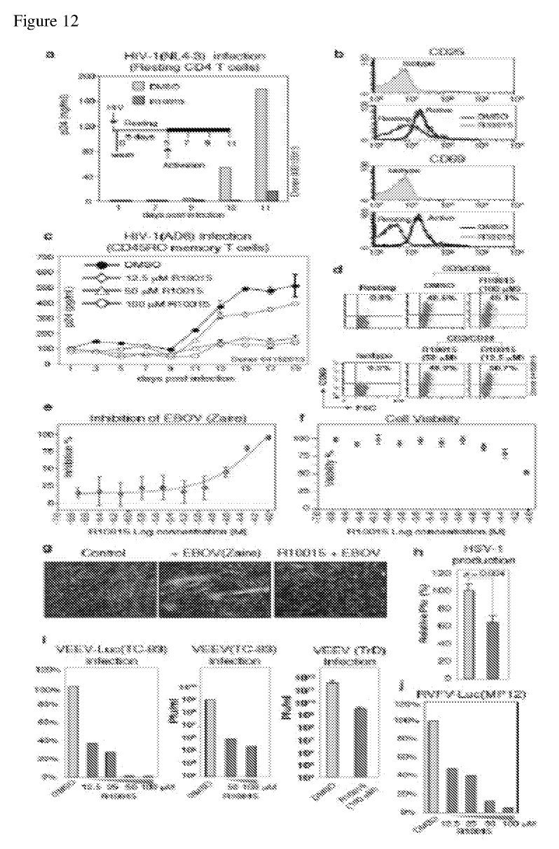

To further elucidate the anti-HIV mechanism, we pre-treated human CEM-SS T cells with R10015 for 1 hour. This brief treatment did not alter the surface expression of CD4 and CXCR4; prolonged treatment (4 hours), however, slightly decreased the surface density of CD4 (FIGS. 11a and 11b) 4. R10015 did not significantly inhibit viral entry, as measured by infection with HIV-1 Vpr-β-lactamase (FIG. 1c) or Nef-luciferase tagged virion particles. However, when post-entry steps were measured using a single-cycle HIV-1(Env) (pseudotyped with HIV-1 gp160), R10015 inhibited viral DNA synthesis at all time points (FIG. 11c). In addition, R10015 inhibited viral nuclear migration, as measured by viral 2-LTR circles (FIG. 11d). Furthermore, R10015 also inhibited viral release when applied at a later stage of HIV infection (FIG. 11e). These results demonstrate that R10015 inhibited viral DNA synthesis, nuclear migration, and virion release, a phenotype consistent with the inhibitions observed in shRNA LIMK1 knockdown cells.

During HIV transmission, early viruses utilize CCR5 (R5), and mainly infect memory CD4 T cells and macrophages, whereas late viruses also utilize CXCR4 (X4). We tested R10015 for inhibiting X4 and R5 viral infection of blood resting CD4 T cells and memory CD4 T cells, and observed inhibition of X4 and R5 viruses at non-toxic dosages (FIGS. 12a to 12d). These results confirmed that the LIMK inhibitor is effective in blocking HIV infection of primary targets. Given that the requirement for actin dynamics is shared among multiple viruses, we tested R10015 for its ability to inhibit several other viruses, and found that R10015 also inhibited EBOV (Zaire ebolavirus), RVFV (rift valley fever virus), VEEV (venezuelan equine encephalitis virus), and HSV-1 (Herpes simplex virus) (FIGS. 12e to 12j), demonstrating its broad anti-viral activity.

Our study demonstrated the possibility of developing highly specific LIMK inhibitors for inhibiting HIV and other viral infection. Given the lack of an effective HIV vaccine, these novel inhibitors are valuable alternatives for preventing HIV sexual transmission. These LIMK inhibitors inhibit viral reverse transcription, nuclear migration, and release, and are expected to be broad spectrum for HIV stains, because of the highly conserved nature of viral dependence on actin dynamics for infection. In addition, these LIMK inhibitors have anti-inflammatory properties, and can inhibit the migration and chemotaxis of infected immune cells for HIV cell-cell transmission (FIG. 10d). These virological and immunological properties make them ideal candidates for pre-exposure prophylaxis that complements the current anti-retroviral drugs.

### Examples

Commercially available reagents and anhydrous solvents were used without further purification unless otherwise specified. Thin layer chromatography (TLC) analyses were performed with precolated silica gel 60 F254. The mass spectra were recorded by LC/MS with Finnigan LCQ Advantage MAX spectrometer of Thermo Electron®. Flash chromatography was performed on prepacked columns of silica gel (230-400 Mesh, 40-63 μm) by CombiFlash® with EtOAc/hexane or MeOH/DCM as eluent. The preparative HPLC was performed on SunFire C₁₈ OBD 10 μm (30×250 mm) with CH₃CN+50% MeOH/H₂O+0.1% TFA as eluent to purify the targeted compounds. Analytic HPLC was performed on Agilent technologies 1200 series with CH₃CN (Solvent B)/H₂O+0.9% CH₃CN+0.1% TFA (Solvent A) as eluent and the targeted products were detected by UV in the detection range of 215-310 nm. All compounds were determined to be >95% pure by this method. NMR spectra were recorded with a Bruker® 400 MHz spectrometer at ambient temperature with the residual solvent peaks as internal standards. The line positions of multiplets were given in ppm (δ) and the coupling constants (J) were given in Hertz. The high-resolution mass spectra (HRMS, electrospray ionization) experiments were performed with Thermo Finnigan orbitrap mass analyzer. Data were acquired in the positive ion mode at resolving power of 100000 at m/z 400. Calibration was performed with an external calibration mixture immediately prior to analysis.

### General Synthetic Procedures:

The mixture of 3-aminobenzoic acid (10 mmol), propan-2-amine (10 mmol), HATU (10 mmol), and DIEA (30 mmol) in DMF (10 mL) was stirred at room temperature until the complete conversion of the started material. Then, saturated NaHCO₃ was added to quench the reaction and extracted with ethyl acetate (3×15 mL). The organic layers were combined, dried over anhydrous Na₂SO₄ and concentrated in vacuo to give crude aniline carboxamide 4. Aniline 4 (0.2 mmol) was then added to the solution of isocyanatobenzene derivatives (0.2 mmol) in DCM (1 mL). The mixture was stirred at room temperature for 2 h. Then, the solvent was removed in vacuo to give the crude bromide 5 for next step without further purification. The mixture of substituted anilines 8 (0.2 mmol) and isocyanatobenzene derivatives (0.2 mmol) in DCM (1 mL) was stirred at room temperature for 2 h, then the solvent was removed in vacuo to give the crude bromides 9 for next step without further purification.

2-Chloroethyl carbonochloridate (10 mmol) was added to a mixture of 4-bromo-aniline (10 mmol) and K₂CO₃ (30 mmol) in CH₃CN (100 mL) and the reaction was stirred for 24 h. Then, solvent was removed in vacuo and the remaining residue redissolved in water and ethyl acetate. The organic layers were combined, dried over anhydrous Na₂SO₄, concentrated in vacuo, and purified through silica gel to give crude N-(4-bromophenyl)oxazolidin-2-one 11.

Then 11 (0.2 mmol) and secondary amine (0.6 mmol) including pyrrolidine and piperidine were dissolved in DMSO (1 mL) and heated at 110° C. in microwave. After the complete conversion of 11, the mixture was diluted with water and extracted with ethyl acetate. The organic layers were combined, dried over anhydrous Na₂SO₄ and concentrated under reduced pressure to give the intermediates 12a-12c. The mixture of 12a-12c (0.2 mmol) and 1-isocyanato-4-methoxybenzene (0.2 mmol) in DCM (1 mL) was stirred at room temperature for 2 h, the solvent was then removed in vacuo to give the crude bromide 13a-13c for next step without further purification.

Finally, the boronic acid pinacol ester (0.3 mmol) and the crude bromide 5 (0.2 mmol) were dissolved in degassed 5:1 dioxane/H₂O. Pd(PPh₃)₄ (0.02 mmol) and 2M solution of K₂CO₃ (0.6 mmol) were added sequentially under Argon and the mixture was heated at 95° C. for 2 h. After cooling to room temperature, the mixture was diluted with water and extracted with ethyl acetate (3×5 mL). The organic layers were combined, dried over anhydrous Na₂SO₄ and concentrated in vacuo. The residue was then purified by preparative HPLC to give the targeted product 7a and 7b as white solid.

In an alternative route, bis-(pinacolato)diboron (0.24 mmol), crude 5, 9, and 13 (0.2 mmol), and PdCl₂(dppf) (0.02 mmol) were dissolved in degassed dioxane (5 mL). After refluxing for 2 h, the mixture was diluted with water and extracted with ethyl acetate (3×5 mL). The organic layers were combined, dried over anhydrous Na₂SO₄ and concentrated in vacuo to give crude boronic acid pinacol ester. Followed the synthesis procedure of 7a, 7c-7k, 10a-10f, 14a-14c were synthesized form crude boronic acid pinacol ester (0.2 mmol) and Ar—Cl (0.2 mmol).

2-Chloroethyl carbonochloridate (1 mmol) was added to a mixture of substituted anilines (1 mmol) and pyridine (3 mmol) in DCM (10 mL) and the reaction was stirred for 24 h. Then, solvent was removed in vacuo and the remaining residue redissolved in water and ethyl acetate. The organic layers were combined, dried over anhydrous Na₂SO₄ and concentrated in vacuo to give crude 15. KOH (10 mmol) was added to the mixture of crude 15 (1 mmol) in EtOH (10 mL). Then the mixture was refluxed until the complete conversion of 15. The solvent was removed in vacuo and the remaining residue was redissolved in water and ethyl acetate. The organic layers were combined, dried over anhydrous Na₂SO₄, concentrated in vacuo, and purified by silica gel to give intermediates 16. The mixture of iodobenzene (0.2 mmol), 2-(Pyrrolidin-1-yl)ethanamine (0.6 mmol), Pd(dba)₂ (0.01 mmol), BINAP (0.01 mmol), and Cs₂CO₃ (0.6 mmol) in dioxane (1 mL) was refluxing for 24 h. After cooling to room temperature, water and ethyl acetate were added. Then the organic layers were combined, dried over anhydrous Na₂SO₄, concentrated in vacuo, and purified by silica gel to give intermediates 17. Then 18a and 18b-18n were synthesized from 17 and 16 respectively followed the synthetic procedure of 10a-10f from 8.

### 3-(3-(4-(7H-pyrrolo[2,3-d]pyrimidin-4-yl)phenyl)ureido)-N-isopropylbenzamide (3)

45% yield in 4 steps. ¹H-NMR (DMSO-d₆, 400 MHz) δ 12.28 (s, br, 1H), 9.06 (s, 1H), 8.96 (s, 1H), 8.81 (s, 1H), 8.18-8.16 (m, 3H), 7.87 (s, 1H), 7.71-7.64 (m, 4H), 7.46-7.44 (m, 1H), 7.38-7.36 (m, 1H), 6.95 (s, 1H), 4.14-4.07 (m, 1H), 1.18 (d, J=5.2 Hz, 6H); ¹³C-NMR (DMSO-d₆, 100 MHz) δ 165.50, 152.70, 152.38, 152.19, 147.81, 143.00, 139.47, 135.77, 130.01, 129.56, 128.51, 127.01, 120.81, 120.71, 118.10, 117.70, 113.78, 101.66, 40.95, 22.27; LC/MS (M+H⁺): 415.11; HRMS (ESI-Orbitrap) Calcd for C₂₃H₂₃N₆O₂: 415.1882 [M+H⁺], Found 415.1872.

### 3-(3-(4-(1H-pyrazol-4-yl)phenyl)ureido)-N-isopropylbenzamide (7a)

68% yield in 3 steps. ¹H-NMR (DMSO-d₆, 400 MHz) δ 12.86 (s, br, 1H), 8.82 (s, 1H), 8.69 (s, 1H), 8.18-8.16 (m, 1H), 7.96-7.93 (m, 1H), 7.84-7.79 (m, 1H), 7.78-7.74 (m, 1H), 7.72-7.68 (m, 1H), 7.65-7.60 (m, 1H), 7.53-7.51 (m, 2H), 7.45-7.41 (m, 3H), 7.36-7.32 (m, 1H), 4.08 (q, J=6.4 Hz, 1H), 1.17 (d, J=6.4 Hz, 6H); HRMS (ESI-Orbitrap) Calcd for C₂₀H₂₂N₅O₂: 364.1773 [M+H⁺], Found 364.1792.

### N-Isopropyl-3-(3-(4-(pyridin-4-yl)phenyl)ureido)benzamide (7b)

65% yield in 3 steps. ¹H-NMR (DMSO-d₆, 400 MHz) δ 9.12 (s, 1H), 9.04 (s, 1H), 8.78 (d, J=5.6 Hz, 2H), 8.19-8.17 (m, 1H), 8.12 (d, J=5.6 Hz, 2H), 7.96 (d, J=8.8 Hz, 2H), 7.89-7.88 (m, 1H), 7.70 (d, J=8.8 Hz, 2H), 7.64-7.61 (m, 1H), 7.46-7.44 (m, 1H), 7.38-7.34 (m, 1H), 4.08 (q, J=6.4 Hz, 1H), 1.17 (d, J=6.4 Hz, 6H); ¹³C-NMR (DMSO-d₆, 100 MHz) δ 165.46, 152.57, 152.35, 144.23, 142.99, 139.46, 135.77, 128.52, 128.49, 127.52, 121.84, 120.82, 120.68, 118.42, 117.74, 40.94, 22.28; LC/MS (M+H⁺): 375.14.

### 3-(3-(4-(2-Aminopyrimidin-4-yl)phenyl)ureido)-N-isopropylbenzamide (7c)

52% yield in 4 steps. ¹H-NMR (DMSO-d₆, 400 MHz) δ 9.43 (s, br, 2H), 8.92-8.80 (m, 1H), 8.32-8.17 (m, 1H), 8.24-8.17 (m, 1H), 8.12-8.10 (m, 2H), 7.90 (s, 1H), 7.66-7.61 (m, 3H), 7.45-7.34 (m, 3H), 7.28 (s, 1H), 4.09 (q, J=6.8 Hz, 1H), 1.16 (d, J=6.8 Hz, 6H); ¹³C-NMR (DMSO-d₆, 100 MHz) δ 166.79, 165.51, 159.06, 152.36, 152.00, 143.87, 139.51, 135.78, 128.67, 128.46, 128.19, 120.74, 120.66, 117.72, 117.64, 105.05, 40.93, 22.27; HRMS (ESI-Orbitrap) Calcd for C₂₁H₂₃N₅O₂: 391.1882 [M+H⁺], Found 391.1889.

### N-Isopropyl-4-[3-[4-(1H-pyrrolo[2,3-b]pyridin-4-yl)-phenyl]-ureido]-benzamide (7d)

40% yield in 4 steps. ¹H-NMR (DMSO-d₆, 400 MHz) δ 12.26 (s, br, 1H), 9.08 (s, 1H), 8.97 (s, 1H), 8.80 (s, 1H), 8.25-8.14 (m, 3H), 8.12 (d, J=8.8 Hz, 2H), 7.87 (s, 1H), 7.76 (d, J=8.8 Hz, 2H), 7.46-6.36 (m, 2H), 6.96-6.95 (m, 2H), 4.08 (m, 1H), 1.17 (d, J=6.4 Hz, 6H); LC/MS (M+H⁺): 414.15.

### 4-[3-[4-(7-Ethyl-8-oxo-8,9-dihydro-7H-purin-6-yl)-phenyl]-ureido]-N-isopropyl-benzamide (7e)

45% yield in 4 steps. ¹H-NMR (DMSO-d₆, 400 MHz) δ 12.08 (s, br, 1H), 9.42 (s, 1H), 8.84-8.82 (m, 1H), 8.76 (s, 1H), 8.35-8.14 (m, 3H), 8.12 (d, J=8.8 Hz, 2H), 7.96 (d, J=8.8 Hz, 2H), 7.69-7.59 (m, 1H), 4.08 (m, 1H), 3.35 (q, J=3.2 Hz, 2H), 1.17 (d, J=6.4 Hz, 6H), 1.07 (t, J=3.2 Hz, 3H); LC/MS (M+H⁺): 460.17.

### N-Isopropyl-4-[3-[4-(9H-purin-6-yl)-phenyl]-ureido]-benzamide (7f)

29% yield in 4 steps. ¹H-NMR (DMSO-d₆, 400 MHz) δ 11.98. (s, br, 1H), 9.48 (s, 1H), 8.81-8.79 (m, 1H), 8.76 (s, 1H), 8.35-8.14 (m, 3H), 8.12 (d, J=8.8 Hz, 2H), 7.96 (d, J=8.8 Hz, 2H), 7.69-7.59 (m, 2H), 6.95 (s, 1H), 4.08 (m, 1H), 1.17 (d, J=6.4 Hz, 6H); HRMS (ESI-Orbitrap) Calcd for C₂₂H₂₂N₇O₂: 416.1835 [M+H⁺], Found 416.1849.

### N-Isopropyl-3-(3-(4-(5-methyl-7H-pyrrolo[2,3-d]pyrimidin-4-yl)phenyl)ureido)benzamide (7g)

40% yield in 4 steps. ¹H-NMR (DMSO-d₆, 400 MHz) δ 12.63 (s, 1H), 9.21 (s, 1H), 9.10 (s, 1H), 8.92 (s, 1H), 8.19-8.18 (m, 1H), 7.89 (s, 1H), 7.74-7.68 (m, 4H), 7.65-7.61 (m, 2H), 7.46-7.44 (m, 1H), 7.39-7.35 (m, 1H), 4.10 (q, J=6.4 Hz, 1H), 2.10 (s, 3H), 1.17 (d, J=6.4 Hz, 6H); ¹³C-NMR (DMSO-d₆, 100 MHz) M65.52, 159.07, 158.74, 153.86, 152.50, 152.09, 145.81, 142.76, 139.59, 135.78, 130.90, 128.46, 125.16, 120.78, 117.71, 117.44, 114.54, 111.64, 40.94, 22.27, 12.51; HRMS (ESI-Orbitrap) Calcd for C₂₄H₂₅N₆O₂: 429.2039 [M+H⁺], Found 429.2029.

### N-Isopropyl-3-(3-(4-(6-methyl-7H-pyrrolo[2,3-d]pyrimidin-4-yl)phenyl)ureido)benzamide (7h)

35% yield in 4 steps. ¹H-NMR (DMSO-d₆, 400 MHz) δ 12.22 (s, br, 1H), 9.07 (s, 1H), 8.96 (s, 1H), 8.74 (s, 1H), 8.20-8.18 (m, 1H), 8.14-8.12 (m, 2H), 7.88 (s, 1H), 7.70-7.68 (m, 2H), 7.66-7.61 (m, 1H), 7.46-7.44 (m, 1H), 7.38-7.34 (m, 1H), 6.69 (s, 1H), 4.10 (q, J=6.8 Hz, 1H), 1.17 (d, J=6.8 Hz, 6H); LC/MS (M+H⁺): 429.17.

### 3-(3-(4-(5,6-Dimethyl-7H-pyrrolo[2,3-d]pyrimidin-4-yl)phenyl)ureido)-N-isopropylbenzamide (7i)

32% yield in 4 steps. ¹H-NMR (DMSO-d₆, 400 MHz) δ 9.04 (s, 1H), 8.99 (s, 1H), 8.74-8.73 (m, 1H), 8.19-8.17 (m, 1H), 7.87 (s, 1H), 7.68-7.61 (m, 5H), 7.46-7.44 (m, 1H), 7.38-7.34 (m, 1H), 4.11 (q, J=6.8 Hz, 1H), 1.97 (s, 3H), 1.17 (d, J=6.8 Hz, 6H); LC/MS (M+H⁺): 443.16.

### 3-(3-(2-(2-(Dimethylamino)ethoxy)-4-(5-methyl-7H-pyrrolo[2,3-d]pyrimidin-4-yl)phenyl)ureido)-N-isopropylbenzamide (71)

46% yield in 4 steps. ¹H-NMR (DMSO-d₆, 400 MHz) δ 12.3 (s, br, 1H), 9.73 (s, 1H), 9.60 (s, 1H), 8.84 (s, 1H), 8.45 (s, 1H), 8.34-8.32 (m, 1H), 8.20-8.18 (m, 1H), 7.90 (s, 1H), 7.71-7.70 (m, 1H), 7.51 (s, 1H), 7.48-7.47 (m, 1H), 7.40-7.33 (m, 2H), 4.51 (t, J=4.6 Hz, 2H), 4.10 (q, J=6.4 Hz, 1H), 3.63 (t, J=4.6 Hz, 2H), 2.94 (s, 6H), 2.12 (s, 3H), 1.17 (d, J=6.4 Hz, 6H); ¹³C-NMR (DMSO-d₆, 100 MHz) δ 165.44, 158.91, 158.58, 154.45, 152.33, 152.23, 146.68, 145.76, 139.52, 135.78, 131.26, 128.52, 127.95, 123.80, 120.68, 118.34, 117.72, 114.64, 113.18, 111.10, 62.96, 55.44, 42.65, 40.94, 22.26, 12.78; LC/MS (M+H⁺): 516.13.

### N-Isopropyl-3-(3-(4-(5-methyl-7H-pyrrolo[2,3-d]pyrimidin-4-yl)-2-(trifluoromethyl)phenyl)ureido)-benzamide (7j)

47% yield in 4 steps. ¹H-NMR (DMSO-d₆, 400 MHz) δ 12.19 (s, br, 1H), 9.71 (s, 1H), 8.83 (s, 1H), 8.33-8.26 (m, 2H), 8.21-8.19 (m, 1H), 8.01-8.00 (m, 2H), 7.85 (s, 1H), 7.70-7.68 (m, 1H), 7.48-7.46 (m, 2H), 7.41-7.37 (m, 1H), 4.10 (q, J=6.8 Hz, 1H), 2.09 (s, 3H), 1.16 (d, J=6.8 Hz, 6H); ¹³C-NMR (DMSO-d₆, 100 MHz) δ 165.37, 155.31, 152.57, 152.17, 149.46, 139.24, 137.50, 135.83, 134.08, 131.87, 128.70, 127.18, 126.48, 125.15, 124.24, 122.44, 120.96, 120.58, 117.53, 114.95, 108.93, 40.93, 22.27, 12.81; HRMS (ESI-Orbitrap), Calcd for C₂₅H₂₄F₃N₆O₂: 497.1913 [M+H⁺], Found 497.1902.

### 3-(3-(2-Fluoro-4-(5-methyl-7H-pyrrolo[2,3-d]pyrimidin-4-yl)phenyl)ureido)-N-isopropylbenzamide (7k)

42% yield in 4 steps. ¹H-NMR (DMSO-d₆, 400 MHz) δ 12.20 (s, br, 1H), 9.46 (s, 1H), 8.89 (s, 1H), 8.81 (s, 1H), 8.52-8.49 (m, 1H), 8.25-8.19 (m, 1H), 7.88-7.84 (m, 1H), 7.68-7.64 (m, 2H), 7.52-7.48 (m, 3H), 7.42-7.39 (m, 1H), 4.10 (q, J=6.8 Hz, 1H), 2.10 (s, 3H), 1.17 (d, J=6.8 Hz, 6H); ¹³C-NMR (DMSO-d₆, 100 MHz) δ 165.44, 154.88, 152.40, 152.10, 149.96, 148.37, 139.27, 135.83, 129.21, 128.61, 126.88, 126.43, 120.84, 120.56, 119.50, 117.45, 116.19, 115.99, 114.82, 109.82, 40.95, 22.26, 12.75; HRMS (ESI-Orbitrap), Calcd for C₂₄H₂₄FN₆O₂: 447.1945 [M+H⁺], Found 447.1934.

### 1-(4-(5-Methyl-7H-pyrrolo[2,3-d]pyrimidin-4-yl)phenyl)-3-phenylurea (10a)

62% yield in 3 steps. ¹H-NMR (DMSO-d₆, 400 MHz) δ 12.56 (s, br, 1H), 9.18 (s, 1H), 8.94-8.90 (m, 2H), 7.78-7.67 (m, 4H), 7.62-7.58 (m, 1H), 7.50-7.48 (m, 2H), 7.32-7.28 (m, 2H), 7.01-6.98 (m, 1H), 2.10 (s, 3H); HRMS, Calcd for C₂₀H₁₈N₅O: 344.1511 [M+H⁺], Found 344.1526.

### 1-(3-Fluorophenyl)-3-(4-(5-methyl-7H-pyrrolo[2,3-d]pyrimidin-4-yl)phenyl)urea (10b)

51% yield in 3 steps. ¹H-NMR (DMSO-d₆, 400 MHz) δ 12.55 (s, br, 1H), 9.17-9.12 (m, 2H), 8.88 (s, 1H), 7.69-7.68 (m, 4H), 7.60-7.50 (m, 2H), 7.36-7.31 (m, 1H), 7.18-7.16 (m, 1H), 6.84-6.79 (m, 1H), 2.10 (s, 3H); LC/MS (M+H⁺): 362.11.

### 1-(2-Methoxyphenyl)-3-(4-(5-methyl-7H-pyrrolo[2,3-d]pyrimidin-4-yl)phenyl)urea (10c)

60% yield in 3 steps. ¹H-NMR (DMSO-d₆, 400 MHz) δ 12.49 (s, 1H), 9.66 (s, 1H), 8.88 (s, 1H), 8.35 (s, 1H), 8.16-8.14 (m, 1H), 7.69-7.68 (m, 4H), 7.56 (s, 1H), 7.06-7.03 (m, 1H), 7.00-6.98 (m, 1H), 6.96-6.89 (m, 1H), 3.90 (s, 3H), 2.10 (s, 3H); ¹³C-NMR (DMSO-d₆, 100 MHz) δ 154.87, 152.24, 152.18, 147.80, 146.90, 142.24, 130.81, 128.35, 127.60, 126.70, 122.12, 120.52, 118.45, 117.12, 114.61, 110.93, 110.77, 55.75, 12.66; LC/MS (M+H⁺): 374.09.

### 1-(3-Methoxyphenyl)-3-(4-(5-methyl-7H-pyrrolo[2,3-d]pyrimidin-4-yl)phenyl)urea (10d)

57% yield in 3 steps. ¹H-NMR (DMSO-d₆, 400 MHz) δ 12.61 (s, br, 1H), 9.14 (s, 1H), 8.93-8.91 (m, 2H), 7.72-7.67 (m, 4H), 7.60 (s, 1H), 7.23-7.18 (m, 2H), 6.98-6.96 (m, 1H), 6.59-6.57 (m, 1H), 3.74 (s, 3H), 2.10 (s, 3H); ¹³C-NMR (DMSO-d₆, 100 MHz) δ 159.64, 153.71, 152.42, 152.07, 145.64, 142.89, 140.85, 130.91, 129.49, 128.52, 124.83, 117.37, 114.51, 111.75, 110.63, 107.32, 104.13, 54.88, 12.50; LC/MS (M+H⁺): 374.09.

### 1-(4-Methoxyphenyl)-3-(4-(5-methyl-7H-pyrrolo[2,3-d]pyrimidin-4-yl)phenyl)urea (10e)

45% yield in 3 steps. ¹H-NMR (DMSO-d₆, 400 MHz) δ 11.89 (s, br, 1H), 8.86 (s, 1H), 8.71 (s, 1H), 8.60 (s, 1H), 7.61-7.60 (m, 4H), 7.38 (d, J=8.8 Hz, 2H), 7.35 (s, 1H), 6.88 (d, J=8.8 Hz, 2H), 3.73 (s, 3H), 2.10 (s, 3H); LC/MS (M+H⁺): 374.14; HRMS (ESI-Orbitrap) Calcd for C₂₁H₂₀N₅O₂: 374.1617 [M+H⁺], Found 374.1608.

### 1-[4-(5-Methyl-7H-pyrrolo[2,3-d]pyrimidin-4-yl)-phenyl]-3-thiazol-2-yl-urea (10f)

40% yield in 3 steps. ¹H-NMR (DMSO-d₆, 400 MHz) δ 11.95 (s, br, 1H), 8.76 (s, 1H), 8.71 (s, 1H), 8.60 (s, 1H), 7.71-7.68 (m, 4H), 7.53-7.48 (m, 1H), 6.99-6.97 (m, 1H), 6.58-6.56 (m, 1H), 2.10 (s, 3H); LC/MS (M+H⁺): 351.14.

### 1-[4-(5-Methyl-7H-pyrrolo[2,3-d]pyrimidin-4-yl)-phenyl]-3-pyridin-2-yl-urea (10g)

48% yield in 3 steps. LC/MS (M+H⁺): 345.12.

### 3-(4-Methoxyphenyl)-1-methyl-1-(4-(5-methyl-7H-pyrrolo[2,3-d]pyrimidin-4-yl)phenyl)urea (14a)

55% yield in 3 steps. ¹H-NMR (DMSO-d₆, 400 MHz) δ 12.40 (s, br, 1H), 8.87 (s, 1H), 8.40 (s, 1H), 7.73 (d, J=8.8 Hz, 2H), 7.55-7.54 (m, 1H), 7.52 (d, J=8.8 Hz, 2H), 7.36 (d, J=6.8 Hz, 2H), 6.85 (d, J=6.8 Hz, 2H), 3.38 (s, 3H), 3.17 (s, 3H), 2.11 (s, 3H); ¹³C-NMR (DMSO-d₆, 100 MHz) δ 159.21, 154.89, 152.28, 147.29, 146.42, 134.02, 132.78, 130.47, 127.57, 127.29, 124.54, 122.05, 114.80, 113.53, 110.66, 55.12, 37.10, 12.67; LC/MS (M+H⁺): 388.18; HRMS (ESI-Orbitrap) Calcd for C₂₂H₂₂N₅O₂: 388.1773 [M+H⁺], Found 388.1764.

### 3-(4-Methoxyphenyl)-1-(4-(5-methyl-7H-pyrrolo[2,3-d]pyrimidin-4-yl)phenyl)-1-(2-(pyrrolidin-1-yl)ethyl)urea (14b)

18% yield in 5 steps. ¹H-NMR (DMSO-d₆, 400 MHz) δ 9.59 (s, br, 1H), 8.79 (s, 1H), 8.12 (s, 1H), 7.80 (d, J=8.4 Hz, 2H), 7.57 (d, J=8.4 Hz, 2H), 7.43 (s, 1H), 7.32 (d, J=6.8 Hz, 2H), 6.82 (d, J=6.8 Hz, 2H), 4.08-4.04 (m, 2H), 3.70 (s, 3H), 3.65-3.64 (m, 2H), 3.37-3.32 (m, 2H), 3.12-3.06 (m, 2H), 2.14 (s, 3H), 2.04-2.02 (m, 2H), 1.90-1.86 (m, 2H); HRMS (ESI-Orbitrap), Calcd for C₂₇H₃₁N₆O₂: 471.2508 [M+H⁺], Found 471.2516.

### 3-(4-Methoxyphenyl)-1-(4-(5-methyl-7H-pyrrolo[2,3-d]pyrimidin-4-yl)phenyl)-1-(2-(piperidin-1-yl)ethyl)urea (14c)

19% yield in 5 steps. ¹H-NMR (DMSO-d₆, 400 MHz) δ 12.02 (s, br, 1H), 8.77 (s, 1H), 8.15 (s, 1H), 7.80 (d, J=8.4 Hz, 2H), 7.56 (d, J=8.4 Hz, 2H), 7.41 (s, 1H), 7.31 (d, J=8.8 Hz, 2H), 6.83 (d, J=8.8 Hz, 2H), 4.09-4.06 (m, 2H), 3.70 (s, 3H), 3.56-3.54 (m, 2H), 3.28-3.24 (m, 2H), 2.97-2.91 (m, 2H), 2.14 (s, 3H), 1.85-1.81 (m, 2H), 1.68-1.62 (m, 4H); LC/MS (M+H⁺): 485.15.

### 3-(4-(5-Methyl-7H-pyrrolo[2,3-d]pyrimidin-4-yl)phenyl)-1-phenyl-1-(2-(pyrrolidin-1-yl)ethyl)urea (18a)

46% yield in 4 steps. ¹H NMR (DMSO-d₆, 400 MHz) δ 12.04 (s, br, 1H), 9.57 (s, 1H), 8.74 (s, 1H), 8.20 (s, 1H), 7.63-7.55 (m, 4H), 7.53-7.51 (m, 2H), 7.48-7.46 (m, 1H), 7.42-7.39 (m, 2H), 4.04-4.00 (m, 2H), 3.68-3.65 (m, 2H), 3.31-3.29 (m, 2H), 3.08-3.07 (m, 2H), 2.04-2.02 (m, 5H), 1.90-1.87 (m, 2H); LC/MS (M+H⁺): 441.00.

### 1-(2-Hydroxyethyl)-3-(4-(5-methyl-7H-pyrrolo[2,3-d]pyrimidin-4-yl)phenyl)-1-phenylurea (18b)

From commercially available 2-phenylamino-ethanol, 18b was synthesized in 62% yield through 3 steps. ¹H-NMR (DMSO-d₆, 400 MHz) δ 8.83-8.81 (m, 1H), 8.40 (s, 1H), 7.64-7.58 (m, 4H), 7.47-7.43 (m, 3H), 7.39-7.37 (m, 2H), 7.32-7.29 (m, 1H), 3.76 (t, J=6.4 Hz, 2H), 3.56 (t, J=6.4 Hz, 2H), 2.06 (s, 3H); ¹³C-NMR (DMSO-d₆, 100 MHz) δ 154.43, 152.14, 146.62, 142.62, 130.30, 129.37, 129.09, 127.78, 127.66, 127.59, 126.52, 119.18, 118.67, 114.56, 111.08, 58.79, 52.24, 12.61; HRMS (ESI-Orbitrap), Calcd for C₂₂H₂₂N₅O₂: 388.1773 [M+H⁺], Found 388.1764.

### 1-(2-Fluorophenyl)-1-(2-hydroxyethyl)-3-(4-(5-methyl-7H-pyrrolo[2,3-d]pyrimidin-4-yl)phenyl)urea (18c)

49% yield in 5 steps. ¹H-NMR (DMSO-d₆, 400 MHz) δ 12.59 (s, br, 1H), 8.89 (s, 1H), 8.75 (s, 1H), 7.66-7.54 (m, 4H), 7.52-7.50 (m, 1H), 7.42-7.40 (m, 1H), 7.39-7.37 (m, 1H), 7.34-7.26 (m, 2H), 3.72 (t, J=6.4 Hz, 2H), 3.57 (t, J=6.4 Hz, 2H), 2.07 (s, 3H); ¹³C-NMR (DMSO-d₆, 100 MHz) δ 159.17, 158.37, 156.71, 154.39, 153.62, 152.07, 145.55, 143.00, 130.74, 130.49, 129.81, 128.64, 125.11, 119.20, 116.55, 114.51, 111.76, 58.92, 51.98, 12.47; LC/MS (M+H⁺): 406.07.

### 1-(3-Fluorophenyl)-1-(2-hydroxyethyl)-3-(4-(5-methyl-7H-pyrrolo[2,3-d]pyrimidin-4-yl)phenyl)urea (18d)

45% yield in 5 steps. ¹H-NMR (DMSO-d₆, 400 MHz) δ 12.54 (s, br, 1H), 8.88 (s, 1H), 8.66 (s, 1H), 7.68-7.62 (m, 4H), 7.57 (s, 1H), 7.48-7.43 (m, 1H), 7.32-7.29 (m, 1H), 7.23-7.21 (m, 1H), 7.15-7.10 (m, 1H), 3.79 (t, J=6.0 Hz, 2H), 3.58 (t, J=6.0 Hz, 2H), 2.07 (s, 3H); ¹³C-NMR (DMSO-d₆, 100 MHz) δ 163.53, 161.11, 154.21, 152.14, 146.34, 144.40, 142.65, 130.67, 130.58, 130.36, 128.04, 123.44, 118.83, 114.75, 114.52, 112.97, 111.26, 58.81, 52.25, 12.58; HRMS (ESI-Orbitrap), Calcd for C₂₂H₂₁FN₅O₂: 406.1679 [M+H⁺], Found 406.1686.

### 1-(4-Fluorophenyl)-1-(2-hydroxyethyl)-3-(4-(5-methyl-7H-pyrrolo[2,3-d]pyrimidin-4-yl)phenyl)urea (18e)

41% yield in 5 steps. ¹H-NMR (DMSO-d₆, 400 MHz) δ 12.51 (s, br, 1H), 8.86 (s, 1H), 8.37 (s, 1H), 7.66-7.62 (m, 4H), 7.60 (s, 1H), 7.44-7.41 (m, 2H), 7.29-7.25 (m, 2H), 3.73 (t, J=6.0 Hz, 2H), 3.55 (t, J=6.0 Hz, 2H), 2.06 (s, 3H); ¹³C-NMR (DMSO-d₆, 100 MHz) δ 161.76, 159.34, 154.39, 152.13, 146.35, 142.72, 138.65, 130.30, 130.14, 128.01, 118.83, 116.20, 115.98, 114.55, 111.24, 58.70, 52.33, 12.57; LC/MS (M+H⁺): 406.06; HRMS (ESI-Orbitrap), Calcd for C₂₂H₂₁FN₅O₂: 406.1679 [M+H⁺], Found 406.1670.

### 1-(2-Chlorophenyl)-1-(2-hydroxyethyl)-3-(4-(5-methyl-7H-pyrrolo[2,3-d]pyrimidin-4-yl)phenyl)urea (18f)

40% yield in 5 steps. ¹H-NMR (DMSO-d₆, 400 MHz) δ 12.70 (s, br, 1H), 8.92 (s, 1H), 8.72 (s, 1H), 7.69-7.63 (m, 5H), 7.53-7.52 (m, 1H), 7.47-7.43 (m, 1H), 7.36-7.33 (m, 1H), 3.78 (t, J=6.0 Hz, 2H), 3.57 (t, J=6.0 Hz, 2H), 2.07 (s, 3H); ¹³C-NMR (DMSO-d₆, 100 MHz) δ 158.42, 158.07, 154.22, 152.08, 145.63, 144.25, 142.95, 133.22, 130.69, 130.46, 128.58, 127.53, 126.23, 126.19, 118.86, 114.51, 111.70, 58.82, 52.31, 12.49; HRMS (ESI-Orbitrap), Calcd for C₂₂H₂₁ClN₅O₂: 422.1384 [M+H⁺], Found 422.1369.

### 1-(3-Chlorophenyl)-1-(2-hydroxyethyl)-3-(4-(5-methyl-7H-pyrrolo[2,3-d]pyrimidin-4-yl)phenyl)urea (18g)

45% yield in 5 steps. ¹H-NMR (DMSO-d₆, 400 MHz) δ 8.85 (s, 1H), 7.64-7.58 (m, 5H), 7.57-7.56 (m, 1H), 7.52 (s, 1H), 7.47-7.45 (m, 1H), 7.44-7.39 (m, 2H), 3.58 (t, J=6.0 Hz, 2H), 3.42 (t, J=6.0 Hz, 2H), 2.06 (s, 3H); ¹³C-NMR (DMSO-d₆, 100 MHz) δ 158.28, 157.95, 154.22, 152.12, 146.21, 142.75, 139.25, 132.59, 131.76, 130.35, 130.24, 129.21, 128.36, 128.15, 118.99, 114.53, 111.33, 58.79, 51.67, 12.58; LC/MS (M+H⁺): 422.06.

### 1-(4-Chlorophenyl)-1-(2-hydroxyethyl)-3-(4-(5-methyl-7H-pyrrolo[2,3-d]pyrimidin-4-yl)phenyl)urea (18h)

48% yield in 5 steps. ¹H-NMR (DMSO-d₆, 400 MHz) δ 12.49 (s, br, 1H), 8.87 (s, 1H), 8.56 (s, 1H), 7.66-7.60 (m, 4H), 7.55 (s, 1H), 7.50-7.47 (m, 2H), 7.43-7.40 (m, 2H), 3.75 (t, J=6.0 Hz, 2H), 3.56 (t, J=6.0 Hz, 2H), 2.06 (s, 3H); HRMS (ESI-Orbitrap), Calcd for C₂₂H₂₁ClN₅O₂: 422.1384 [M+H⁺], Found 422.1375.

### 1-(2-Hydroxyethyl)-3-(4-(5-methyl-7H-pyrrolo[2,3-d]pyrimidin-4-yl)phenyl)-1-o-tolylurea (181)

45% yield in 5 steps. ¹H-NMR (DMSO-d₆, 400 MHz) δ 12.56 (s, br, 1H), 8.88 (s, 1H), 8.18 (s, 1H), 7.65-7.58 (m, 5H), 7.36-7.29 (m, 4H), 3.74 (t, J=6.0 Hz, 2H), 3.58 (t, J=6.0 Hz, 2H), 2.22 (s, 3H), 2.06 (s, 3H); ¹³C-NMR (DMSO-d₆, 100 MHz) δ 158.40, 158.06, 154.37, 153.84, 152.08, 145.75, 143.02, 136.21, 131.13, 130.43, 129.30, 128.47, 127.64, 127.09, 118.82, 114.50, 111.62, 58.82, 51.71, 17.31, 12.51; LC/MS (M+H⁺): 402.09.

### 1-(2-Hydroxyethyl)-3-(4-(5-methyl-7H-pyrrolo[2,3-d]pyrimidin-4-yl)phenyl)-1-m-tolylurea (18j)

43% yield in 5 steps. ¹H-NMR (DMSO-d₆, 400 MHz) δ 12.70 (s, br, 1H), 8.92 (s, 1H), 8.40 (s, 1H), 7.68-7.61 (m, 5H), 7.35-7.31 (m, 1H), 7.21 (s, 1H), 7.16-7.12 (m, 2H), 3.74 (t, J=6.4 Hz, 2H), 3.53 (t, J=6.4 Hz, 2H), 2.35 (s, 3H), 2.07 (s, 3H); ¹³C-NMR (DMSO-d₆, 100 MHz) δ 158.11, 154.38, 153.97, 152.09, 145.87, 142.96, 142.34, 138.77, 130.42, 129.16, 128.37, 128.20, 127.32, 124.73, 118.69, 114.50, 111.55, 58.75, 52.20, 20.95, 12.53; LC/MS (M+H⁺): 402.06.

### 1-(2-Hydroxyethyl)-3-(4-(5-methyl-7H-pyrrolo[2,3-d]pyrimidin-4-yl)phenyl)-1-p-tolylurea (18k)

39% yield in 5 steps. ¹H-NMR (DMSO-d₆, 400 MHz) δ 12.60 (s, br, 1H), 8.89 (s, 1H), 8.28 (s, 1H), 7.66-7.62 (m, 4H), 7.60-7.58 (m, 1H), 7.26-7.25 (m, 4H), 3.72 (t, J=6.0 Hz, 2H), 3.54 (t, J=6.0 Hz, 2H), 2.34 (s, 3H), 2.07 (s, 3H); LC/MS (M+H⁺): 402.09; HRMS (ESI-Orbitrap) Calcd for C₂₃H₂₄N₅O₂: 402.1930 [M+H⁺], Found 402.1920.

### 1-(2-Hydroxyethyl)-1-(2-methoxyphenyl)-3-(4-(5-methyl-7H-pyrrolo[2,3-d]pyrimidin-4-yl)phenyl)urea (181)

47% yield in 5 steps. ¹H-NMR (DMSO-d₆, 400 MHz) δ 8.83 (s, 1H), 7.59-7.58 (m, 5H), 7.49-7.48 (m, 1H), 7.38-7.32 (m, 2H), 7.16-7.14 (m, 1H), 7.04-7.00 (m, 1H), 3.80 (s, 3H), 3.62-3.51 (m, 4H), 2.06 (s, 3H); ¹³C-NMR (DMSO-d₆, 100 MHz) δ 158.30, 155.25, 154.72, 154.23, 152.10, 146.10, 143.02, 130.49, 130.32, 130.01, 128.97, 128.16, 120.86, 118.67, 114.51, 112.74, 111.39, 58.74, 55.67, 51.22, 12.54; LC/MS (M+H⁺): 418.07.

### 1-(2-Hydroxyethyl)-1-(3-methoxyphenyl)-3-(4-(5-methyl-7H-pyrrolo[2,3-d]pyrimidin-4-yl)phenyl)urea (18m)

46% yield in 5 steps. ¹H-NMR (DMSO-d₆, 400 MHz) δ 12.50 (s, br, 1H), 8.86 (s, 1H), 8.39 (s, 1H), 7.66-7.60 (m, 4H), 7.55 (s, 1H), 7.36-7.32 (m, 1H), 6.98-6.97 (m, 1H), 6.93-6.88 (m, 2H), 3.78 (s, 3H), 3.75 (t, J=6.0 Hz, 2H), 3.56 (t, J=6.0 Hz, 2H), 2.07 (s, 3H); ¹³C-NMR (DMSO-d₆, 100 MHz) δ 159.95, 158.32, 158.10, 154.26, 152.11, 146.22, 143.55, 142.78, 130.36, 130.05, 128.11, 119.77, 118.71, 114.53, 113.57, 112.22, 111.33, 58.75, 55.18, 52.19, 12.57; LC/MS (M+H⁺): 418.05.

### 1-(2-hydroxyethyl)-1-(4-methoxyphenyl)-3-(4-(5-methyl-7H-pyrrolo[2,3-d]pyrimidin-4-yl)phenyl)urea (18n)

48% yield in 5 steps. ¹H-NMR (DMSO-d₆, 400 MHz) δ 12.56 (s, br, 1H), 8.88 (s, 1H), 8.13 (s, 1H), 7.66-7.59 (m, 5H), 7.30 (d, J=6.8 Hz, 2H), 7.01 (d, J=6.8 Hz, 2H), 3.80 (s, 3H), 3.69 (t, J=6.4 Hz, 2H), 3.53 (t, J=6.4 Hz, 2H), 2.06 (s, 3H); HRMS (ESI-Orbitrap), Calcd for C₂₃H₂₄N₅O₃: 418.1879 [M+H⁺], Found 418.1686.

### 1-(2-Aminoethyl)-1-(4-methoxyphenyl)-3-(4-(5-methyl-7H-pyrrolo[2,3-d]pyrimidin-4-yl)phenyl)urea (18o)

52% yield in 4 steps. ¹H-NMR (DMSO-d₆, 400 MHz) δ 12.64 (s, 1H), 8.86 (s, 1H), 8.35 (s, 1H), 7.82 (s, 2H), 7.59 (dt, J=13.0, 6.5 Hz, 5H), 7.53-7.43 (m, 4H), 3.85 (t, J=6.2 Hz, 2H), 2.93-2.82 (m, 2H), 2.00 (dd, J=8.4, 2.5 Hz, 3H). LC/MS (M+H⁺): 415.11.

### 1-(2-Aminoethyl)-1-(4-chlorophenyl)-3-(4-(5-methyl-7H-pyrrolo[2,3-d]pyrimidin-4-yl)phenyl)urea (18p)

45% yield in 4 steps. ¹H-NMR (DMSO-d₆, 400 MHz) δ 12.60 (s, 1H), 8.89 (s, 1H), 8.06 (s, 1H), 7.87 (s, 2H), 7.69 (d, J=8.8 Hz, 2H), 7.62 (d, J=8.8 Hz, 2H), 7.58 (s, 1H), 7.45-7.39 (m, 2H), 7.10-7.04 (m, 2H), 3.87 (t, J=6.2 Hz, 2H), 3.81 (d, J=9.0 Hz, 3H), 2.99-2.88 (m, 2H), 2.06 (d, J=0.9 Hz, 3H). HRMS (ESI-Orbitrap), Calcd for C₂₂H₂₂ClN₅O: 421.1544 [M+H⁺], Found 421.1563.

### 1-(2-(Dimethylamino)ethyl)-1-(4-methoxyphenyl)-3-(4-(5-methyl-7H-pyrrolo[2,3-d]pyrimidin-4-yl)phenyl)urea (18q)

50% yield in 4 steps. ¹H-NMR (DMSO-d₆, 400 MHz) δ 12.55 (s, 1H), 9.42 (s, 1H), 8.88 (s, 1H), 8.10 (s, 1H), 7.69 (d, J=8.8 Hz, 2H), 7.62 (d, J=8.8 Hz, 2H), 7.57 (s, 1H), 7.45-7.38 (m, 2H), 7.11-7.03 (m, 2H), 3.98 (t, J=6.2 Hz, 2H), 3.82 (s, 3H), 3.20 (d, J=5.2 Hz, 2H), 2.88 (t, J=8.5 Hz, 6H), 2.05 (d, J=0.9 Hz, 3H). LC/MS (ESI-Orbitrap), Found 445.21.

### 1-(4-Chlorophenyl)-1-(2-(dimethylamino)ethyl)-3-(4-(5-methyl-7H-pyrrolo[2,3-d]pyrimidin-4-yl)phenyl)urea (18r)

65% yield in 4 steps. ¹H-NMR (DMSO-d₆, 400 MHz) δ 12.70 (s, 1H), 9.52 (s, 1H), 8.93 (s, 1H), 8.48 (s, OH), 7.67 (dd, J=23.4, 8.8 Hz, 4H), 7.62-7.49 (m, 5H), 4.04 (t, J=6.2 Hz, 2H), 3.21 (d, J=4.7 Hz, 2H), 2.89 (d, J=3.5 Hz, 6H), 2.06 (d, J=0.7 Hz, 3H). LC/MS (ESI-Orbitrap), Found 449.21.

### 1-(4-Chlorophenyl)-3-(4-(5,6-dimethyl-7H-pyrrolo[2,3-d]pyrimidin-4-yl)-2-fluorophenyl)-1-(2-hydroxyethyl)urea (18s)

52% yield in 4 steps. ¹H-NMR (DMSO-d₆, 400 MHz) δ 12.67 (s, 1H), 8.84 (s, 1H), 8.54 (s, 1H), 7.93 (t, J=8.3 Hz, 1H), 7.58 (dd, J=11.4, 1.9 Hz, 2H), 7.54-7.40 (m, 4H), 3.80-3.76 (m, 4H), 2.40 (d, J=10.5 Hz, 3H), 1.93 (s, 3H). HRMS (ESI-Orbitrap) Calcd for C₂₃H₂₁ClFN₅O₂: 454.1446 [M+H⁺], Found 454.1434.

### 1-(2-Amino-ethyl)-1-(4-chloro-phenyl)-3-[4-(5,6-dimethyl-7H-pyrrolo[2,3-d]pyrimidin-4-yl)-2-fluoro-phenyl]-urea (18t)

52% yield in 4 steps. ¹H-NMR (DMSO-d₆, 400 MHz) δ 12.30 (s, 1H), 8.76 (s, 1H), 7.85 (d, J=9.0 Hz, 3H), 7.80 (d, J=8.6 Hz, 2H), 7.63-7.56 (m, 4H), 7.51 (dd, J=11.4, 1.8 Hz, 1H), 7.45 (dd, J=8.3, 1.7 Hz, 1H), 3.91 (t, J=6.3 Hz, 2H), 3.10 (qd, J=7.3, 4.8 Hz, 3H), 2.95 (dd, J=11.9, 6.0 Hz, 2H), 2.37 (s, 3H), 1.92 (d, J=4.5 Hz, 3H), 1.18 (t, J=7.3 Hz, 4H). LC/MS (M+H⁺): 453.14.

### 1-(4-Chlorophenyl)-3-(4-(5,6-dimethyl-7H-pyrrolo[2,3-d]pyrimidin-4-yl)-2-fluorophenyl)-1-(2-(dimethylamino)ethyl)urea (18w)

54% yield in 4 steps. ¹H-NMR (DMSO-d₆, 400 MHz) δ 12.38 (s, 1H), 9.33 (s, 1H), 8.76 (s, 1H), 7.86 (s, 1H), 7.78 (t, J=8.2 Hz, 1H), 7.66-7.57 (m, 2H), 7.57-7.42 (m, 3H), 4.02 (t, J=6.3 Hz, 2H), 3.21 (s, 2H), 2.86 (s, 6H), 2.36 (s, 3H), 1.91 (s, 3H). HRMS (ESI-Orbitrap), Calcd for C₂₅H₂₆ClFN₆O: 481.1919 [M+H⁺], Found 481.1909.

### 1-(4-Chlorophenyl)-3-(4-(5,6-dimethyl-7H-pyrrolo[2,3-d]pyrimidin-4-yl)-2-fluorophenyl)-1-(2-methoxyethyl)urea (18x)

50% yield in 4 steps. ¹H-NMR (400 MHz, DMSO) δ 12.63 (s, 1H), 8.83 (s, 1H), 8.54 (s, 1H), 7.86 (t, J=8.2 Hz, 1H), 7.68-7.29 (m, 6H), 3.94-3.84 (m, 4H), 3.66 (s, 3H), 2.39 (s, 3H), 1.93 (s, 3H). LC/MS (ESI-Orbitrap), Found 468.14.

### Synthesis of R10015—

Commercially available reagents and anhydrous solvents were used without further purification unless otherwise specified. Thin layer chromatography (TLC) analyses were performed with precolated silica gel 60 F254. The mass spectra were recorded by LC/MS with Finnigan LCQ Advantage MAX spectrometer of Thermo Electron®. Flash chromatography was performed on prepacked columns of silica gel (230-400 Mesh, 40-63 μm) by CombiFlash® with EtOAc/hexane or MeOH/DCM as eluent. The preparative HPLC was performed on SunFire C18 OBD 10 μm (30×250 mm) with CH3CN+50% MeOH/H2O+0.1% TFA as eluent to purify the targeted compounds. Analytic HPLC was performed on Agilent technologies 1200 series with CH3CN (Solvent B)/H2O+0.9% CH3CN+0.1% TFA (Solvent A) as eluent and the targeted products were detected by UV in the detection range of 215-310 nm. NMR spectra were recorded with a Bruker® 400 MHz spectrometer at ambient temperature with the residual solvent peaks as internal standards. The line positions of multiplets were given in ppm (δ) and the coupling constants (J) were given in Hertz. The high-resolution mass spectra (HRMS, electrospray ionization) experiments were performed with Thermo Finnigan orbitrap mass analyzer. Data were acquired in the positive ion mode at resolving power of 100000 at nth 400. Calibration was performed with an external calibration mixture immediately prior to analysis.

### General Synthetic Procedures—

The Scheme and synthetic procedures described below are for inhibitor R10015. The synthesis of other LIMK inhibitors listed in Table 9 followed a similar protocol.

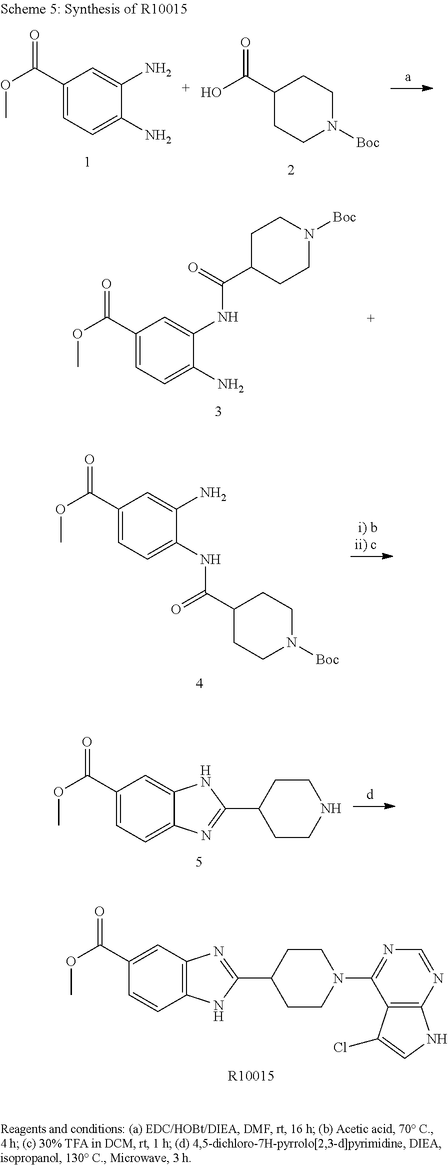

EDC (1-Ethyl-3-(3-dimethylaminopropyl)carbodiimide hydrochloride) (1.2 equiv) was added to stirring mixture of 1 (1 equiv), 2 (1.05 equiv), HOBt (1-hydroxybenzotriazole) (1 equiv), and DIEA (diisopropylethylamine) (3 equiv) in DMF (dimethylformamide). The stirring was continued at rt overnight at which LC-MS demonstrated a complete reaction. The solvent was removed in vacuo to residue which was suspended in EtOAc (ethyl acetate). The suspension was washed by brine and saturated NaHCO₃, dried over anhydrous Na₂SO₄, and evaporated under reduced pressure to give a mixture of crude amide products 3 and 4. Without further purification, this mixture was suspended in acetic acid, and heated at 70° C. for 4 h for ring closure to give the Boc protected 4-yl-piperidinobenzimidazole, which was purified by flash chromatography. The Boc protection was then removed by 30% TFA in DCM to yield 5 as an oil stuff Finally, a mixture of 5 and 4,5-dichloro-7H-pyrrolo[2,3-d]pyrimidine in a small amount of isopropanol was heated at 130° C. under Microwave condition for 3 h to furnish the Limk inhibitor R10015, which was purified by reverse-phase HPLC to give a purity of >95% based on analytical HPLC analysis (UV detection at 254 nm).

### Methyl 2-(1-(5-chloro-7H-pyrrolo[2,3-d]pyrimidin-4-yl)piperidin-4-yl)-1Hbenzo[d]imidazole-5-carboxylate (R10015)

25% yield in 4 steps after HPLC purification. ¹H NMR (DMSO-d6, 400 MHz) δ 12.39 (br, 1H), 8.34 (s, 1H), 8.28 (s, 1H), 8.27 (dd, J=1.2, 8.4 Hz, 1H), 7.87 (d, J=8.8 Hz, 1H), 7.59 (d, J=2.4 Hz, 1H), 4.31-4.47 (m, 2H), 3.91 (s, 3H), 3.51-3.58 (m, 1H), 3.29 (t, J=12.0 Hz, 2H), 2.25-2.34 (m, 2H), 2.11-2.18 (m, 2H); Analytical HPLC purity: single peak observed at UV=254 nm; HRMS (ESI-Orbitrap) Calcd for C20H20ClN6O2: 411.1336 [M+H+], Found 411.1328.

### R10015 Docking Studies:

Inhibitor R10015 was prepared for glide docking using LigPrep (Schrodinger, LLC, NY). The chain A of PDB ID 3S95 was prepared using protein preparation wizard in Maestro V 9.8 (Schrodinger, LLC, NY) by removing water molecules and bound ligand, and adding hydrogen atoms. The docking grid was generated around the original ligand with a box size of 18×18×18 Å3. Docking was conducted without any constraint. The top scored docking pose was merged to the protein for energy minimization using Prime (Schrodinger, LLC, NY).

### LIMK1 Biochemical Assay:

Biochemical LIMK1 assays for all inhibitors were carried out in Reaction Biology Corporation (http://www.reactionbiology.com) and followed the protocols described on its website. Compounds were tested in a 10-dose IC₅₀ mode with 3-fold series dilution starting at 10 μM. Control compound Staurosporine was tested in 10-dose IC₅₀ mode with 3-fold serial dilution starting at 10 μM. Reactions were carried out at 10 μM ATP, 1 μM substrate cofilin, and 50 nM Limk1 (final concentrations). LIMK1 Kinase specific information: Genbank Accession # NP_002305; Recombinant catalytic domain (amino acids 285-638, His tagged, purified from insect cells), activated by co-expression of ROCK1. Reagents: Base Reaction buffer; 20 mM Hepes (pH 7.5), 10 mM MgCl₂, 1 mM EGTA, 0.02% Brij35, 0.02 mg/ml BSA, 0.1 mM Na₃VO₄, 2 mM DTT, 1% DMSO. No additional cofactors were added to the reaction mixture. Reaction Procedures: (a) Prepare indicated substrate in freshly prepared Base Reaction Buffer; (b) Deliver any required cofactors to the substrate solution above; (c) Deliver indicated kinase into the substrate solution and gently mix; (d) Deliver compounds in DMSO into the kinase reaction mixture; (e) Deliver ³³P-ATP (specific activity 0.01 μCi/μl final) into the reaction mixture to initiate the reaction; (f) Incubate kinase reaction for 120 min at room temperature; (g) Reactions are spotted onto P81 ion exchange paper (Whatman #3698-915); (h) Wash filters extensively in 0.1% Phosphoric acid.

Isolation of Resting CD4 T Cells from Peripheral Blood:

All protocols involving human subjects were reviewed and approved by the George Mason University institutional review board. Resting CD4 T cells were purified from peripheral blood of HIV-1 negative donors by two rounds of negative selection as previously described. Briefly, for the first-round depletion, we used monoclonal antibodies against human CD14, CD56 and HLA-DR, DP, and DQ (BD Biosciences). For the second-round depletion, we used monoclonal antibodies against human CD8, CD11b, and CD19 (BD Biosciences). Antibody-bound cells were depleted by using Dynabeads Pan Mouse IgG (Invitrogen). For further negative selection of the memory and naïve CD4 T cell subsets, monoclonal antibody against either CD45RA (0.02 μl per million cells) or CD45RO (0.1 μl per million cells) (BD Biosciences) was added during the second round of depletion. Purified cells were cultured in RPMI 1640 medium supplemented with 10% heat inactivated fetal bovine serum (Invitrogen), penicillin (50 U/ml) (Invitrogen), and streptomycin (50 μg/ml) (Invitrogen). Cells were rested overnight before infection or treatment.

### Viruses and Viral Infection:

Virus stocks of HIV-1(NL4-3) and HIV-1(AD8) were prepared by transfection of HeLa or HEK293T cells with cloned proviral DNA as described. HIV-1(VSVG) was prepared as described previously. Levels of p24 in the viral supernatant were measured in triplicate with ELISA using an in-house ELISA Kit. Viral titer (TCID50) was determined on the Rev-dependent GFP and Luciferase indicator cell Rev-CEM-GFP-Luc. For HIV infection of Rev-CEM-GFP-Luc, 2×10⁵ cells were treated with 100 μM of LIMK inhibitors for 1 hour, and then infected with 10³ to 10⁴⁵ TCID50 units of HIV-1 for 3 hours with the addition of LIMK inhibitors to maintain the drug concentration. Infected cells were washed twice, and then resuspended into 1 ml fresh medium without the addition of inhibitors. Cells were incubated for 2 days and viral infection was measured by flow cytometry (FACSCalibur, BD Biosciences) for GFP positive cells. To exclude drug cytotoxicity, propidum iodide (PI) (2 μg/ml, Fluka) was added into the cell suspension prior to flow cytometry, and only viable cells (PI negative) were used for measuring GFP expression. For luciferase assay, cells were resuspended in 100 μl of luciferase assay buffer (Promega), and measured using GloMax-Multi Detection System (Promega). For viral infection of resting CD4+ T cells, unless otherwise specified, 106 cells were treated by LIMK inhibitors for 1 hour. 10^{3.5} to 10^{4.5} TCID₅₀ units of HIV-1 were used to infect 10⁶ cells. During infection, LIMK inhibitors were added to maintain the drug concentration. Cells were washed once, and then resuspended into fresh medium (10⁶ cells per ml), and incubated for 5 days without stimulation. Cells were activated with anti-CD3/CD28 magnetic beads at 4 beads per cell. Culture supernatant (100 μl) was taken every two days or daily after stimulation. Cells were removed by centrifugation and supernatant saved for p24 ELISA. For HSV-1 infection, virus was propagated on Vero cells. Briefly, cells were infected with HSV-1 (MOI, 0.001) until 100% of cells displaying cytopathic effect (CPE). Cell supernatant with HSV-1 was harvested by centrifugation at 2,000 rpm for 5 min at 4° C., filtrated through 0.45 μm filter, and then stored at −80° C. For HSV-1 infection, Vero cells were seeded in 10 cm petri dishes and cultured overnight. SR10015 or DMSO was added to cells for 2 hours. HSV-1 was serially diluted with 199V medium, and added to cells for infection for 2 hours. Cells were washed and cultured in fresh medium (DMEM plus 5% NBCS, Invitrogen) containing 7.5 μg/ml pooled human immunoglobulin. Viral plaques were stained by rinsing twice in Phosphate-buffered saline with potassium. Cells were fixed with methanol, and stained with KaryoMax Giemsa Stain solution (Gibco). For infections with VEEV-Luc (TC83), VEEV(TC83) (BSL-2 strain), VEEV(TrD) (BSL-3 strain), or RVFV-Luc (MP12), Vero cells were pretreated with R10015 or DMSO, infected with the viruses at an MOI 0.1 and post-treated with R10015. Viral supernatants were collected 24 hours post-infection and analyzed by plaque assays. Alternatively for luciferase expressing viruses, luciferase activity was assessed at 24 hours post-expression and DMSO treated samples set to 100%. For EBOV(Zaire) infection, HFF-1 cells were pre-treated with R10015 for 2 hours, and infected with EBOV(Zaire) (MOI, 0.5). Infection was terminated at 48 hours post infection, and cells were fixed with formalin solution. Infected cells were identified by immunostaining of the EBOV GP protein with a primary mouse antibody and a secondary Alex488-labeled anti-mouse IgG antibody. The cells were also stained with Hoechst (Invitrogen) for nuclei and Cell Mask cytoplasm stain (Invitrogen) cytoplasm. The number of nuclei per well was used to determine cell viability. Images were taken by PE Opera confocal platform with 10× objective, and analyzed using Acapella and GeneData software.

### Western Blotting for LIMK, Cofilin and PAK1/2:

One million cells were lysed in NuPAGE LDS Sample Buffer (Invitrogen), separated by SDS-PAGE, and then transferred onto nitrocellulose membranes (Invitrogen). The membranes were washed in TBST for 3 min and then blocked for 30 min at room temperature with Starting Block blocking buffer (Pierce). For probing with different antibodies, blots were incubated with rabbit anti-LIMK1 antibodies (Cell Signaling), with rabbit anti-p-coflin antibodies (Cell Signaling) or with a mouse anti-PAK1/2 antibody (Cell Signaling). These antibodies were diluted in 2.5% milk/TBST and rocked overnight at 4° C. The blots were washed three times for 15 min, then incubated with either antirabbit or anti-mouse horseradish peroxidase-conjugated secondary antibodies (KPL) (1:5000) for 1 hour, and then developed with SuperSignal West Femto Maximum Sensitivity Substrate (Pierce). For loading control, the same blots were also stripped and reprobed with antibodies against GAPDH (Abcam) (1:1000). Images were captured with a CCD camera (FluorChem 9900 Imaging Systems) (Alpha Innotech).

### Surface Staining of CD4 and CXCR4:

Cells were stained with FITC-labeled monoclonal antibody against human CD4 (clone PRA-T4) or CXCR4 (clone 12G5) (BD Biosciences). Cells were stained on ice in PBS+0.1% BSA for 30 minutes, washed with cold PBS-0.5% BSA, and then analyzed on a FACSCalibur (BD Biosciences).

### Quantitative Real-Time PCR:

HIV viral DNA was quantified using the Bio-Rad iQ5 real-time PCR detection system, utilizing the forward primer 5′LTR-U5, the reverse primer 3′ gag, and the probe FAM-U5/gag. Pre-qualified, full-length proviral plasmid pNL4-3 was used as the DNA standard. Viral DNA and 2-LTR circles were measured as described previously 2. For measuring 2-LTR-circles, the DNA was amplified by real-time PCR with primers and probe MH536, MH535, and MH603.

### Chemotaxis Assay:

A half million Jurkat T cells were resuspended into 100 μl RPMI-1640 medium and then added to the upper chamber of a 24-well transwell plate (Corning). The lower chamber was filled with 600 μl of medium premixed with SDF-1 (40 ng/ml). The plate was incubated at 37° C. for 2 hours, and then the upper chamber was removed and cells in the lower chamber were counted. Where indicated, different concentrations of R10015 were added to the culture supernatant prior to the assay along with a DMSO control.

### FITC-Phalloidin Staining of F-Actin and Flow Cytometry:

F-actin staining using phalloidin-FITC (Sigma) was carried out as described previously. Briefly, each staining was carried out using 1×10⁶ cells. Cells were pelleted, fixed, and permeabilized with CytoPerm/Cytofix buffer (BD Biosciences) for 20 min at room temperature and washed with cold Perm/Wash buffer (BD Biosciences) twice, followed by staining with 5 μl of 0.3 mM FITC-labeled phalloidin (Sigma) for 30 min on ice in the dark. Cells were washed twice with cold Perm/Wash buffer, then were resuspended in 1% paraformaldehyde, and analyzed on a FACSCalibur (BD Biosciences).

### Viral Entry Assays:

The BlaM-Vpr-based viral entry assay was performed as previously described. We also used a Nef-luciferase-based entry assay as described. Briefly, cells (1×10⁶) were treated by 100 μM R10015 for 1 hour, and then infected with 200 ng of Nef-luciferase containing viruses at 37° C. for 3 hours with the same R10015 concentration, and then washed three times with medium. Cells were resuspended in 100 μl of luciferase assay buffer (Promega), and luciferase activity was measured in live cells using a GloMax-Multi Detection System (Promega).

### Surface Staining of CD25 and CD69:

A half million resting CD4 T cells were cultured for 5 days and stimulated with anti-CD3/CD28 beads (4 beads per cell) for 24 hours. Cells were stained with PE-labeled monoclonal antibody against human CD25 (clone RpA-T4) or CD69 (clone 12G5) (BD Biosciences). Cells were stained on ice in PBS+0.1% BSA for 30 minutes, washed with cold PBS-0.5% BSA, and then analyzed on a FACSCalibur (BD Biosciences).

Docking of Limk Inhibitors into a Crystal Structure of Limk1:

Inhibitor 18b was prepared for glide docking using LigPrep (Schrodinger, LLC, NY). The chain A of PDB ID 3S95 was prepared using protein preparation wizard in Maestro V 9.8 (Schrodinger, LLC, NY) by removing water molecules and bound ligand, and adding hydrogen atoms. The docking grid was generated around the original ligand with a box size of 18×18×18 Å³. Docking was conducted without any constraint. The top scored docking pose was merged to the protein for energy minimization using Prime (Schrodinger, LLC, NY).

### Limk1 Biochemical Assays and Kinase Profiling:

Biochemical assays for all Limk inhibitors and kinase profiling were carried out in Reaction Biology Corporation and followed the protocols described on its website. Compounds were tested in 10-dose IC₅₀ mode with 3-fold series dilution starting at 10 μM for IC₅₀ measurements. Compounds were tested at 1 μM with duplicate experiments in profiling assays. Control compound Staurosporine was tested in 10-dose IC₅₀ mode with 3-fold serial dilution starting at 10 μM. Reactions were carried out at 10 μM ATP, 1 μM substrate cofilin, and 50 nM Limk1 (final concentrations).

### In-Cell Western Assay in A7r5 Cells:

A7r5 (15,000 cells/well) were plated in a clear-bottomed Packard View black 96-well plate in 100 μl of 10% FBS DMEM:F12 medium and were allowed to attach overnight. The next day, the cells were serum starved in 1% FBS DMEM medium for 2 hr and then treated with the compounds for 1 hr. Cells were then fixed in 4% paraformaldehyde in PBS for 20 min at room temperature (RT) with no shaking They were then washed once with 0.1 M glycine to neutralize paraformaldehyde for 5 min Cells were permeabilized with 0.2% Triton X-100 in PBS for 20 min at RT on orbital shaker after which they were washed once with PBS for 5 min They were then incubated with Licor Blocking Buffer in PBS (1:1 dilution in PBS) for 1-1.5 h rocking at RT. Cells were incubated with primary antibody p-cofilin Ab (Cell Signaling #3311) 1:100 dilution in Licor blocking buffer overnight at 4° C. Next day, they were washed twice with PBS-0.1% Tween 20 (PBST) washing solution for 5 min each at room temperature on the orbital shaker, followed by one wash with Licor Blocking Buffer containing 0.05% Tween-20 for 5 min on the shaker at RT. The cells were then incubated with secondary antibody goat anti-rabbit IR800 (1:500 dilution) for 1 hr at RT in the dark (covered the plate with foil) in Licor blocking buffer-containing Tween-20. Following this, cells were washed twice with PBST for 5 min each at room temperature and then once with Licor blocking buffer-containing 0.05% Tween-20. The wells were then incubated with ToPro 3 stain (nucleic acid staining), diluted 1:4000 in Licor blocking buffer or Licor blocking buffer with 0.05% Tween-20 for 30 min at room temperature in the dark. Finally the plates were washed twice with PBS and analyzed using the Odyssey LICOR Infrared Scanner.

### Cofilin Phosphorylation Cell Assay in PC-3 Cell Lines:

PC3 cells were cultured at a density of 0.5×10⁶ cells/mL in 60 mm culture dishes in 10% FBS RPMI1640 media. Then, the cells were treated with DMSO and the indicated concentration of Limk inhibitors. After incubating for 24 h, the cells were rinsed with ice-cold PBS twice and collected by spinning down at 4° C. in 10,000 rpm for 5 min Cellular lysates were prepared by suspending cells in SDS sample buffer, 120 mmol/L Tris, 4% SDS, 20% glycerol, 0.1 mg/mL bromophenol blue, and 100 mmol/L DTT (pH 6.8). After brief sonication, the lysates were heated at 95° C. for 5 minutes. The cell lysates were separated by 12% SDS-PAGE and transferred to Immobilon-P membranes (Millipore Corp) Immunostaining was done using antibodies specific for phospho-Cofilin (Cell Signaling, #3313) and β-Actin (GeneScript, #A00702) antibodies and the corresponding second antibodies for whole immunoglobulins from mouse or rabbit (Amersham Biosciences). Immunoreactive proteins were detected by chemoluminescence using the Pierce ECL Western Blotting Substrate (Thermo Scientific). We quantified the actual levels of proteins by using the Multigauge ver 3.0 software (Fujifilm). The gels were stained with Coomassie Brilliant Blue R-250 (0.25%) for 1 hour and then destained (all solutions from Bio-Rad) to check the loading amount of protein samples on the gels.

### Cofilin Phosphorylation Cell Assay in CEM-SS T Cell Lines:

CEM-SS T cells (1.0×10⁶) were treated with a Limk inhibitor at 10 μM and 1 μM separately at 37° C. for 4 h. Cells were lysed in NuPAGE LDS Sample Buffer (Invitrogen) followed by sonication. Samples were heated at 90° C. for 10 minutes, separated by SDS-PAGE, and then transferred onto nitrocellulose membranes (Invitrogen). The membranes were washed in TBST for 3 minutes and then blocked for 30 minutes at room temperature with Starting Block blocking buffer (Pierce). The blots were incubated with a rabbit anti-phospho cofilin (ser3) antibody (1:500 dilution) (Cell Signaling) diluted in 2.5% milk-TBST and rocked overnight at 4° C. The blots were washed three times for 15 minutes, then incubated with goat anti-rabbit 800cw labeled antibodies (Li-cor Biosciences) (1:5000 diluted in blocking buffer) for 1 h at 4° C. The blots were washed three times for 15 minutes and scanned with Odyssey Infrared Imager (Li-cor Biosciences). The same blots were also stripped and reprobed with antibodies against GAPDH (Abcam) as a loading control.

### In Vitro Invasion Assay in PC-3 Cells:

Transwell chambers coated with GFR Matrigel (BD Biosciences) were used for measurement of cell invasion. The matrigel was solidified at 37° C. in a humidified incubator the day before the assay. PC3 cells (1×10³ cells per well) were grown in serum-free RPMI1640 media in the upper side of the insert. The lower well was filled with RPMI 1640 supplemented with 10% FBS with a Limk inhibitor (1 μM). PC3 cells were incubated at 37° C. in a humidified atmosphere containing 5% CO₂ for 48 h. Then transwell membrane was rinsed three times with PBS, and the cells were fixed in 2.5% EM grade glutaraldehyde for 15 min at room temperature (RT) with no shaking. After removing glutaraldehyde by aspirating, the cells were permeabilized with 0.5% Triton X-100 in PBS for 3 min at RT, and they were rinsed three times with PBS. Then, the cells were stained by Gill's hematoxylin No. 1 for 15 min at RT, and they were washed by distilled water for three times. To remove any residual stain, the cells were washed by acid alcohol for 3 minutes and then rinsed by distilled water twice. The cells were exposed with 0.04% NH₄OH until a blue color is observed on the membrane and then rinsed by distilled water twice. The membranes were dried overnight, and the stained cells were visualized under Leica DMI3000B microscope.

### In Vitro Migration Assay in PC-3 Cells:

PC3 cells were cultured to confluence at >90% in 6 well culture dishes in 10% FBS RPMI1640 media the day before the assay. Lines were drawn with a marker on the bottom of the dishes. Using a sterile 1 mL pipet tip, the dishes were scratched three separate wounds through the cells moving perpendicular to the line drawn in the step above. The cells were rinsed with PBS twice very gently and added the RPMI1640 media, and the dishes were taken pictures using phase contrast under Leica DMI3000B microscope. Then, the cells were treated with the indicated concentration of a Limk Inhibitor and incubated for 24 h. The dishes were taken pictures to detect closed wound area as describe above, and the closed wound area was analyzed by ImageJ software (Ver 1.48).

### Evaluations

It is within ordinary skill to evaluate any compound disclosed and claimed herein for effectiveness in inhibition of LIM kinase and in the various cellular assays using the procedures described above or found in the scientific literature. Accordingly, the person of ordinary skill can prepare and evaluate any of the claimed compounds without undue experimentation.

Any compound found to be an effective inhibitor of LIM kinase can likewise be tested in animal models and in human clinical studies using the skill and experience of the investigator to guide the selection of dosages and treatment regimens.

### CITED DOCUMENTS

1. Bernard, O.; Ganiatsas, S.; Kannourakis, G.; Dringen, R. Kiz-1, a protein with LIM zinc finger and kinase domains, is expressed mainly in neurons. Cell Growth Differ. 1994, 5, 1159-1171.
2. Stanyon, C. A.; Bernard, O. LIM-kinasel. Int. J. Biochem. Cell Biol. 1999, 31, 389-394.
3. Mizuno, K.; Okana, I.; Ohashi, K.; Nunoue, K.; Kuma, K.; Miyata, T.; Nakamura, T. Identification of a human cDNA encoding a novel protein kinase with two repeats of the LIM/double Zinc finger motif. Oncogene 1994, 9, 1605-1612.
4. Osada, H.; Hasada, K.; Inazawa, J.; Uchida, K.; Ueda, R.; Takahashi, T.; Takahashi, T. Subcellular localization and protein interaction of the human LIMK2 gene expressing alternative transcripts with tissue-specific regulation. Biochem. Biophys. Res. Commun. 1996, 229, 582-589.
5. Bernard, O. Lim kinases, regulators of actin dynamics. Int. J. Biochem. Cell Biol. 2007, 39, 1071-1076.
6. Acevedo, K.; Moussi, N.; Li, R.; Soo, P.; Bernard, O. LIM kinase 2 is widely expressed in all tissues. J. Histochem. Cytochem. 2006, 54, 487-501.
7. Scott, R. W.; Olson, M. F. LIM kinases: function, regulation and association with human disease. J. Mol. Med. (Berl) 2007, 85, 555-568.
8. Arber, S.; Barbayannis, F. A.; Hanser, H.; Schneider, C.; Stanyon, C. A.; Bernard, O.; Caroni, P. Regulation of actin dynamics through phosphorylation of cofilin by LIM-kinase. Nature 1998, 393, 805-809.
9. Edwards, D. C.; Sanders, L. C.; Bokoch, G. M.; Gill, B. N. Activation of LIM-kinased by Pak1 couples Rac/Cdc42 GTPase signaling to actin cytoskeletal dynamics. Nat. Cell Biol. 1999, 1, 253-259.
10. Maekawa, M.; Ishizaki, T.; Boku, S.; Watanabe, N.; Fujita, A.; Iwamatsu, A.; Obinata, T.; Ohashi, K.; Mizuno, K.; Narumiya, S. Signaling from Rho to the actin cytoskeleton through protein kinases ROCK and LIM-kinase. Science 1999, 285, 895-898.
11. Vlachos, P.; Joseph, B. The Cdk inhibitor p57(Kip2) controls LIM-kinase 1 activity and regulates actin cytoskeleton dynamics. Oncogene 2009, 28, 4175-4188.
12. Thirone, A. C.; Speight, P.; Zulys, M.; Rotstein, O. D.; Szaszi, K.; Pedersen, S. F.; Kapus, A. Hyperosmotic stress induces Rho/Rho kinase/LIM kinase-mediated cofilin phosphorylation in tubular cells: key role in the osmotically triggered F-actin response. Am. J. Physiol. Cell Physiol. 2009, 296, C463-475.
13. Hoogenraad, C. C.; Akhmanova, A.; Galjart, N.; De Zeeuw, C. I. LIMK1 and CLIP-115: linking cytoskeletal defects to Williams syndrome. Bioessays 2004, 26, 141-150.
14. Piccioli, Z. D.; Littleton, J. T. Retrograde BMP signaling modulates rapid activity-dependent synaptic growth via presynaptic LIM kinase regulation of cofilin. J. Neurosci. 2014, 34, 4371-4381.
15. Heredia, L.; Helguera, P.; de Olmos, S.; kedikian, G.; Sola Vigo, F.; LaFerla, F. e. a. Phosphorylation of actin-depolymerizing factor/cofilin by Lim-kinase mediates amylolid beta-induced degeneration: a potential mechanism of neuronal dystrophy in Alzheimer's disease. J. Neurosci. 2006, 26, 6533-6542.
16. Honma, M.; Benitah, S. A.; Watt, F. M. Role of LIM kinases in normal and psoriatic human epidermis. Mol. Biol. Cell 2006, 17, 1888-1896.
17. Bongalon, S.; Dai, Y. P.; Singer, C. A.; Yamboliev, I. A. PDGF and IL-1beta upregulate cofilin and LIMK2 in canine cultured pulmonary artery smooth muscle cells. J. Vasc. Res. 2004, 41, 412-421.
18. Dai, Y. P.; Bongalon, S.; Tian, H.; Parks, S. D.; Mutafova-Yambolieva, V. N.; Yamboliev, I. A. Upregulation of profilin, cofilin-2 and LIMK2 in cultured pulmonary artery smooth muscle cells and in pulmonary arteries of monocrotaline-treated rats. Vascul. Pharmacol. 2006, 44, 275-282.
19. Akagawa, H.; Tajima, A.; Sakamoto, Y.; Krischek, B.; Yoneyama, T.; Kasuya, H.; Onda, H.; Hori, T.; Kubota, M.; Machida, T.; Saeki, N.; Hata, A.; Hashiguchi, K.; Kimura, E.; Kim, C. J.; Yang, T. K.; Lee, J. Y.; Kimm, K.; Inoue, I. A haplotype spanning two genes, ELN and LIMK1, decreases their transcripts and confers susceptibility to intracranial aneurysms. Hum. Mol. Genet. 2006, 15, 1722-1734.
20. Harrison, B. A.; Whitlock, N. A.; Voronkov, M. V.; Almstead, Z. Y.; Gu, K. J.; Mabon, R.; Gardyan, M.; Hamman, B. D.; Allen, J.; Gopinathan, S.; McKnight, B.; Crist, M.; Zhang, Y.; Liu, Y.; Courtney, L. F.; Key, B.; Zhou, J.; Patel, N.; Yates, P. W.; Liu, Q.; Wilson, A. G.; Kimball, S. D.; Crosson, C. E.; Rice, D. S.; Rawlins, D. B. Novel class of LIM-kinase 2 inhibitors for the treatment of ocular hypertension and associated glaucoma. J. Med. Chem. 2009, 52, 6515-6518.
21. Xu, X.; Guo, J.; Vorster, P.; Wu, Y. Involvement of LIM kinase 1 in actin polarization in human CD4 T cells. Commun. Integr. Biol. 2012, 5, 381-383.
22. Manetti, F. HIV-1 proteins join the family of LIM kinase partners. New roads open up for HIV-1 treatment. Drug Discov. Today 2012, 17, 81-88.
23. Vorster, P. J.; Guo, J.; Yoder, A.; Wang, W.; Zheng, Y.; Xu, X.; Yu, D.; Spear, M.; Wu, Y. LIM kinase 1 modulates cortical actin and CXCR4 cycling and is activated by HIV-1 to initiate viral infection. J. Biol. Chem. 2011, 286, 12554-12564.
24. Wen, X.; Ding, L.; Wang, J. J.; Qi, M.; Hammonds, J.; Chu, H.; Chen, X.; Hunter, E.; Spearman, P. ROCK1 and LIM kinase modulate retrovirus particle release and cell-cell transmission events. J. Virol. 2014, 88, 6906-6921.
25. Bagheri-Yarmand, R.; Mazumdar, A.; Sahin, A. A.; Kumar, R. LIM kinase 1 increases tumor metastasis of human breast cancer cells via regulation of the urokinase-type plasminogen activator system. Int. J. Cancer 2006, 118, 2703-2710.
26. Davila, M.; Frost, A. R.; Grizzle, W. E.; Chakrabarti, R. LIM kinase 1 is essential for the invasive growth of prostate epithelial cells: implications in prostate cancer. J. Biol. Chem. 2003, 278, 36868-36875.
27. Davila, M.; Jhala, D.; Ghosh, D.; Grizzle, W. E.; Chakrabarti, R. Expression of LIM kinase 1 is associated with reversible G1/S phase arrest, chromosomal instability and prostate cancer. Mol. Cancer 2007, 6, 40.
28. Manetti, F. LIM kinases are attractive targets with many macromolecular partners and only a few small molecule regulators. Med. Res. Rev. 2012, 32, 968-998.
29. Suyama, E.; Wadhwa, R.; Kawasaki, H.; Yaguchi, T.; Kaul, S. C.; Nakajima, M.; Taira, K. LIM kinase-2 targeting as a possible anti-metastasis therapy. J. Gene Med. 2004, 6, 357-363.
30. Manetti, F. Recent findings confirm LIM domain kinases as emerging target candidates for cancer therapy. Curr. Cancer Drug Targets 2012, 12, 543-560.
31. Ohashi, K.; Sampei, K.; Nakagawa, M.; Uchiumi, N.; Amanuma, T.; Aiba, S.; Oikawa, M.; Mizuno, K. Damnacanthal, an effective inhibitor of LIM-kinase, inhibits cell migration and invasion. Mol. Biol. Cell 2014, 25, 828-840.
32. Park, J. B.; Agnihotri, S.; Golbourn, B.; Bertrand, K. C.; Luck, A.; Sabha, N.; Smith, C. A.; Byron, S.; Zadeh, G.; Croul, S.; Berens, M.; Rutka, J. T. Transcriptional profiling of GBM invasion genes identifies effective inhibitors of the LIM kinase-Cofilin pathway. Oncotarget 2014.
33. Rak, R.; Kloog, Y. Targeting LIM kinase in cancer and neurofibromatosis. Cell Cycle 2014, 13, 1360-1361.
34. Mashiach-Farkash, E.; Rak, R.; Elad-Sfadia, G.; Haklai, R.; Carmeli, S.; Kloog, Y.; Wolfson, H. J. Computer-based identification of a novel LIMK1/2 inhibitor that synergizes with salirasib to destabilize the actin cytoskeleton. Oncotarget 2012, 3, 629-639.
35. Iyengar, S.; Hildreth, J. E.; Schwartz, D. H. Actin-dependent receptor colocalization required for human immunodeficiency virus entry into host cells. J. Virol. 1998, 72, 5251-5255.
36. Ross-Macdonald, P.; de Silva, H.; Guo, Q.; Xiao, H.; Hung, C. Y.; Penhallow, B.; Markwalder, J.; He, L.; Attar, R. M.; Lin, T. A.; Seitz, S.; Tilford, C.; Wardwell-Swanson, J.; Jackson, D. Identification of a nonkinase target mediating cytotoxicity of novel kinase inhibitors. Mol. Cancer Ther. 2008, 7, 3490-3498.
37. He, L.; Seitz, S. P.; Trainor, G. L.; Tortolani, D.; Vaccaro, W.; Poss, M.; Tarby, C. M.; Tokarski, J. S.; Penhallow, B.; Hung, C. Y.; Attar, R.; Lin, T. A. Modulation of cofilin phosphorylation by inhibition of the Lim family kinases. Bioorg. Med. Chem. Lett. 2012, 22, 5995-5998.
38. Sleebs, B. E.; Nikolakopoulos, G.; Street, I. P.; Falk, H.; Baell, J. B. Identification of 5,6-substituted 4-aminothieno[2,3-d]pyrimidines as LIMK1 inhibitors. Bioorg. Med. Chem. Lett. 2011, 21, 5992-5994.
39. Sleebs, B. E.; Ganame, D.; Levit, A.; Street, I. P.; Gregg, A.; Falkabc, H.; Baell, J. B. Development of substituted 7-phenyl-4-aminobenzothieno[3,2-d] pyrimidines as potent LIMK1 inhibitors. Med. Chem. Commun. 2011, 2, 982-986.
40. Goodwin, N. C.; Cianchetta, G.; Burgoon, H. A.; Healy, J.; Mabon, R.; Strobel, E. D.; Allen, J.; Wang, S.; Hamman, B. D.; Rawlins, D. B. Discovery of a Type III Inhibitor of LIM Kinase 2 That Binds in a DFG-Out Conformation. ACS Med Chem Lett 2014, dx.doi.org/10.1021/ml500242y.
41. Yin, Y.; Cameron, M. D.; Lin, L.; Khan, S.; Schröter, T.; Grant, W.; Pocas, J.; Chen, Y. T.; Schürer, S.; Pachori, A.; LoGrasso, P.; Feng, Y. Discovery of Highly Potent and Selective Urea-based ROCKInhibitors and Their Effects on Intraocular Pressure in Rats. ACS Med. Chem. Lett. 2010, 1, 175-179.
42. Yin, Y.; Lin, L.; Ruiz, C.; Khan, S.; Cameron, M. D.; Grant, W.; Pocas, J.; Eid, N.; Park, H.; Schroter, T.; Lograsso, P. V.; Feng, Y. Synthesis and biological evaluation of urea derivatives as highly potent and selective rho kinase inhibitors. J. Med. Chem. 2013, 56, 3568-3581.
43. Morita, Y.; Ishigaki, T.; Kawamura, K.; Iseki, K. Short and Practical Synthesis of N′,N′-disubstituted N-Aryl-1,2-ethylenediamines by a Decarboxylative Ring-opening Reaction Under Nucleophilic Conditions. Synthesis 2007, 16, 2517-2523.
44. Goodacre, C. J.; Bromidge, S. M.; Clapham, D.; King, F. D.; Lovell, P. J.; Allen, M.; Campbell, L. P.; Holland, V.; Riley, G. J.; Starr, K. R.; Trail, B. K.; Wood, M. D. A series of bisaryl imidazolidin-2-ones has shown to be selective and orally active 5-HT2C receptor antagonists. Bioorg. Med. Chem. Lett. 2005, 15, 4989-4993.
45. Beletskaya, I. P.; Bessmertnykh, A. G.; Guilard, R. Halo-substituted Aminobenzenes Prepared by Pd-catalyzed Amination. Synlett. 1999, 9, 1459-1461.
46. Feng, Y.; Yin, Y.; Weiser, A.; Griffin, E.; Cameron, M. D.; Lin, L.; Ruiz, C.; Schürer, S. C.; Inoue, T.; Rao, P. V.; Schröter, T.; LoGrasso, P. Discovery of Substituted 4-(Pyrazol-4-yl)-phenylbenzodioxane-2-carboxamides as Potent and Highly Selective Rho Kinase (ROCK-II) Inhibitors. J. Med. Chem. 2008, 51, 6642-6645.
47. Schröter, T.; Griffin, E.; Weiser, A.; Feng, Y.; LoGrasso, P. Detection of myosin light chain phosphorylation—A cell-based assay for screening Rho-kinase inhibitors. Biochem. Biophys. Res. Commun. 2008, 374, 356-360.
48. Feng, Y.; Cameron, M. D.; Frackowiak, B.; Griffin, E.; Lin, L.; Ruiz, C.; Schroter, T.; LoGrasso, P. Structure-activity relationships, and drug metabolism and pharmacokinetic properties for indazole piperazine and indazole piperidine inhibitors of ROCK-II. Bioorg. Med. Chem. Lett. 2007, 17, 2355-2360.
49. Feng, Y.; Chambers, J. W.; Iqbal, S.; Koenig, M.; Park, H.; Cherry, L.; Hernandez, P.; Figuera-Losada, M.; LoGrasso, P. V. A small molecule bidentate-binding dual inhibitor probe of the LRRK2 and JNK kinases. ACS Chem. Biol. 2013, 8, 1747-1754.
50. Chen, Y. T.; Bannister, T. D.; Weiser, A.; Griffin, E.; Lin, L.; Ruiz, C.; Cameron, M. D.; Schürer, S.; Duckett, D.; Schröter, T.; LoGrasso, P.; Feng, Y. Chroman-3-amides as potent Rho kinase inhibitors. Bioorg. Med. Chem. Lett. 2008, 18, 6406-6409.
51. Ahmed, T.; Shea, K.; Masters, J. R.; Jones, G. E.; Wells, C. M. A PAK4-LIMK1 pathway drives prostate cancer cell migration downstream of HGF. Cell Signal. 2008, 20, 1320-1328.
52. Yin, Y.; Lin, L.; Ruiz, C.; Cameron, M. D.; Pocas, J.; Wayne, G.; Schröter, T.; Chen, W.; Duckett, D.; Schürer, S. C.; LoGrasso, P.; Feng, Y. Benzothiazole as Rho-associated Kinase (ROCK-II) Inhibitors. Bioorg. Med. Chem. Lett. 2009, 19, 6686-6690.
53. Fang, X.; Yin, Y.; Wang, B.; Yao, L.; Chen, Y. T.; Schröter, T.; Weiser, A.; Pocas, J.; Wayne, G.; Cameron, M. D.; Lin, L.; Ruiz, C.; Khan, S.; Schürer, S. C.; Pachori, A.; LoGrasso, P.; Feng, Y. Tetrahydroisoquinoline derivatives as potent and selective Rho kinase Inhibitors. J. Med. Chem. 2010, 53, 5727-5737.
54. Chowdhury, S.; Chen, Y. T.; Fang, X.; Grant, W.; Pocas, J.; Cameron, M. D.; Ruiz, C.; Lin, L.; Park, H.; Schröter, T.; Bannister, T. D.; LoGrasso, P. V.; Feng, Y Amino Acid Derived Quinazolines as Rock/PKA Inhibitors. Bioorg. Med. Chem. Lett. 2013, 23, 1592-1599.
55. Zheng, K.; Iqbal, S.; Hernandez, P.; Park, H.; LoGrasso, P.; Feng, Y. Design and Synthesis of Highly Potent and Isoform Selective JNK3 Inhibitors: SAR Studies on Aminopyrazole Derivatives. J. Med. Chem. 2014, dx.doi.org/10.1021/jm501256y.
56. Zhou, P.; Zou, J. W.; Tian, F. F.; Shang, Z. C. Fluorine Bonding; How Does It Work In Protein-Ligand Interactions? J. Chem. Inf. Model. 2009, 49, 2344-2355.
57. Prudent, R.; Vassal-Stermann, E.; Nguyen, C. H.; Pillet, C.; Martinez, A.; Prunier, C.; Barette, C.; Soleilhac, E.; Filhol, O.; Beghin, A.; Valdameri, G.; Honore, S.; Aci-Seche, S.; Grierson, D.; Antonipillai, J.; Li, R.; Di Pietro, A.; Dumontet, C.; Braguer, D.; Florent, J. C.; Knapp, S.; Bernard, O.; Lafanechere, L. Pharmacological inhibition of LIM kinase stabilizes microtubules and inhibits neoplastic growth. Cancer Res. 2012, 72, 4429-4439.
58. Yoshioka, K.; Foletta, V.; Bernard, O.; Itoh, K. A role for LIM kinase in cancer invasion. Proc. Natl. Acad. Sci. USA 2003, 100, 7247-7252.
59. Wu, Y.; Beddall, M. H.; Marsh, J. W. Rev-dependent lentiviral expression vector. Retrovirology 2007, 4, 12.
60. Wu, Y.; Beddall, M. H.; Marsh, J. W. Rev-dependent indicator T cell line. Curr. HIV Res. 2007, 5, 394-402.
61. Mishima, T.; Naotsuka, M.; Horita, Y.; Sato, M.; Ohashi, K.; Mizuno, K. LIM-kinase is critical for the mesenchymal-to-amoeboid cell morphological transition in 3D matrices. Biochem. Biophys. Res. Commun. 2010, 392, 577-581.
62. Horita, Y.; Ohashi, K.; Mukai, M.; Inoue, M.; Mizuno, K. Suppression of the invasive capacity of rat ascites hepatoma cells by knockdown of Slingshot or LIM kinase. J. Biol. Chem. 2008, 283, 6013-6021.
63. Alers, J. C.; Rochat, J.; Krijtenburg, P. J.; Hop, W. C.; Kranse, R.; Rosenberg, C.; Tanke, H. J.; Schroder, F. H.; van Dekken, H. Identification of genetic markers for prostatic cancer progression. Lab Invest. 2000, 80, 931-942.
64. Yang, N.; Higuchi, O.; Ohashi, K.; Nagata, K.; Wada, A.; Kangawa, K.; Nishida, E.; Mizuno, K. Cofilin phosphorylation by LIM-kinase 1 and its role in Rac-mediated actin reorganization. Nature 1998, 393, 809-812.
65. Ying, H.; Biroc, S. L.; Li, W. W.; Alicke, B.; Xuan, J. A.; Pagila, R.; Ohashi, Y.; Okada, T.; Kamata, Y.; Dinter, H. The Rho kinase inhibitor fasudil inhibits tumor progression in human and rat tumor models. Mol. Cancer Ther. 2006, 5, 2158-2164.
66. Hopkins, A. M.; Pineda, A. A.; Winfree, L. M.; Brown, G. T.; Laukoetter, M. G.; Nusrat, A. Organized migration of epithelial cells requires control of adhesion and protrusion through Rho kinase effectors. Am. J. Physiol. Gastrointest. Liver Physiol. 2007, 292, G806-817.
67. Nine week old Brown Norway rats (n=6/group) were housed under constant low light conditions for 65 days (illuminance ranging from 60-80 lux). At 17 weeks, 20 ul of 18w (0.25%) was applied to the right eye. The left eye was untreated and served as the control. IOP measurements were made on both eyes prior to and at 1, 4, 7 and 24 hours post administration. A Tonolab pen was used for IOP measurements. Initial baseline IOP of the treated eye was 28 mmHg. The maximal IOP decrease of the treated eye was ˜6 mmHg from 1 to 4 hours.

All patents and publications referred to herein are incorporated by reference herein to the same extent as if each individual publication was specifically and individually indicated to be incorporated by reference in its entirety.

The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

It is well known that for international applications, color drawings and photographs are not accepted. The public is directed to the US Patent Application Information Retrieval (PAIR) website, which upon querying U.S. Provisional Application No. 62/338,040, filed May 18, 2016, the public may access color versions of the figures of this application.

1. A method of modulating a LIM kinase, comprising contacting the LIM kinase with an effective amount or concentration of a compound of formula (I) 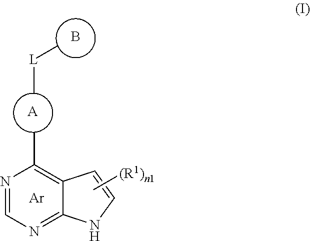
wherein
R¹ is halo, cyano (C₁-C₆)alkoxy, (C₁-C₆)alkyl, (C₂-C₆)alkenyl, or (C₂-C₆)alkynyl, wherein any alkyl, alkenyl, or alkynyl can be unsubstituted or substituted; wherein any one or two methylene groups thereof can be substituted with any of an independently selected NR′, S, O, C(═S), C(═O), OC(═O), OC(═O)O, OC(═O)NR′, C(═O)C(═O), SO₂NR′, S(O), S(O)₂, or C(═O)NR′NR′, wherein each independently selected R′ is H or (C₁-C₆)alkyl or (C₃-C₇)cycloalkyl;
n1=0, 1, or 2;
ring A is a 6-membered saturated ring comprising one or two nitrogen atoms, wherein the nitrogen atoms are disposed at the positions of ring A bonded to linker L, or to ring B when L is a direct bond, and to the ring system Ar; or ring A is a 5- or 6-membered aryl ring, a 5- or 6-membered heteroaryl ring, or a fused 6:5 heteroaryl ring system; ring A is substituted with n2 R² groups, wherein R² is halo, halo(C₁-C₆)alkyl, cyano, (C₁-C₆)alkoxy, (C₁-C₆)alkyl, (C₂-C₆)alkenyl, or (C₂-C₆)alkynyl, wherein any alkyl, alkenyl, or alkynyl can be unsubstituted or substituted; wherein any one or two methylene groups thereof can be substituted with any of an independently selected NR′, S, O, C(═S), C(═O), OC(═O), OC(═O)O, OC(═O)NR′, C(═O)C(═O), SO₂NR′, S(O), S(O)₂, or C(═O)NR′NR′, wherein each independently selected R′ is H or (C₁-C₆)alkyl or (C₃-C₇)cycloalkyl;
n2=0, 1, or 2;
L is a direct bond between ring A and ring B, or L is N(R³)C(═O)N(R³), wherein each R³ is independently H or (C₁-C₆)alkyl, wherein the R³ alkyl can be substituted with hydroxyl, (C₁-C₆)alkoxyl, amino, mono- or di-(C₁-C₆)alkylamino, or a 4- to 7-membered heterocyclyl ring;
ring B is a 5- or 6-membered aryl ring, a 5-membered or a 6-membered heteroaryl ring, or a fused 6:5 heteroaryl ring system; ring B is substituted with n4 R⁴ groups, wherein R⁴ is halo, cyano, (C₁-C₆)alkyl, (C₂-C₆)alkenyl, (C₂-C₆)alkynyl, (C₁-C₆)alkoxy, (C₁-C₆)alkoxycarbonyl, (C₃-C₇)cycloalkoxycarbonyl, R′NHC(═O), or R′₂NC(═O), wherein any alkyl, alkenyl, or alkynyl can be unsubstituted or substituted; wherein any one or two methylene groups thereof can be substituted with any of an independently selected NR′, S, O, C(═S), C(═O), OC(═O), OC(═O)O, OC(═O)NR′, C(═O)C(═O), SO₂NR′, S(O), S(O)₂, or C(═O)NR′NR′, wherein each independently selected R′ is H or (C₁-C₆)alkyl or (C₃-C₇)cycloalkyl; or a pharmaceutically acceptable salt thereof.

2. The method of claim 1, wherein the compound of formula (I) is a compound of formula (IA) 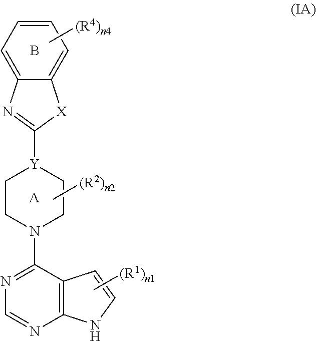
wherein
Y is CR³ or N;
X is NR³, CH₂, CHR¹, O, or S;
wherein R¹, n1, R², n2, R³, R⁴, and n4, are as defined in claim 1.

3. The method of claim 1, wherein the compound of formula (I) is a compound of formula (IB) 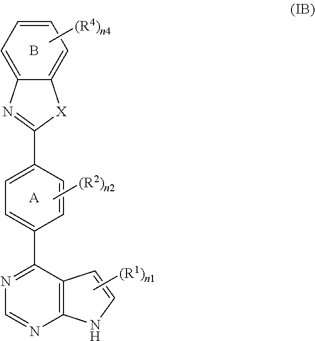
X is NR³, CH₂, CHR¹, O, or S;
wherein R¹, n1, R², n2, R³, R⁴, and n4, are as defined in claim 1.

4. The method of claim 1, wherein the compound of formula (I) is 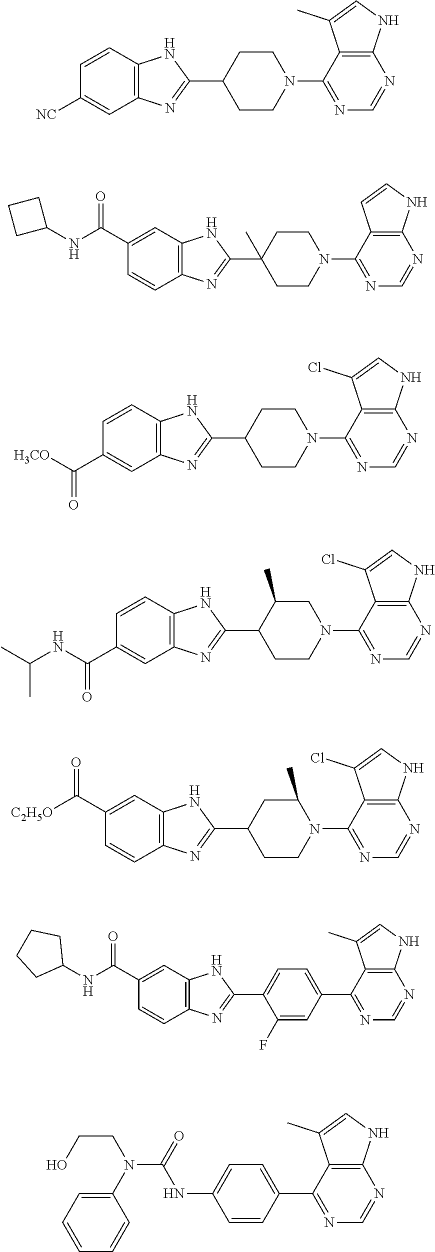 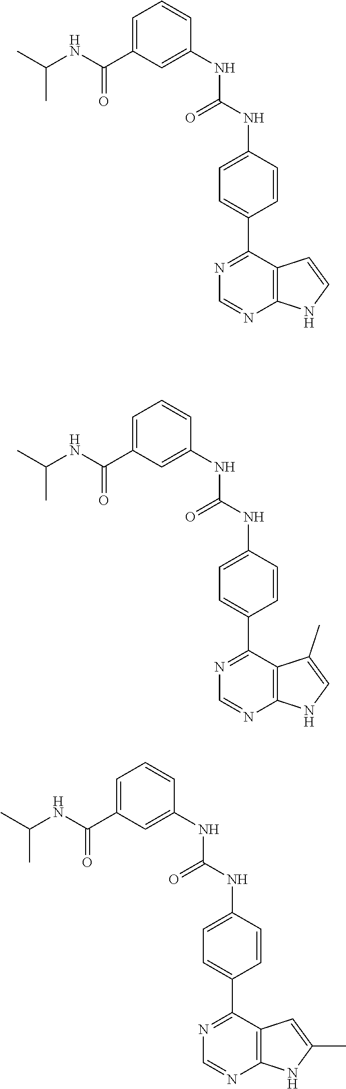 [SMILES: CC(C)NC(=O)c1cccc(NC(=O)Nc2ccc(-c3ncnc4[nH]ccc34)cc2)c1 ; Cc1c[nH]c2ncnc(-c3ccc(NC(=O)Nc4cccc(C(=O)NC(C)C)c4)cc3)c12 ; Cc1cc2c(-c3ccc(NC(=O)Nc4cccc(C(=O)NC(C)C)c4)cc3)ncnc2[nH]1] 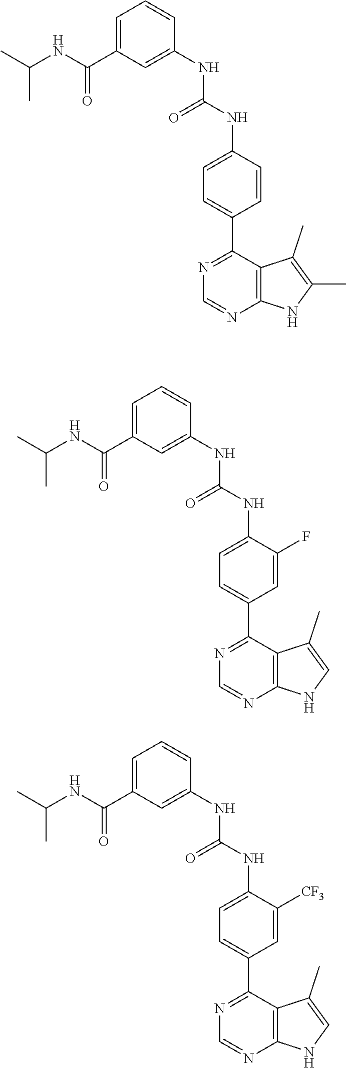 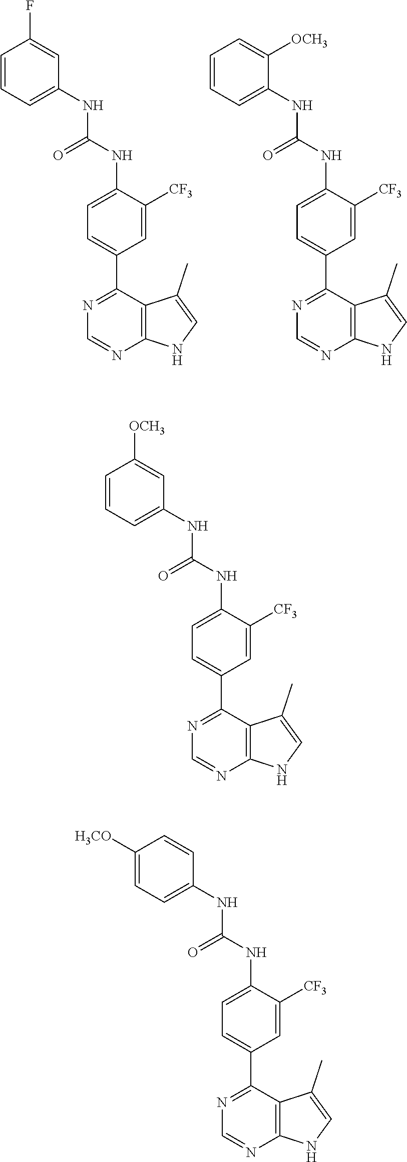 [SMILES: Cc1c[nH]c2ncnc(-c3ccc(NC(=O)Nc4cccc(F)c4)c(C(F)(F)F)c3)c12 ; COc1ccccc1NC(=O)Nc1ccc(-c2ncnc3[nH]cc(C)c23)cc1C(F)(F)F ; COc1cccc(NC(=O)Nc2ccc(-c3ncnc4[nH]cc(C)c34)cc2C(F)(F)F)c1 ; COc1ccc(NC(=O)Nc2ccc(-c3ncnc4[nH]cc(C)c34)cc2C(F)(F)F)cc1] 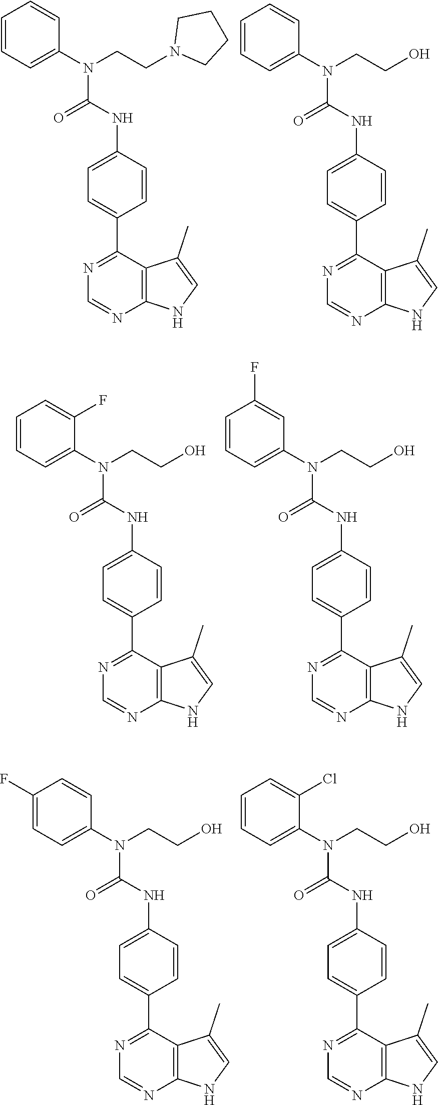 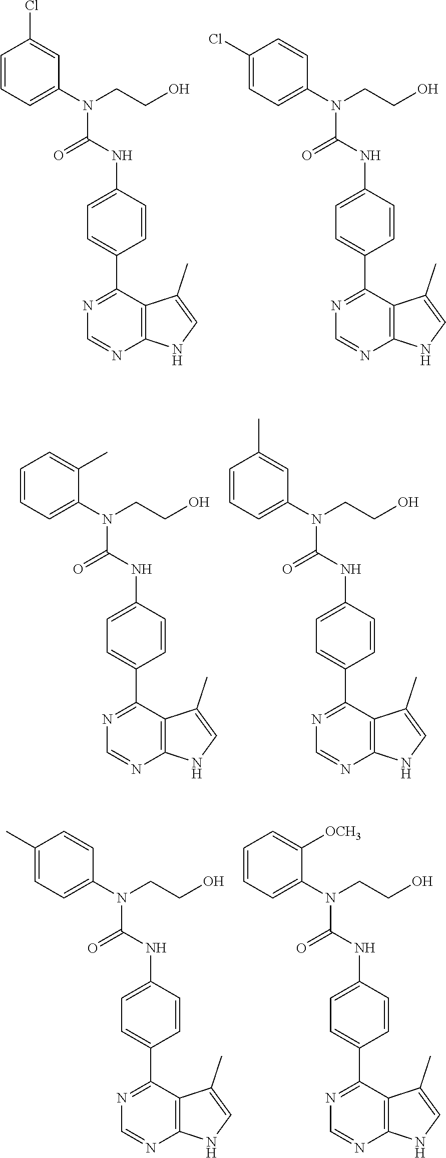 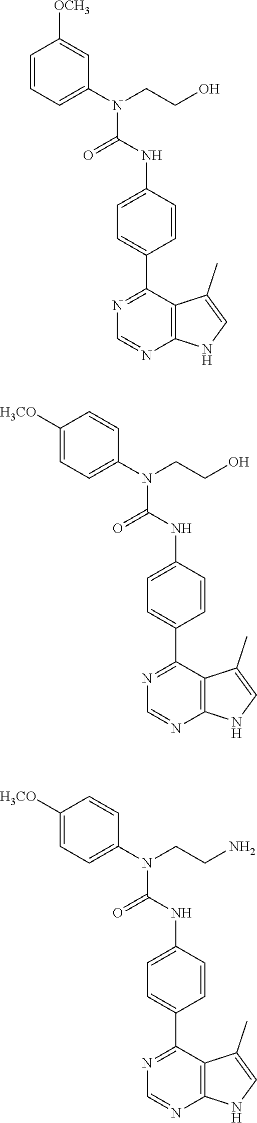 [SMILES: COc1cccc(N(CCO)C(=O)Nc2ccc(-c3ncnc4[nH]cc(C)c34)cc2)c1 ; COc1ccc(N(CCO)C(=O)Nc2ccc(-c3ncnc4[nH]cc(C)c34)cc2)cc1 ; COc1ccc(N(CCN)C(=O)Nc2ccc(-c3ncnc4[nH]cc(C)c34)cc2)cc1] 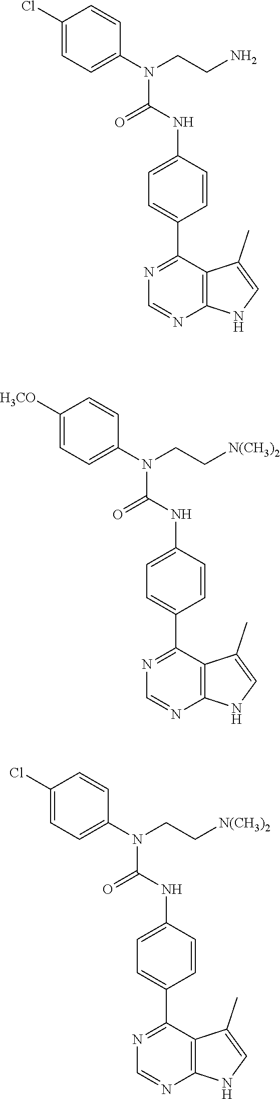 [SMILES: Cc1c[nH]c2ncnc(-c3ccc(NC(=O)N(CCN)c4ccc(Cl)cc4)cc3)c12 ; COc1ccc(N(CCN(C)C)C(=O)Nc2ccc(-c3ncnc4[nH]cc(C)c34)cc2)cc1 ; Cc1c[nH]c2ncnc(-c3ccc(NC(=O)N(CCN(C)C)c4ccc(Cl)cc4)cc3)c12] 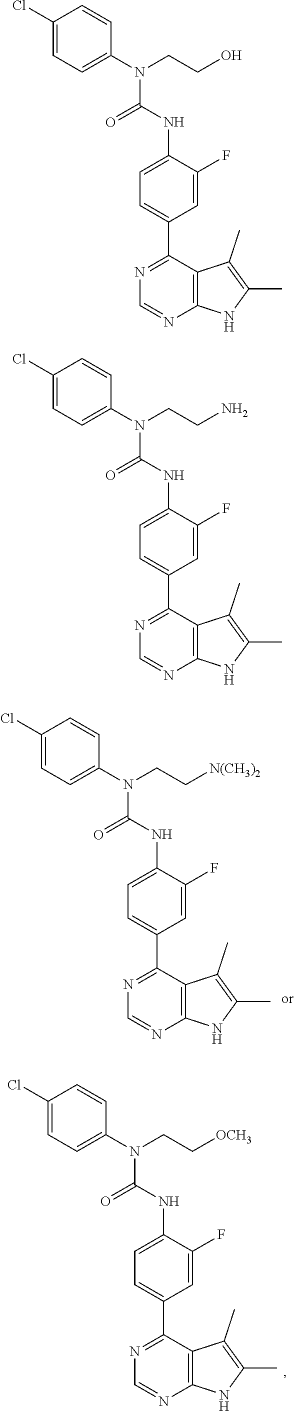 [SMILES: Cc1[nH]c2ncnc(-c3ccc(NC(=O)N(CCO)c4ccc(Cl)cc4)c(F)c3)c2c1C ; Cc1[nH]c2ncnc(-c3ccc(NC(=O)N(CCN)c4ccc(Cl)cc4)c(F)c3)c2c1C ; Cc1[nH]c2ncnc(-c3ccc(NC(=O)N(CCN(C)C)c4ccc(Cl)cc4)c(F)c3)c2c1C ; COCCN(C(=O)Nc1ccc(-c2ncnc3[nH]c(C)c(C)c23)cc1F)c1ccc(Cl)cc1]
or a pharmaceutically acceptable salt thereof.

5. A method of treating a condition in a patient, wherein modulating a LIM kinase is medically indicated, comprising administering to the patient an effective amount of a compound of formula (I) 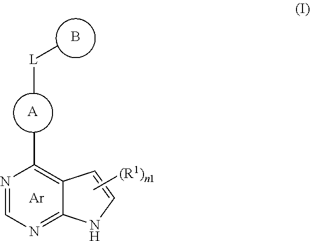
wherein
R¹ is halo, cyano (C₁-C₆)alkoxy, (C₁-C₆)alkyl, (C₂-C₆)alkenyl, or (C₂-C₆)alkynyl, wherein any alkyl, alkenyl, or alkynyl can be unsubstituted or substituted; wherein any one or two methylene groups thereof can be substituted with any of an independently selected NR′, S, O, C(═S), C(═O), OC(═O), OC(═O)O, OC(═O)NR′, C(═O)C(═O), SO₂NR′, S(O), S(O)₂, or C(═O)NR′NR′, wherein each independently selected R′ is H or (C₁-C₆)alkyl or (C₃-C₇)cycloalkyl;
n1=0, 1, or 2;
ring A is a 6-membered saturated ring comprising one or two nitrogen atoms, wherein the nitrogen atoms are disposed at the positions of ring A bonded to linker L, or to ring B when L is a direct bond, and to the ring system Ar; or ring A is a 5- or 6-membered aryl ring, a 5- or 6-membered heteroaryl ring, or a fused 6:5 heteroaryl ring system; ring A is substituted with n2 R² groups, wherein R² is halo, halo(C₁-C₆)alkyl, cyano, (C₁-C₆)alkoxy, (C₁-C₆)alkyl, (C₂-C₆)alkenyl, or (C₂-C₆)alkynyl, wherein any alkyl, alkenyl, or alkynyl can be unsubstituted or substituted; wherein any one or two methylene groups thereof can be substituted with any of an independently selected NR′, S, O, C(═S), C(═O), OC(═O), OC(═O)O, OC(═O)NR′, C(═O)C(═O), SO₂NR′, S(O), S(O)₂, or C(═O)NR′NR′, wherein each independently selected R′ is H or (C₁-C₆)alkyl or (C₃-C₇)cycloalkyl;
n2=0, 1, or 2;
L is a direct bond between ring A and ring B, or L is N(R³)C(═O)N(R³), wherein each R³ is independently H or (C₁-C₆)alkyl, wherein the R³ alkyl can be substituted with hydroxyl, (C₁-C₆)alkoxyl, amino, mono- or di-(C₁-C₆)alkylamino, or a 4- to 7-membered heterocyclyl ring;
ring B is a 5- or 6-membered aryl ring, a 5-membered or a 6-membered heteroaryl ring, or a fused 6:5 heteroaryl ring system; ring B is substituted with n4 R⁴ groups, wherein R⁴ is halo, cyano, (C₁-C₆)alkyl, (C₂-C₆)alkenyl, (C₂-C₆)alkynyl, (C₁-C₆)alkoxy, (C₁-C₆)alkoxycarbonyl, (C₃-C₇)cycloalkoxycarbonyl, R′NHC(═O), or R′₂NC(═O), wherein any alkyl, alkenyl, or alkynyl can be unsubstituted or substituted; wherein any one or two methylene groups thereof can be substituted with any of an independently selected NR′, S, O, C(═S), C(═O), OC(═O), OC(═O)O, OC(═O)NR′, C(═O)C(═O), SO₂NR′, S(O), S(O)₂, or C(═O)NR′NR′, wherein each independently selected R′ is H or (C₁-C₆)alkyl or (C₃-C₇)cycloalkyl;
or a pharmaceutically acceptable salt thereof.

6. The method of claim 5, wherein the compound of formula (I) is a compound of formula (IA) 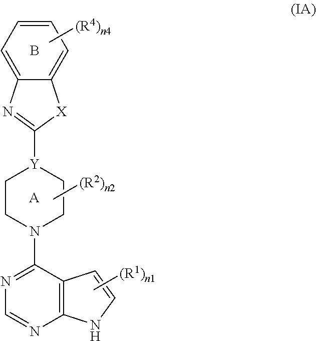
wherein
Y is CR³ or N;
X is NR³, CH₂, CHR¹, O, or S;
wherein R¹, n1, R², n2, R³, R⁴, and n4, are as defined in claim 5.

7. The method of claim 5, wherein the compound of formula (I) is a compound of formula (IB) 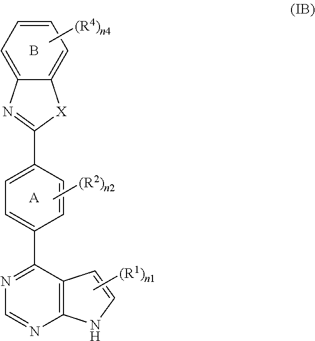
X is NR³, CH₂, CHR¹, O, or S;
wherein R¹, n1, R², n2, R³, R⁴, and n4, are as defined in claim 1.

8. The method of claim 5, wherein the compound of formula (I) is 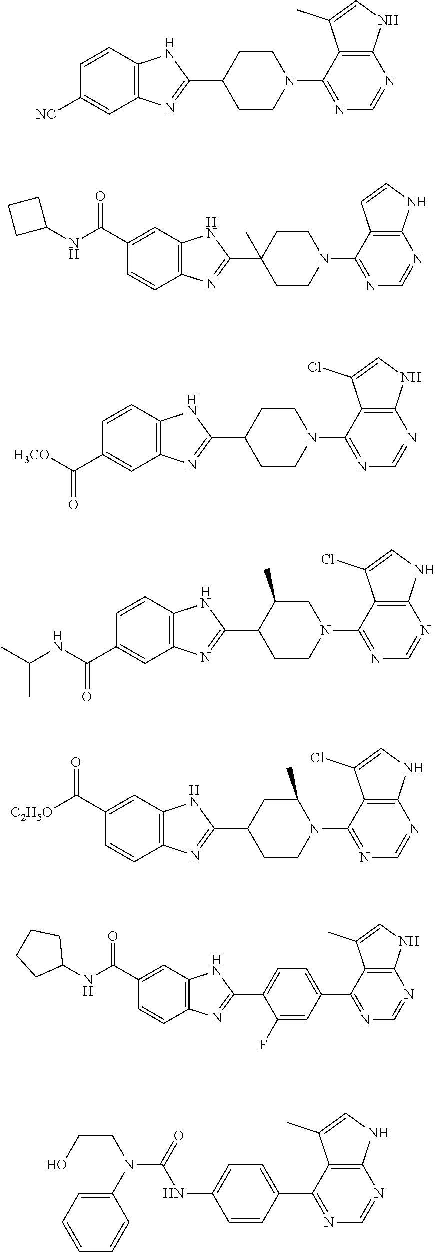 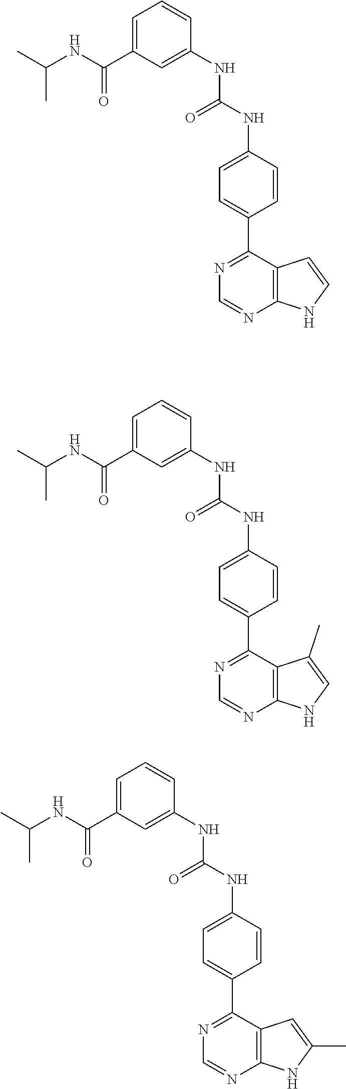 [SMILES: CC(C)NC(=O)c1cccc(NC(=O)Nc2ccc(-c3ncnc4[nH]ccc34)cc2)c1 ; Cc1c[nH]c2ncnc(-c3ccc(NC(=O)Nc4cccc(C(=O)NC(C)C)c4)cc3)c12 ; Cc1cc2c(-c3ccc(NC(=O)Nc4cccc(C(=O)NC(C)C)c4)cc3)ncnc2[nH]1] 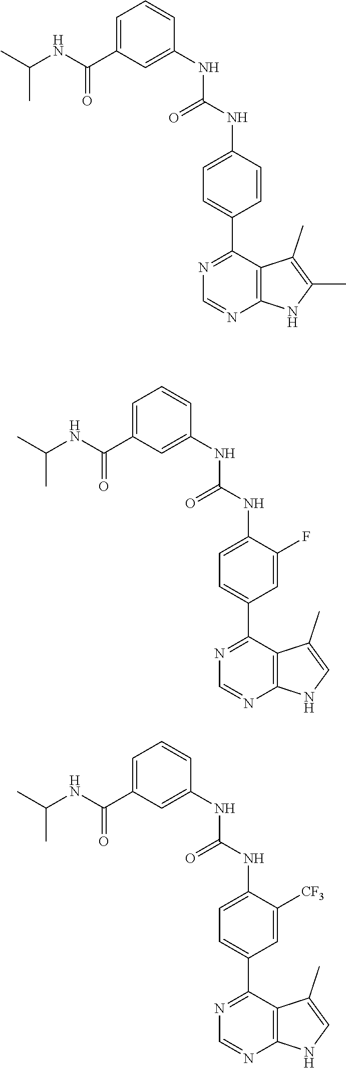 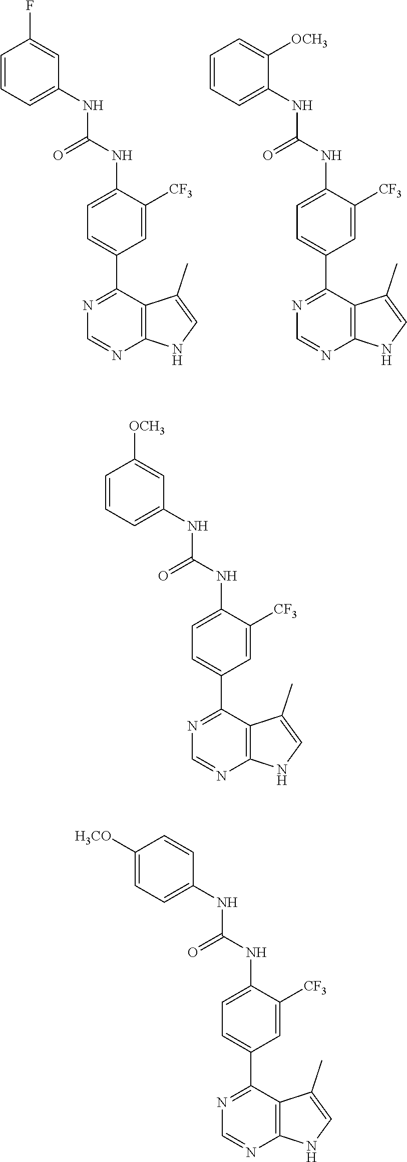 [SMILES: Cc1c[nH]c2ncnc(-c3ccc(NC(=O)Nc4cccc(F)c4)c(C(F)(F)F)c3)c12 ; COc1ccccc1NC(=O)Nc1ccc(-c2ncnc3[nH]cc(C)c23)cc1C(F)(F)F ; COc1cccc(NC(=O)Nc2ccc(-c3ncnc4[nH]cc(C)c34)cc2C(F)(F)F)c1 ; COc1ccc(NC(=O)Nc2ccc(-c3ncnc4[nH]cc(C)c34)cc2C(F)(F)F)cc1] 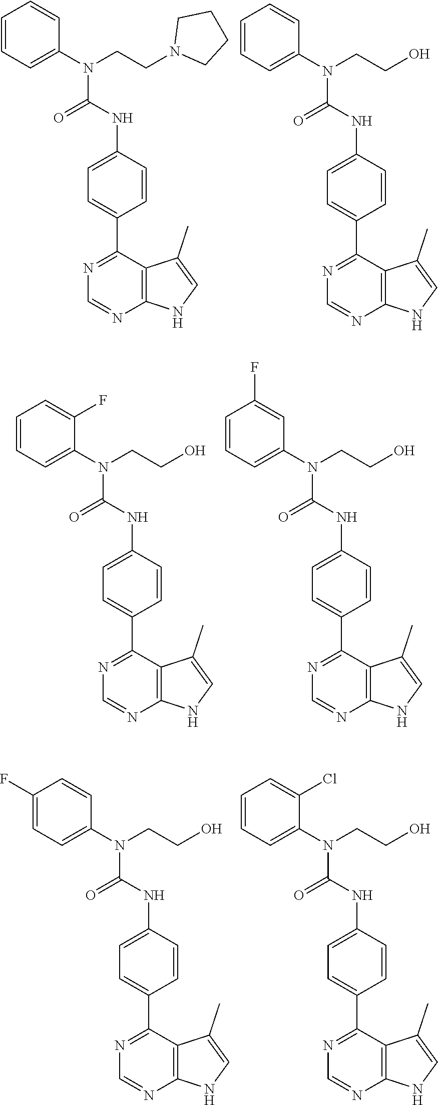 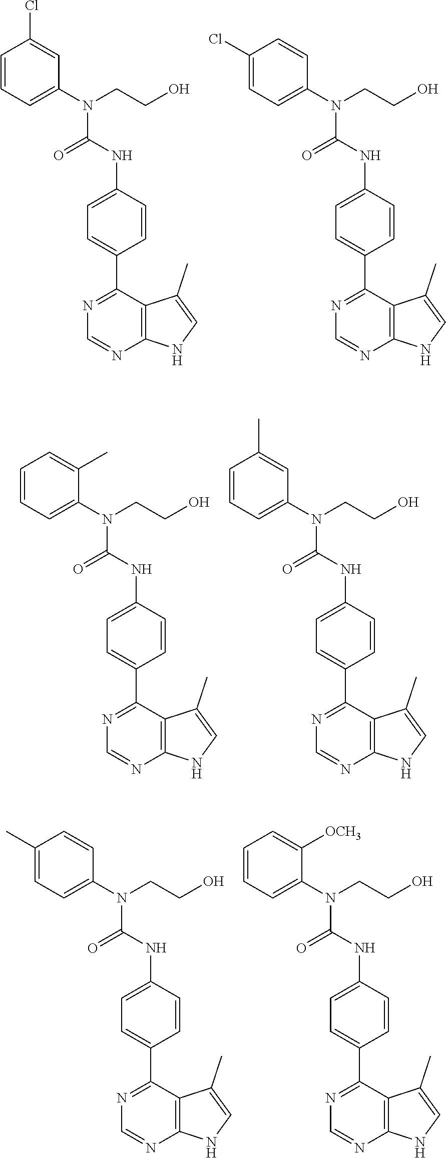 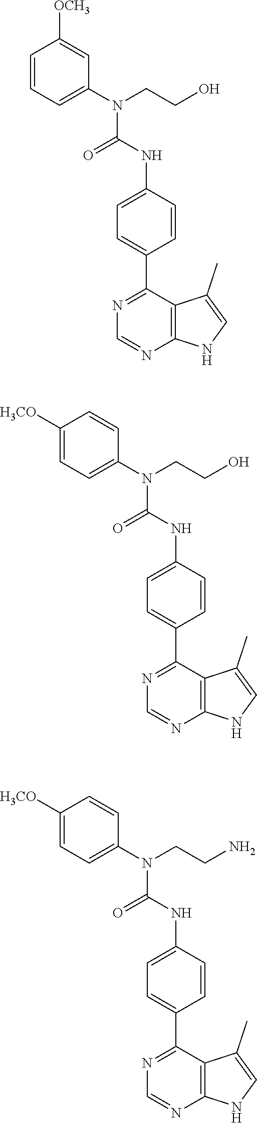 [SMILES: COc1cccc(N(CCO)C(=O)Nc2ccc(-c3ncnc4[nH]cc(C)c34)cc2)c1 ; COc1ccc(N(CCO)C(=O)Nc2ccc(-c3ncnc4[nH]cc(C)c34)cc2)cc1 ; COc1ccc(N(CCN)C(=O)Nc2ccc(-c3ncnc4[nH]cc(C)c34)cc2)cc1] 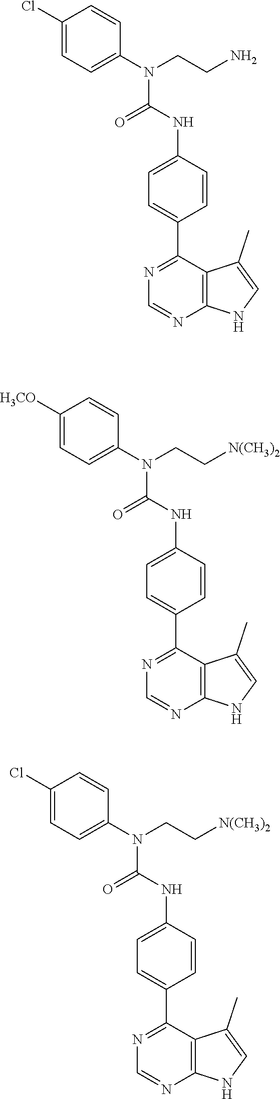 [SMILES: Cc1c[nH]c2ncnc(-c3ccc(NC(=O)N(CCN)c4ccc(Cl)cc4)cc3)c12 ; COc1ccc(N(CCN(C)C)C(=O)Nc2ccc(-c3ncnc4[nH]cc(C)c34)cc2)cc1 ; Cc1c[nH]c2ncnc(-c3ccc(NC(=O)N(CCN(C)C)c4ccc(Cl)cc4)cc3)c12] 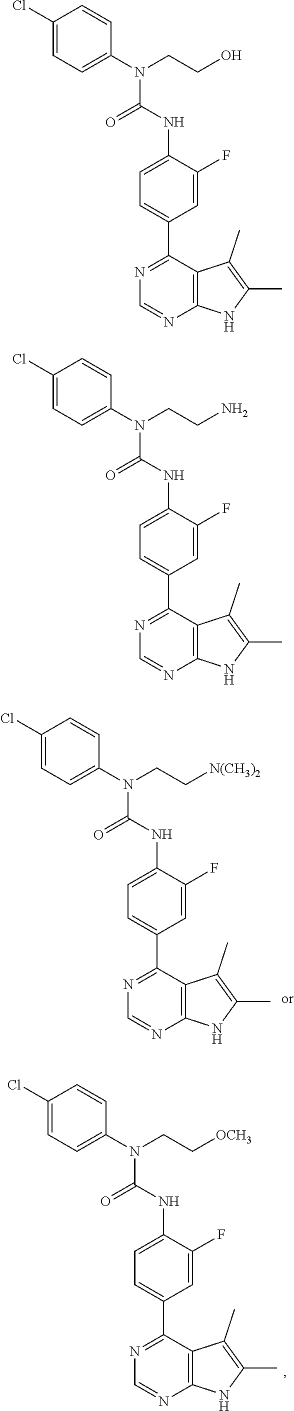 [SMILES: Cc1[nH]c2ncnc(-c3ccc(NC(=O)N(CCO)c4ccc(Cl)cc4)c(F)c3)c2c1C ; Cc1[nH]c2ncnc(-c3ccc(NC(=O)N(CCN)c4ccc(Cl)cc4)c(F)c3)c2c1C ; Cc1[nH]c2ncnc(-c3ccc(NC(=O)N(CCN(C)C)c4ccc(Cl)cc4)c(F)c3)c2c1C ; COCCN(C(=O)Nc1ccc(-c2ncnc3[nH]c(C)c(C)c23)cc1F)c1ccc(Cl)cc1]
or a pharmaceutically acceptable salt thereof.

9. The method of claim 5, wherein the condition is a viral infection, a metastatic condition, a bacterial infection, Alzheimer's disease, glaucoma, psoriasis, a sexually transmitted disease, or a drug addiction.

10. The method of claim 9, wherein the viral infection is a human immunodeficiency viral (HIV) infection, an Ebola viral (EBOV) infection, a Rift Valley Fever viral (RVFV) infection, a Venezuelan equine encephalitis viral (VEEV) infection, or a Herpes Simplex 1 viral (HSV-1) infection; and wherein the bacterial infection is a Chlamydia infection.

11. A compound of formula (I) 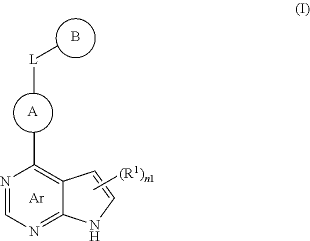
wherein
R¹ is halo, cyano (C₁-C₆)alkoxy, (C₁-C₆)alkyl, (C₂-C₆)alkenyl, or (C₂-C₆)alkynyl, wherein any alkyl, alkenyl, or alkynyl can be unsubstituted or substituted; wherein any one or two methylene groups thereof can be substituted with any of an independently selected NR′, S, O, C(═S), C(═O), OC(═O), OC(═O)O, OC(═O)NR′, C(═O)C(═O), SO₂NR′, S(O), S(O)₂, or C(═O)NR′NR′, wherein each independently selected R′ is H or (C₁-C₆)alkyl or (C₃-C₇)cycloalkyl;
n1=0, 1, or 2;
ring A is a 6-membered saturated ring comprising one or two nitrogen atoms, wherein the nitrogen atoms are disposed at the positions of ring A bonded to linker L, or to ring B when L is a direct bond, and to the ring system Ar; or ring A is a 5- or 6-membered aryl ring, a 5- or 6-membered heteroaryl ring, or a fused 6:5 heteroaryl ring system; ring A is substituted with n2
R² groups, wherein R² is halo, halo(C₁-C₆)alkyl, cyano, (C₁-C₆)alkoxy, (C₁-C₆)alkyl, (C₂-C₆)alkenyl, or (C₂-C₆)alkynyl, wherein any alkyl, alkenyl, or alkynyl can be unsubstituted or substituted; wherein any one or two methylene groups thereof can be substituted with any of an independently selected NR′, S, O, C(═S), C(═O), OC(═O), OC(═O)O, OC(═O)NR′, C(═O)C(═O), SO₂NR′, S(O), S(O)₂, or C(═O)NR′NR′, wherein each independently selected R′ is H or (C₁-C₆)alkyl or (C₃-C₇)cycloalkyl;
n2=0, 1, or 2;
L is a direct bond between ring A and ring B, or L is N(R³)C(═O)N(R³), wherein each R³ is independently H or (C₁-C₆)alkyl, wherein the R³ alkyl can be substituted with hydroxyl, (C₁-C₆)alkoxyl, amino, mono- or di-(C₁-C₆)alkylamino, or a 4- to 7-membered heterocyclyl ring;
ring B is a 5- or 6-membered aryl ring, a 5-membered or a 6-membered heteroaryl ring, or a fused 6:5 heteroaryl ring system; ring B is substituted with n4 R⁴ groups, wherein R⁴ is halo, cyano, (C₁-C₆)alkyl, (C₂-C₆)alkenyl, (C₂-C₆)alkynyl, (C₁-C₆)alkoxy, (C₁-C₆)alkoxycarbonyl, (C₃-C₇)cycloalkoxycarbonyl, R′NHC(═O), or R′₂NC(═O), wherein any alkyl, alkenyl, or alkynyl can be unsubstituted or substituted; wherein any one or two methylene groups thereof can be substituted with any of an independently selected NR′, S, O, C(═S), C(═O), OC(═O), OC(═O)O, OC(═O)NR′, C(═O)C(═O), SO₂NR′, S(O), S(O)₂, or C(═O)NR′NR′, wherein each independently selected R′ is H or (C₁-C₆)alkyl or (C₃-C₇)cycloalkyl;
or a pharmaceutically acceptable salt thereof.

12. The compound of claim 11, wherein the compound of formula (I) is a compound of formula (IA) 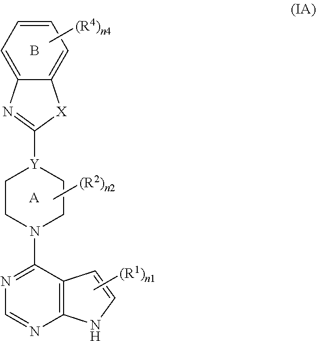
wherein
Y is CR³ or N;
X is NR³, CH₂, CHR¹, O, or S;
wherein R¹, n1, R², n2, R³, R⁴, and n4, are as defined in claim 1.

13. The compound of claim 11, wherein the compound of formula (I) is a compound of formula (IB) 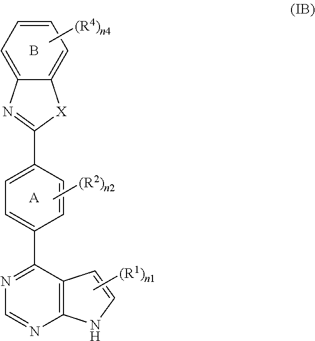
X is NR³, CH₂, CHR¹, O, or S;
wherein R¹, n1, R², n2, R³, R⁴, and n4, are as defined in claim 1.

14. The compound of claim 11, wherein the compound of formula (I) is 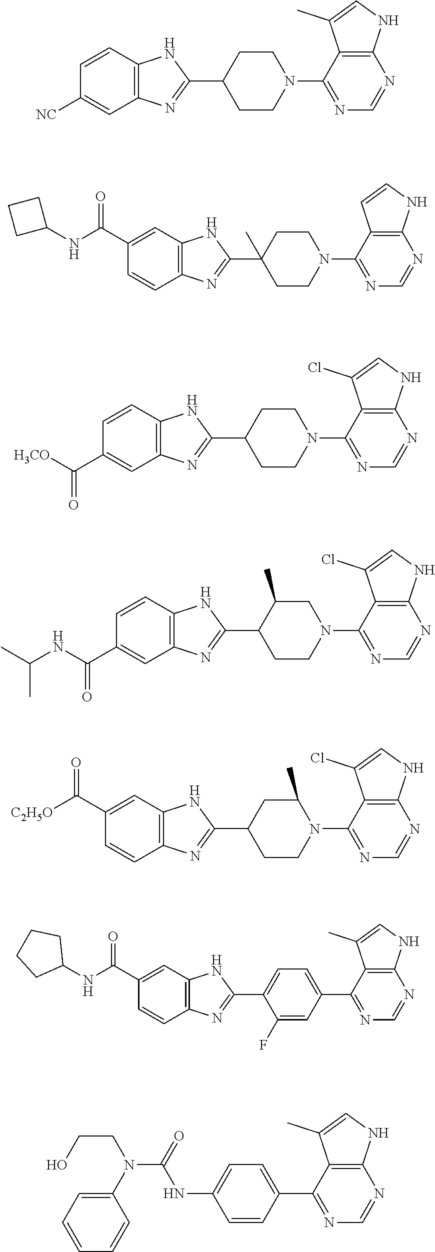 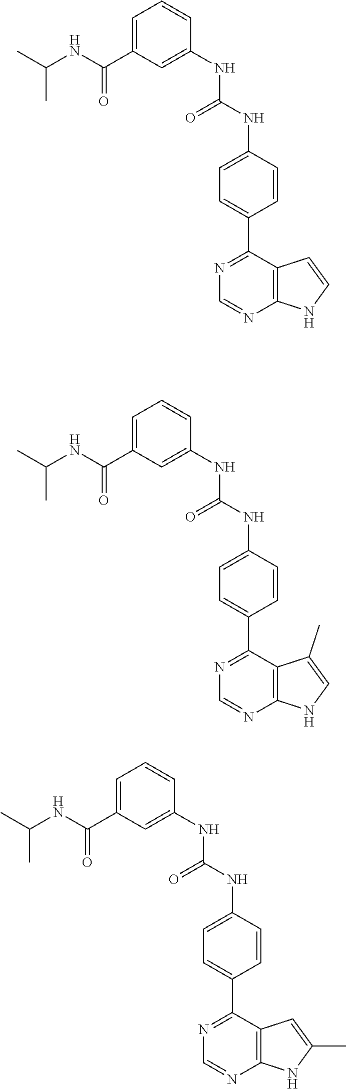 [SMILES: CC(C)NC(=O)c1cccc(NC(=O)Nc2ccc(-c3ncnc4[nH]ccc34)cc2)c1 ; Cc1c[nH]c2ncnc(-c3ccc(NC(=O)Nc4cccc(C(=O)NC(C)C)c4)cc3)c12 ; Cc1cc2c(-c3ccc(NC(=O)Nc4cccc(C(=O)NC(C)C)c4)cc3)ncnc2[nH]1] 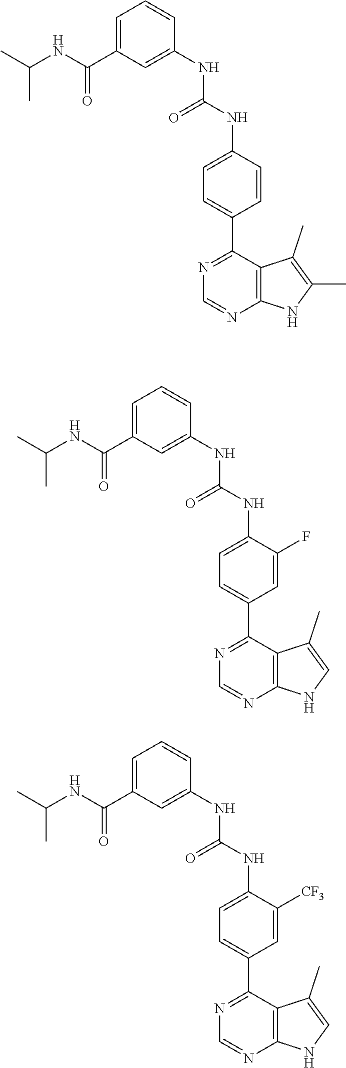 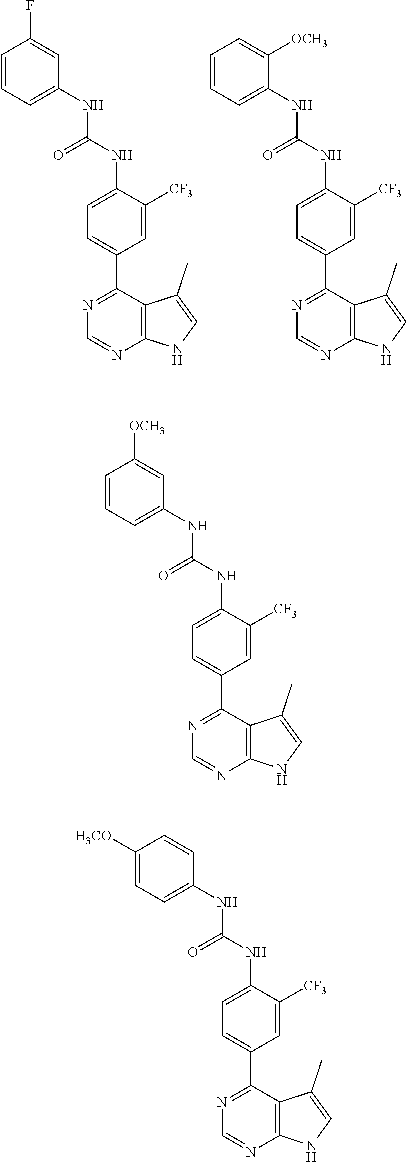 [SMILES: Cc1c[nH]c2ncnc(-c3ccc(NC(=O)Nc4cccc(F)c4)c(C(F)(F)F)c3)c12 ; COc1ccccc1NC(=O)Nc1ccc(-c2ncnc3[nH]cc(C)c23)cc1C(F)(F)F ; COc1cccc(NC(=O)Nc2ccc(-c3ncnc4[nH]cc(C)c34)cc2C(F)(F)F)c1 ; COc1ccc(NC(=O)Nc2ccc(-c3ncnc4[nH]cc(C)c34)cc2C(F)(F)F)cc1] 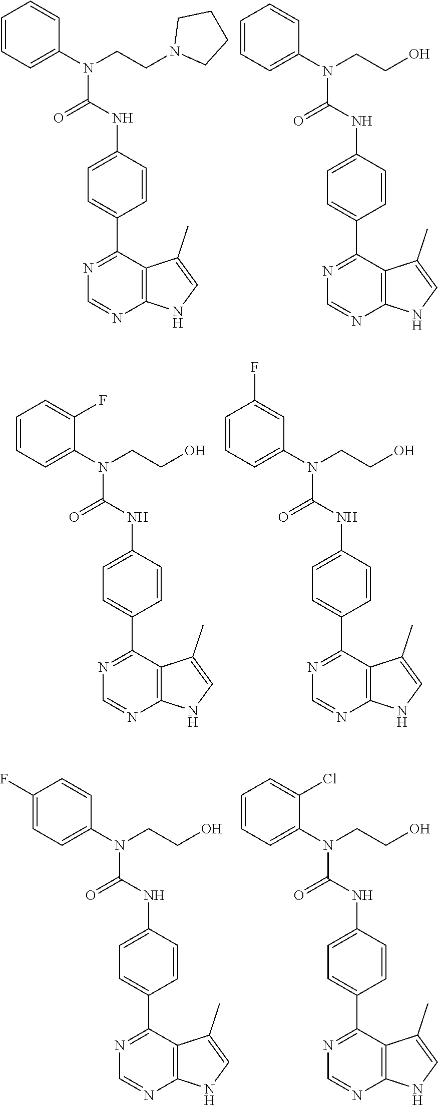 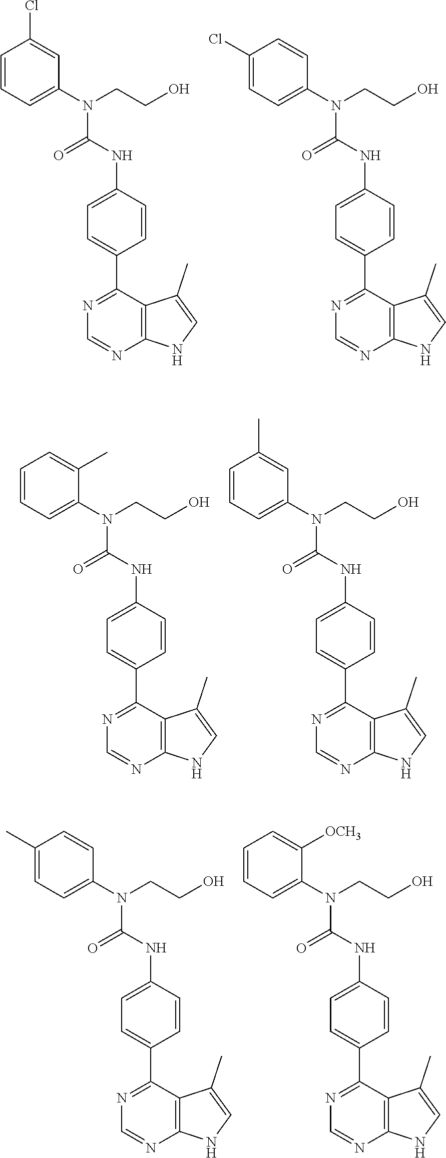 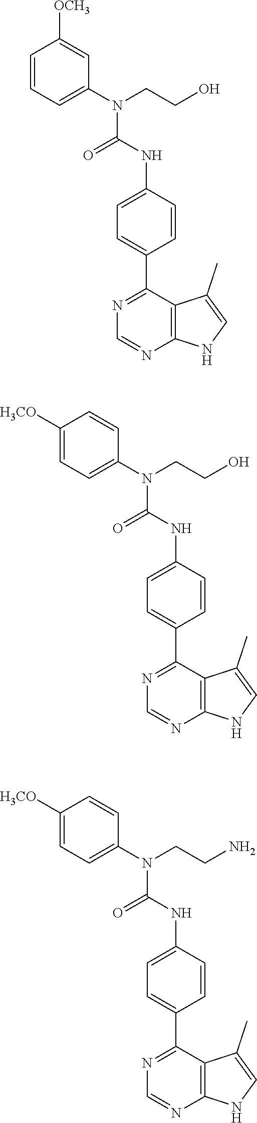 [SMILES: COc1cccc(N(CCO)C(=O)Nc2ccc(-c3ncnc4[nH]cc(C)c34)cc2)c1 ; COc1ccc(N(CCO)C(=O)Nc2ccc(-c3ncnc4[nH]cc(C)c34)cc2)cc1 ; COc1ccc(N(CCN)C(=O)Nc2ccc(-c3ncnc4[nH]cc(C)c34)cc2)cc1] 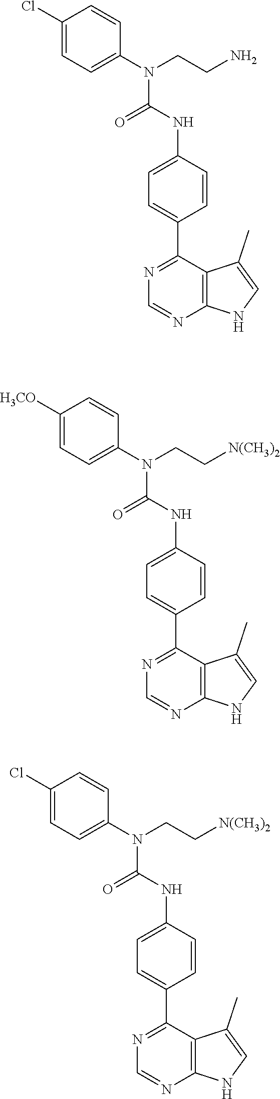 [SMILES: Cc1c[nH]c2ncnc(-c3ccc(NC(=O)N(CCN)c4ccc(Cl)cc4)cc3)c12 ; COc1ccc(N(CCN(C)C)C(=O)Nc2ccc(-c3ncnc4[nH]cc(C)c34)cc2)cc1 ; Cc1c[nH]c2ncnc(-c3ccc(NC(=O)N(CCN(C)C)c4ccc(Cl)cc4)cc3)c12] 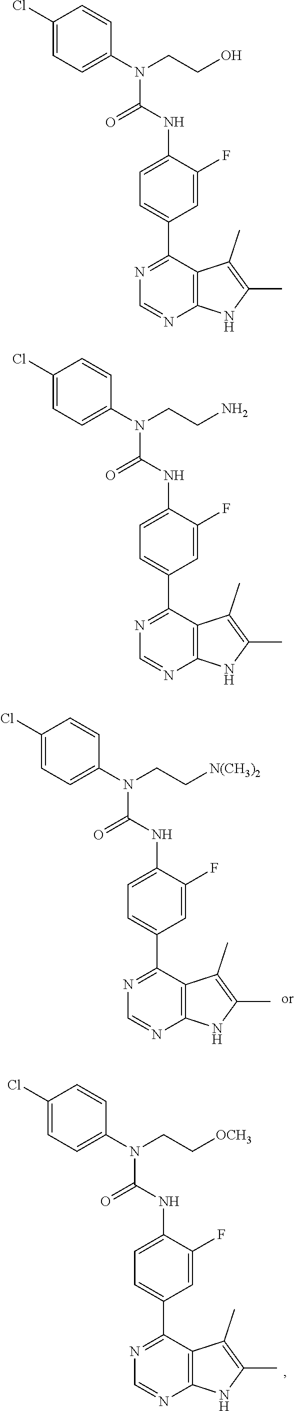 [SMILES: Cc1[nH]c2ncnc(-c3ccc(NC(=O)N(CCO)c4ccc(Cl)cc4)c(F)c3)c2c1C ; Cc1[nH]c2ncnc(-c3ccc(NC(=O)N(CCN)c4ccc(Cl)cc4)c(F)c3)c2c1C ; Cc1[nH]c2ncnc(-c3ccc(NC(=O)N(CCN(C)C)c4ccc(Cl)cc4)c(F)c3)c2c1C ; COCCN(C(=O)Nc1ccc(-c2ncnc3[nH]c(C)c(C)c23)cc1F)c1ccc(Cl)cc1]
or a pharmaceutically acceptable salt thereof.

15. The method of claim 6, wherein the condition is a viral infection, a metastatic condition, a bacterial infection, Alzheimer's disease, glaucoma, psoriasis, a sexually transmitted disease, or a drug addiction.

16. The method of claim 15, wherein the viral infection is a human immunodeficiency viral (HIV) infection, an Ebola viral (EBOV) infection, a Rift Valley Fever viral (RVFV) infection, a Venezuelan equine encephalitis viral (VEEV) infection, or a Herpes Simplex 1 viral (HSV-1) infection; and wherein the bacterial infection is a Chlamydia infection.

17. The method of claim 7, wherein the condition is a viral infection, a metastatic condition, a bacterial infection, Alzheimer's disease, glaucoma, psoriasis, a sexually transmitted disease, or a drug addiction.

18. The method of claim 17, wherein the viral infection is a human immunodeficiency viral (HIV) infection, an Ebola viral (EBOV) infection, a Rift Valley Fever viral (RVFV) infection, a Venezuelan equine encephalitis viral (VEEV) infection, or a Herpes Simplex 1 viral (HSV-1) infection; and wherein the bacterial infection is a Chlamydia infection.

19. The method of claim 8, wherein the condition is a viral infection, a metastatic condition, a bacterial infection, Alzheimer's disease, glaucoma, psoriasis, a sexually transmitted disease, or a drug addiction.

20. The method of claim 19, wherein the viral infection is a human immunodeficiency viral (HIV) infection, an Ebola viral (EBOV) infection, a Rift Valley Fever viral (RVFV) infection, a Venezuelan equine encephalitis viral (VEEV) infection, or a Herpes Simplex 1 viral (HSV-1) infection; and wherein the bacterial infection is a Chlamydia infection.
